SEQUENCE LISTING (106 sequences)

SEQ ID NO: 1 - protein, 201 aa, synthetic construct
  source 1..201: note = TCR D046C1 alpha chain full-length wild-type sequence
KNEVEQSPQNLTAQEGEFITINCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 2 - protein, 110 aa, synthetic construct
  source 1..110: note = TCR D046C1 alpha chain variable region wild-type sequence
KNEVEQSPQNLTAQEGEFITINCSYSVGISALHWLQQHPGGGIVSLFMLSSGKKKHGRLIATINIQEKHSSLHITASHPRDSAVYICAADPSDSWGKLQFGAGTQVVVTP

SEQ ID NO: 3 - protein, 91 aa, synthetic construct
  source 1..91: note = TCR D046C1 alpha chain constant region wild-type sequence
DIQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 4 - protein, 247 aa, synthetic construct
  source 1..247: note = TCR D046C1 beta chain full-length wild-type sequence
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSESRILTYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 5 - protein, 116 aa, synthetic construct
  source 1..116: note = TCR D046C1 beta chain variable region wild-type sequence
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSESRILTYNEQFFGPGTRLTVL

SEQ ID NO: 6 - protein, 131 aa, synthetic construct
  source 1..131: note = TCR D046C1 beta chain constant region wild-type sequence
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADC

SEQ ID NO: 7 - protein, 9 aa, synthetic construct
  source 1..9: note = HLA-A*02:01 restricted CMV pp65 epitope
NLVPMVATV

SEQ ID NO: 8 - protein, 365 aa, synthetic construct [sequence omitted]

SEQ ID NO: 9 - protein, 278 aa, synthetic construct [sequence omitted]

SEQ ID NO: 10 - protein, 119 aa, synthetic construct
  source 1..119: note = full length human beta-2-microglobulin
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 11 - protein, 201 aa, synthetic construct
  source 1..201: note = TCR D046C1 alpha chain Mutant 1
KNEVEQSPQNLTAQEGEFITIQCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTQVSQSKDSDVYITDKCVLDMRSMDFKSNSAVAWSQKSDFACANAFNNSIIPEDTFFPSP

SEQ ID NO: 12 - protein, 201 aa, synthetic construct
  source 1..201: note = TCR D046C1 alpha chain Mutant 2
KNEVEQSPQNLTAQEGEFITIQCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKCVLDMRSMDFKSNSAVAWSQKSDFACANAFQNSIIPEDTFFPSP

SEQ ID NO: 13 - protein, 247 aa, synthetic construct
  source 1..247: note = TCR D046C1 beta chain Mutant 1
EPEVTQTPSHQVTQMGQEVILRCVPISNHLYFYWYRQILGQKVEFLVSFYNNEISEKSEIFDDQFSVERPDGSQFTLKIRSTKLEDSAMYFCASSESRILTYNEQFFGPGTRLTVLEDLNKVFPPEVAVFEPSEAEISHTQKATLVCLATGFFPDHVELSWWVNGKEVHSGVCTDPQPLKEQPALQDSRYCLSSRLRVSATFWQNPRNHFRCQVQFYGLSENDEWTQDRAKPVTQIVSAEAWGRADC

SEQ ID NO: 14 - protein, 247 aa, synthetic construct
  source 1..247: note = TCR D046C1 beta chain Mutant 2
EPEVTQTPSHQVTQMGQEVILRCVPISNHLYFYWYRQILGQKVEFLVSFYNNEISEKSEIFDDQFSVERPDGSQFTLKIRSTKLEDSAMYFCASSESRILTYNEQFFGPGTRLTVLEDLNKVFPPEVAVFEPSEAEISHTQKATLVCLATGFFPDHVELSWWVNGKEVHSGVCTDPQPLKEQPALQDSRYALSSRLRVSATFWQNPRNHFRCQVQFYGLSENDEWTQDRAKPVTQIVSAEAWGRADC

SEQ ID NO: 15 - protein, 137 aa, synthetic construct
  source 1..137: note = Human Immunoglobulin J chain
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 16 - protein, 240 aa, synthetic construct
  source 1..240: note = Anti-CD3 scFv sequence
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIK

SEQ ID NO: 17 - protein, 411 aa, synthetic construct
  source 1..411: note = Human Ig J chain with anti-CD3 at C-terminus + tags
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSDYKDDDDKHHHHHH

SEQ ID NO: 18 - protein, 411 aa, synthetic construct
  source 1..411: note = Human Ig J chain with anti-CD3 at N-terminus + tags
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSDYKDDDDKHHHHHH

SEQ ID NO: 19 - protein, 15 aa, synthetic construct
  source 1..15: note = linker
GGGGSGGGGSGGGGS

SEQ ID NO: 20 - protein, 22 aa, synthetic construct
  source 1..22: note = linker
GSAPKPAPKPAPAPKPAPKPAP

SEQ ID NO: 21 - protein, 40 aa, synthetic construct
  source 1..40: note = linker
GGGGSGGGGSGGGGSGGGGSAPKPAPKPAPAPKPAPKPAP

SEQ ID NO: 22 - protein, 5 aa, synthetic construct
  source 1..5: note = linker
  REPEAT 1..5: note = n, wherein n=1-6
GGGGS

SEQ ID NO: 23 - protein, 13 aa, synthetic construct
  source 1..13: note = linker
SSSSGSSSSGSAA

SEQ ID NO: 24 - protein, 5 aa, synthetic construct
  source 1..5: note = linker
GGGGG

SEQ ID NO: 25 - protein, 6 aa, synthetic construct
  source 1..6: note = linker
  REPEAT 2..6: note = n, wherein n=1-10
SGGGGS

SEQ ID NO: 26 - protein, 4 aa, synthetic construct
  source 1..4: note = linker
  REPEAT 1..4: note = n, wherein n=1-5
GGSG

SEQ ID NO: 27 - protein, 4 aa, synthetic construct
  source 1..4: note = linker
GSAT

SEQ ID NO: 28 - protein, 6 aa, synthetic construct
  source 1..6: note = linker
  REPEAT 1..6: note = n, wherein n=1-5
GGSGGS

SEQ ID NO: 29 - protein, 20 aa, synthetic construct
  source 1..20: note = (G4S)4 linker
GGGGSGGGGSGGGGSGGGGS

SEQ ID NO: 30 - protein, 14 aa, synthetic construct
  source 1..14: note = (GS)2AG2SGSG3S linker
GSGSAGGSGSGGGS

SEQ ID NO: 31 - protein, 8 aa, synthetic construct
  source 1..8: note = FLAG tag
DYKDDDDK

SEQ ID NO: 32 - protein, 6 aa, synthetic construct
  source 1..6: note = His6 Tag
HHHHHH

SEQ ID NO: 33 - protein, 14 aa, synthetic construct
  source 1..14: note = V5 Tag
GKPIPNPLLGLDST

SEQ ID NO: 34 - protein, 8 aa, synthetic construct
  source 1..8: note = strep-tag
WSHPQFEK

SEQ ID NO: 35 - protein, 12 aa, synthetic construct
  source 1..12: note = protein c tag
EDQVDPRLIDGK

SEQ ID NO: 36 - protein, 10 aa, synthetic construct
  source 1..10: note = Myc tag
EQKLISEEDL

SEQ ID NO: 37 - protein, 28 aa, synthetic construct
  source 1..28: note = avitag-Myc (biotin-mediated)
GLNDIFEAQKIEWHEGSGEQKLISEEDL

SEQ ID NO: 38 - protein, 34 aa, synthetic construct
  source 1..34: note = avitag-Myc-His (biotin-mediated)
GLNDIFEAQKIEWHEGSGEQKLISEEDLHHHHHH

SEQ ID NO: 39 - protein, 40 aa, synthetic construct
  source 1..40: note = Avitag sequence with His6 tag and Flag tag
GSGSAGGGLNDIFEAQKIEWHEGSTGHHHHHHDYKDDDDK

SEQ ID NO: 40 - protein, 159 aa, synthetic construct
  source 1..159: note = human full-length J chain
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 41 - protein, 18 aa, synthetic construct
  source 1..18: note = human IgM tailpiece
PTLYNVSLVMSDTAGTCY

SEQ ID NO: 42 - protein, 18 aa, synthetic construct
  source 1..18: note = human IgA tailpiece
PTHVNVSVVMAEVDGTCY

SEQ ID NO: 43 - protein, 8 aa, synthetic construct
  SITE 2: note = misc_feature - Xaa can be any naturally occurring amino acid
  SITE 4: note = misc_feature - Xaa can be any naturally occurring amino acid
  source 1..8: note = 2A core sequence motif
DXEXNPGP

SEQ ID NO: 44 - protein, 18 aa, synthetic construct
  source 1..18: note = T2A sequence
EGRGSLLTCGDVEENPGP

SEQ ID NO: 45 - protein, 19 aa, synthetic construct
  source 1..19: note = P2A sequence
ATNFSLLKQAGDVEENPGP

SEQ ID NO: 46 - protein, 20 aa, synthetic construct
  source 1..20: note = E2A sequence
QCTNYALLKLAGDVESNPGP

SEQ ID NO: 47 - protein, 22 aa, synthetic construct
  source 1..22: note = F2A sequence
VKQTLNFDLLKLAGDVESNPGP

SEQ ID NO: 48 - protein, 20 aa, synthetic construct
  source 1..20: note = Ig Kappa chain V-III region CLL signal peptide
MEAPAQLLFLLLLWLPDTTG

SEQ ID NO: 49 - protein, 23 aa, synthetic construct
  source 1..23: note = signal peptide of human CD4 signal peptide
MNRGVPFRHLLLVLQLALLPAAT

SEQ ID NO: 50 - protein, 20 aa, synthetic construct
  source 1..20: note = mouse Ig kappa chain V-III region signal peptide
METDTLLLWVLLLWVPGSTG

SEQ ID NO: 51 - protein, 21 aa, synthetic construct
  source 1..21: note = mouse H-2Kb signal peptide
MVPCTLLLLLAAALAPTQTRA

SEQ ID NO: 52 - protein, 18 aa, synthetic construct
  source 1..18: note = human serum albumin signal peptide
MKWVTFISLLFLFSSAYS

SEQ ID NO: 53 - protein, 20 aa, synthetic construct
  source 1..20: note = human IL-2 signal peptide
MYRMQLLSCIALSLALVTNS

SEQ ID NO: 54 - protein, 24 aa, synthetic construct
  source 1..24: note = human HLA-A*02:01 signal peptide
MAVMAPRTLLLLLSGALALTQTWA

SEQ ID NO: 55 - protein, 20 aa, synthetic construct
  source 1..20: note = Human b2m signal peptide
MSRSVALAVLALLSLSGLEA

SEQ ID NO: 56 - protein, 6 aa, synthetic construct
  source 1..6: note = sortase recognition site
LPETGG

SEQ ID NO: 57 - protein, 561 aa, synthetic construct
  source 1..561: note = TCR D046C1a wt, connecting peptide linker, Cu2-4/amino acid sequence
KNEVEQSPQNLTAQEGEFITINCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 58 - DNA, 1683 bp, synthetic construct
  source 1..1683: mol_type = other DNA; note = TCR D046C1a wt, connecting peptide linker, Cu2-4/nucleotide sequence
aagaatgaagtggagcaatcacctcaaaacctaaccgctcaggagggtgaattcatcaccatcaactgtagctactccgtcggcatctccgcactgcattggctccagcagcatcctggaggtggcatcgtcagcctcttcatgctgtcaagcggtaagaaaaagcatgggaggctgatcgccaccatcaatattcaggaaaaacacagcagtttgcacattacagcttcccacccccgtgactctgccgtgtacatctgcgctgctgacccttctgacagttggggcaaattgcagttcggcgctggcacccaggtcgttgtgaccccggacattcaaaaccccgatccggctgtctaccaactacgagactctaagagctctgacaaatccgtgtgcctttttaccgacttcgatagccaaacaaacgtgtctcagagcaaggacagcgacgtttatatcacagacaagactgtattggacatgcgctccatggacttcaagtccaatagcgctgttgcttggtccaataaaagtgatttcgcttgtgccaacgcttttaacaactctatcattcctgaggacacctttttcccttctcctgaaagttcttgtgatgttaagctggttgaaaagtccatcgcagaactgccacctaaagtcagcgtctttgtgcctccccgtgatggcttttttggcaacccccggaagagtaaactaatatgccaggccacaggatttagtccgcggcagattcaggtgtcttggctccgggagggaaaacaggtcggaagtggagtgaccacagaccaagtgcaggccgaggctaaggagagtggtcctacaacttacaaggttacaagtaccttgacaattaaagagtccgattggctgggacagagtatgtttacatgtagagtggaccaccgtggtctcactttccagcagaatgctagttcaatgtgcgttccggatcaggataccgcaattagagtgttcgccatccctccctcttttgcttccatcttcctgaccaagagcaccaagcttacatgcttggttaccgatcttacaacctatgattctgtcactatctcctggacacgccagaacggggaggccgtgaagacacatactaatatcagcgagagccatcccaacgctacattctcagcagtgggcgaggcctctatctgcgaagacgactggaattcaggggaacggttcacgtgtactgtcacccatacagatctgccttcccctcttaagcagaccatttcacgaccgaaaggggtggctcttcaccggcccgacgtgtacctactcccacctgcccgtgaacaactgaatctgcgcgagagcgctaccattacctgcctggtaaccggatttagtcctgcagacgtctttgttcagtggatgcaacgcggccagcccctctcccctgagaagtatgtgactagtgcccctatgcccgaaccccaggctcctggcagatattttgcccatagcatcctgacagtatcagaagaggagtggaacactggtgaaacctatacttgcgtggtggcacacgaggctttgccgaaccgcgtcacagaacgaaccgttgacaaaagcacaggtaagcccaccctgtacaatgtcagcctcgtaatgtctgatacagcaggtacctgttat

SEQ ID NO: 59 - protein, 568 aa, synthetic construct
  source 1..568: note = TCR D046C1alpha wt, GS linker, Cu2-4/amino acid sequence
KNEVEQSPQNLTAQEGEFITINCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGGGGSGGGGSGGGGSIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 60 - DNA, 1704 bp, synthetic construct
  source 1..1704: mol_type = other DNA; note = TCR D046C1alpha wt, GS linker, Cu2-4/nucleotide sequence
aagaatgaagtggagcaatcacctcaaaacctaaccgctcaggagggtgaattcatcaccatcaactgtagctactccgtcggcatctccgcactgcattggctccagcagcatcctggaggtggcatcgtcagcctcttcatgctgtcaagcggtaagaaaaagcatgggaggctgatcgccaccatcaatattcaggaaaaacacagcagtttgcacattacagcttcccacccccgtgactctgccgtgtacatctgcgctgctgacccttctgacagttggggcaaattgcagttcggcgctggcacccaggtcgttgtgaccccggacattcaaaaccccgatccggctgtctaccaactacgagactctaagagctctgacaaatccgtgtgcctttttaccgacttcgatagccaaacaaacgtgtctcagagcaaggacagcgacgtttatatcacagacaagactgtattggacatgcgctccatggacttcaagtccaatagcgctgttgcttggtccaataaaagtgatttcgcttgtgccaacgcttttaacaactctatcattcctgaggacacctttttcccttctcctgaaagttcttgtggcggaggtggttctgggggcgggggaagcggaggaggggggagtatcgcagaactgccacctaaagtcagcgtctttgtgcctccccgtgatggcttttttggcaacccccggaagagtaaactaatatgccaggccacaggatttagtccgcggcagattcaggtgtcttggctccgggagggaaaacaggtcggaagtggagtgaccacagaccaagtgcaggccgaggctaaggagagtggtcctacaacttacaaggttacaagtaccttgacaattaaagagtccgattggctgggacagagtatgtttacatgtagagtggaccaccgtggtctcactttccagcagaatgctagttcaatgtgcgttccggatcaggataccgcaattagagtgttcgccatccctccctcttttgcttccatcttcctgaccaagagcaccaagcttacatgcttggttaccgatcttacaacctatgattctgtcactatctcctggacacgccagaacggggaggccgtgaagacacatactaatatcagcgagagccatcccaacgctacattctcagcagtgggcgaggcctctatctgcgaagacgactggaattcaggggaacggttcacgtgtactgtcacccatacagatctgccttcccctcttaagcagaccatttcacgaccgaaaggggtggctcttcaccggcccgacgtgtacctactcccacctgcccgtgaacaactgaatctgcgcgagagcgctaccattacctgcctggtaaccggatttagtcctgcagacgtctttgttcagtggatgcaacgcggccagcccctctcccctgagaagtatgtgactagtgcccctatgcccgaaccccaggctcctggcagatattttgcccatagcatcctgacagtatcagaagaggagtggaacactggtgaaacctatacttgcgtggtggcacacgaggctttgccgaaccgcgtcacagaacgaaccgttgacaaaagcacaggtaagcccaccctgtacaatgtcagcctcgtaatgtctgatacagcaggtacctgttat

SEQ ID NO: 61 - protein, 445 aa, synthetic construct
  source 1..445: note = TCR D046C1a wt, connecting peptide linker, Cu3-4/amino acid sequence
KNEVEQSPQNLTAQEGEFITINCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 62 - DNA, 1335 bp, synthetic construct
  source 1..1335: mol_type = other DNA; note = TCR D046C1a wt, connecting peptide linker, Cu3-4/nucleotide sequence
aagaatgaagtggagcaatcacctcaaaacctaaccgctcaggagggtgaattcatcaccatcaactgtagctactccgtcggcatctccgcactgcattggctccagcagcatcctggaggtggcatcgtcagcctcttcatgctgtcaagcggtaagaaaaagcatgggaggctgatcgccaccatcaatattcaggaaaaacacagcagtttgcacattacagcttcccacccccgtgactctgccgtgtacatctgcgctgctgacccttctgacagttggggcaaattgcagttcggcgctggcacccaggtcgttgtgaccccggacattcaaaaccccgatccggctgtctaccaactacgagactctaagagctctgacaaatccgtgtgcctttttaccgacttcgatagccaaacaaacgtgtctcagagcaaggacagcgacgtttatatcacagacaagactgtattggacatgcgctccatggacttcaagtccaatagcgctgttgcttggtccaataaaagtgatttcgcttgtgccaacgcttttaacaactctatcattcctgaggacacctttttcccttctcctgaaagttcttgtgatgttaagctggttgaaaagtccatccgagtattcgctatcccacccagtttcgcctctatcttccttaccaaatctactaaactgacatgcttggtcactgatctaaccacttacgattctgtgacaatttcctggacaaggcagaacggagaggcagtaaagactcatacgaatatttcagaatcccatcccaacgccacgttttccgccgttggcgaggcctccatttgtgaggacgattggaatagcggagagcgctttacctgcacagtgacgcatacagatctgcctagcccactcaagcagactatctctcgcccaaagggggtggctctgcataggcctgacgtatatctccttccacccgctcgagagcagctgaacttgagagaatccgcaactattacttgcttggtgacgggcttttctcccgccgatgtcttcgtgcagtggatgcagcgtggacagcctctctctcctgagaaatacgtaacatctgcccccatgcccgagcctcaggctcctggtcgttattttgctcacagtatcctgaccgtcagcgaagaggaatggaacactggagagacctacacctgtgtcgtggctcatgaagctctgcctaacagggtcaccgagcgcaccgtggacaaaagcactggcaaaccgaccctgtataacgtgtcactcgtgatgagcgacacagccggcacttgctat

SEQ ID NO: 63 - protein, 452 aa, synthetic construct
  source 1..452: note = TCR D046C1a wt, G-S linker, Cu3-4/amino acid sequence
KNEVEQSPQNLTAQEGEFITINCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCGGGGSGGGGSGGGGSIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 64 - DNA, 1356 bp, synthetic construct
  source 1..1356: mol_type = other DNA; note = TCR D046C1a wt, G-S linker, Cu3-4/nucleotide sequence
aagaatgaagtggagcaatcacctcaaaacctaaccgctcaggagggtgaattcatcaccatcaactgtagctactccgtcggcatctccgcactgcattggctccagcagcatcctggaggtggcatcgtcagcctcttcatgctgtcaagcggtaagaaaaagcatgggaggctgatcgccaccatcaatattcaggaaaaacacagcagtttgcacattacagcttcccacccccgtgactctgccgtgtacatctgcgctgctgacccttctgacagttggggcaaattgcagttcggcgctggcacccaggtcgttgtgaccccggacattcaaaaccccgatccggctgtctaccaactacgagactctaagagctctgacaaatccgtgtgcctttttaccgacttcgatagccaaacaaacgtgtctcagagcaaggacagcgacgtttatatcacagacaagactgtattggacatgcgctccatggacttcaagtccaatagcgctgttgcttggtccaataaaagtgatttcgcttgtgccaacgcttttaacaactctatcattcctgaggacacctttttcccttctcctgaaagttcttgtggcggaggtggttctgggggcgggggaagcggaggaggggggagtatccgagtattcgctatcccacccagtttcgcctctatcttccttaccaaatctactaaactgacatgcttggtcactgatctaaccacttacgattctgtgacaatttcctggacaaggcagaacggagaggcagtaaagactcatacgaatatttcagaatcccatcccaacgccacgttttccgccgttggcgaggcctccatttgtgaggacgattggaatagcggagagcgctttacctgcacagtgacgcatacagatctgcctagcccactcaagcagactatctctcgcccaaagggggtggctctgcataggcctgacgtatatctccttccacccgctcgagagcagctgaacttgagagaatccgcaactattacttgcttggtgacgggcttttctcccgccgatgtcttcgtgcagtggatgcagcgtggacagcctctctctcctgagaaatacgtaacatctgcccccatgcccgagcctcaggctcctggtcgttattttgctcacagtatcctgaccgtcagcgaagaggaatggaacactggagagacctacacctgtgtcgtggctcatgaagctctgcctaacagggtcaccgagcgcaccgtggacaaaagcactggcaaaccgaccctgtataacgtgtcactcgtgatgagcgacacagccggcacttgctat

SEQ ID NO: 65 - protein, 220 aa, synthetic construct
  source 1..220: note = PDL1 ECD
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 66 - protein, 114 aa, synthetic construct
  source 1..114: note = PDL1 D1
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA

SEQ ID NO: 67 - protein, 372 aa, synthetic construct
  source 1..372: note = Jch-(G4S)3-PDL1ECD amino acid sequence
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 68 - protein, 266 aa, synthetic construct
  source 1..266: note = Jch-(G4S)3-PDL1D1 amino acid sequence
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA

SEQ ID NO: 69 - protein, 553 aa, synthetic construct
  source 1..553: note = TCR-IgM
KNEVEQSPQNLTAQEGEFITIQCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKCVLDMRSMDFKSNSAVAWSQKSDFACANAFQNSIIPEDTFFPSPESSCIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 70 - protein, 163 aa, synthetic construct
  source 1..163: note = Jch-Flag-His8
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSDYKDDDDKHHHHHHHH

SEQ ID NO: 71 - protein, 440 aa, synthetic construct
  source 1..440: note = TCR D046C1 alpha chain-IgG
KNEVEQSPQNLTAQEGEFITIQCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKCVLDMRSMDFKSNSAVAWSQKSDFACANAFQNSIIPEDTFFPSPESSCDVKLVEKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 10 aa, synthetic construct
  source 1..10: note = ELA peptide
ELAGIGILTV

SEQ ID NO: 73 - protein, 418 aa, synthetic construct
  source 1..418: note = Anti-CD3scFv-Linker-Jch-Flag-His
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSDYKDDDDKHHHHHHHH

SEQ ID NO: 74 - protein, 408 aa, synthetic construct
  source 1..408: note = PD-L1-ECD-Jch-Flag-His
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGGGGSGGGGSGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSDYKDDDDKHHHHHHHH

SEQ ID NO: 75 - protein, 8 aa, synthetic construct
  source 1..8: note = His8 tag
HHHHHHHH

SEQ ID NO: 76 - protein, 100 aa, synthetic construct [sequence omitted]

SEQ ID NO: 77 - protein, 318 aa, synthetic construct
  source 1..318: note = HLA-A*02 soluble-Avi tag-His6-Flag
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSGSAGGGLNDIFEAQKIEWHEGSTGHHHHHHDYKDDDDK

SEQ ID NO: 78 - protein, 348 aa, synthetic construct
  source 1..348: note = IgM Cm2-Cm3-Cm4-TP L310D
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDDPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 79 - protein, 232 aa, synthetic construct
  source 1..232: note = IgM Cm3-Cm4-TP L310D
IRVFAIPPSFASIFLTKSTKLTCLVTDLTTYDSVTISWTRQNGEAVKTHTNISESHPNATFSAVGEASICEDDWNSGERFTCTVTHTDDPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 80 - protein, 348 aa, synthetic construct
  source 1..348: note = IgM Cm2-Cm3-Cm4-TP P311A, P313S
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 81 - protein, 232 aa, synthetic construct
  source 1..232: note = IgM Cm3-Cm4-TP P311A, P313S
IRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 82 - protein, 348 aa, synthetic construct
  source 1..348: note = IgM Cm2-Cm3-Cm4-TP P311G
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLGSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 83 - protein, 232 aa, synthetic construct
  source 1..232: note = IgM Cm3-Cm4-TP P311G
IRVFAIPPSFASIFLTKSTKLTCLVTDLTTYDSVTISWTRQNGEAVKTHTNISESHPNATFSAVGEASICEDDWNSGERFTCTVTHTDLGSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 84 - protein, 553 aa, synthetic construct
  source 1..553: note = TCR D046C1 a chain-IgM2-4 comp mt (TRAV: N22Q; TRAC: T84C, N90Q, N109Q)(IgM P311A, P313S)
KNEVEQSPQNLTAQEGEFITIQCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKCVLDMRSMDFKSNSAVAWSQKSDFACANAFQNSIIPEDTFFPSPESSCIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 85 - protein, 437 aa, synthetic construct
  source 1..437: note = TCR D046C1 a chain-IgM3-4 comp mt (TRAV: N22Q; TRAC: T84C, N90Q, N109Q)(IgM P311A, P313S)
KNEVEQSPQNLTAQEGEFITIQCSYSVGISALHWLQQHPGGGIVSLFMLSSGKKKHGRLIATINIQEKHSSLHITASHPRDSAVYICAADPSDSWGKLQFGAGTQVVVTPDIQNPDPAVYQLRDSKSSDKSVCLFTDFDSQTNVSQSKDSDVYITDKCVLDMRSMDFKSNSAVAWSQKSDFACANAFQNSIIPEDTFFPSPESSCIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 86 - protein, 243 aa, synthetic construct
  source 1..243: note = AM-gp-beta (affinity matured gp100 TCR beta with deglycosylation and stabilizing mutations) (TRBC2: E11K, H15.1R, D35P, S45.5D, N85.6Q, S79C, C85.1A) (PS1421)
DGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVTEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 87 - protein, 552 aa, synthetic construct
  source 1..552: note = AM-gp-alpha-IgMCu2-4 (affinity matured gp100 TCR alpha with deglycosylation and stabilizing mutations fused to IgM Cu2-4) (TRAC: S21F, T37I, A103T, N22Q, TRAC: N90Q, N109Q, T84C) (PS1416)
AQQGEEDPQALSIQEGENATMQCSYKTSINNLQWYRQNSGRGLVHLILIRSNEREKHSGRLRVTLDTSKKSSSLLITASRAADTASYFCATDGSTPMQFGKGTRLSVIANIQKPDPAVYQLRDSKSSDKFVCLFTDFDSQINVSQSKDSDVYITDKCVLDMRSMDFKSNSAVAWSQKSDFTCANAFQNSIIPEDTFFPSPESSCIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 88 - protein, 552 aa, synthetic construct
  source 1..552: note = Par-gp-alpha-IgMCu2-4 (Parent gp100 TCR alpha with stabilizing mutations fused to IgM Cu2-4) (TRAC: S21F, T37I, A103T, T84C) (PS1465)
SQQGEEDPQALSIQEGENATMNCSYKTSINNLQWYRQNSGRGLVHLILIRSNEREKHSGRLRVTLDTSKKSSSLLITASRAADTASYFCATDGDTPLVFGKGTRLSVIANIQKPDPAVYQLRDSKSSDKFVCLFTDFDSQINVSQSKDSDVYITDKCVLDMRSMDFKSNSAVAWSNKSDFTCANAFNNSIIPEDTFFPSPESSCIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 89 - protein, 243 aa, synthetic construct
  source 1..243: note = Par-gp-beta (Parent gp100 TCR beta with stabilizing mutations) (TRBC2: E11K, H15.1R, D35P, S45.5D, S79C, C85.1A) (PS1503)
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIGGPYEQYFGPGTRLTVTEDLKNVFPPEVAVFEPSKAEISRTQKATLVCLATGFYPPHVELSWWVNGKEVHDGVCTDPQPLKEQPALNDSRYALSSRLRVSATFWQDPRNHFRCQVQFYGLSENDEWTQDRAKPVTQIVSAEAWGRADC

SEQ ID NO: 90 - protein, 453 aa, synthetic construct
  source 1..453: note = Par-gp-alpha-g1Hn-IgMCu3-4 (Parent gp100 TCR alpha with stabilizing mutations fused to IgG1Hinge-IgMCu3-4) (TRAC: S21F, T37I, A103T, T84C) (PS1638)
SQQGEEDPQALSIQEGENATMNCSYKTSINNLQWYRQNSGRGLVHLILIRSNEREKHSGRLRVTLDTSKKSSSLLITASRAADTASYFCATDGDTPLVFGKGTRLSVIANIQKPDPAVYQLRDSKSSDKFVCLFTDFDSQINVSQSKDSDVYITDKCVLDMRSMDFKSNSAVAWSNKSDFTCANAFNNSIIPEDTFFPSPESSCDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 91 - protein, 453 aa, synthetic construct
  source 1..453: note = AM-gp-alpha-g1Hn-IgMCu3-4 (affinity matured gp100 TCR alpha with deglycosylation and stabilizing mutations fused to IgG1Hinge-IgMCu3-4) (TRAC: S21F, T37I, A103T) (TRAV: N22Q, TRAC: N90Q, N109Q) (Disulfile stabilized TRAC: T84C) (PS1632)
AQQGEEDPQALSIQEGENATMQCSYKTSINNLQWYRQNSGRGLVHLILIRSNEREKHSGRLRVTLDTSKKSSSLLITASRAADTASYFCATDGSTPMQFGKGTRLSVIANIQKPDPAVYQLRDSKSSDKFVCLFTDFDSQINVSQSKDSDVYITDKCVLDMRSMDFKSNSAVAWSQKSDFTCANAFQNSIIPEDTFFPSPESSCDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 92 - protein, 521 aa, synthetic construct
  source 1..521: note = U1v9-AM-gp-beta (scFv-UCHT1v9(DtoA)-affinity matured-TCR-beta with deglycosylation and stabilizing mutations + tags) (TRBC2: E11K, H15.1R, D35P, S45.5D), (TRBC2: N85.6Q), (TRBC2: S79C, C85.1A) (PL373_PS1364)
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVTEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGSDYKDDDDKLEHHHHHHHH

SEQ ID NO: 93 - protein, 204 aa, synthetic construct
  source 1..204: note = AM-gp-alpha (affinity matured gp100 TCR alpha with deglycosylation and stabilizing mutations) (TRAC: S21F, T37I, A103T), (TRAV: N22Q, TRAC: N90Q, N109Q), (Disulfide stabilized TRAC: T84C) (PL373_PS1365)
AQQGEEDPQALSIQEGENATMQCSYKTSINNLQWYRQNSGRGLVHLILIRSNEREKHSGRLRVTLDTSKKSSSLLITASRAADTASYFCATDGSTPMQFGKGTRLSVIANIQKPDPAVYQLRDSKSSDKFVCLFTDFDSQINVSQSKDSDVYITDKCVLDMRSMDFKSNSAVAWSQKSDFTCANAFQNSIIPEDTFFPSPESSC

SEQ ID NO: 94 - protein, 492 aa, synthetic construct
  source 1..492: note = Par-gp-alpha-U1v9 (Parent gp100 TCR alpha with stabilizing mutations fused to scFv U1v9 (DtoA) + tags) (TRAC: S21F, T37I, A103T), (Disulfide stabilized with TRAC: T84C) (PL427_PS1463)Par-gp-alpha-U1v9 (Parent gp100 TCR alpha with stabilizing mutations fused to scFv U1v9 (DtoA) + tags) (TRAC: S21F, T37I, A103T), (Disulfide stabilized with TRAC: T84C) (PL427_PS1463)
SQQGEEDPQALSIQEGENATMNCSYKTSINNLQWYRQNSGRGLVHLILIRSNEREKHSGRLRVTLDTSKKSSSLLITASRAADTASYFCATDGDTPLVFGKGTRLSVIANIQKPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGSDYKDDDDKLEHHHHHHHH

SEQ ID NO: 95 - protein, 249 aa, synthetic construct
  source 1..249: note = hingeIgG-IgMCu3-4
DKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 96 - protein, 441 aa, synthetic construct
  source 1..441: note = Human Ig Jch with anti-CD3 (UCHT1v9(D1A)) at N-terminus + tags (PS1429)
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSDYKDDDDKHHHHHHHH

SEQ ID NO: 97 - protein, 441 aa, synthetic construct
  source 1..441: note = Human Ig Jch with anti-CD3 (UCHT1v9(D1A)) at C-terminus + tags
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSDYKDDDDKHHHHHHHH

SEQ ID NO: 98 - protein, 9 aa, synthetic construct
  source 1..9: note = gp100pep Peptide
YLEPGPVTA

SEQ ID NO: 99 - protein, 392 aa, synthetic construct
  source 1..392: note = Jch-OKT3
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIK

SEQ ID NO: 100 - protein, 392 aa, synthetic construct
  source 1..392: note = OKT3-Jch
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 101 - protein, 253 aa, synthetic construct
  source 1..253: note = U1v9(D1A) scFv
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 102 - protein, 501 aa, synthetic construct
  source 1..501: note = U1v9-AM-gp-beta (scFv-UCHT1v9(DtoA)-affinity matured-TCR-beta with deglycosylation and stabilizing mutations) (TRBC2: E11K, H15.1R, D35P, S45.5D), (TRBC2: N85.6Q), (TRBC2: S79C, C85.1A) (PL373_PS1364)
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVTEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 103 - protein, 472 aa, synthetic construct
  source 1..472: note = Par-gp-alpha-U1v9 (Parent gp100 TCR alpha with stabilizing mutations fused to scFv U1v9 (DtoA)) (TRAC: S21F, T37I, A103T), (Disulfide stabilized with TRAC: T84C) (PL427_PS1463)
SQQGEEDPQALSIQEGENATMNCSYKTSINNLQWYRQNSGRGLVHLILIRSNEREKHSGRLRVTLDTSKKSSSLLITASRAADTASYFCATDGDTPLVFGKGTRLSVIANIQKPDPAVYQLRDSKSSDKFVCLFTDFDSQINVSQSKDSDVYITDKCVLDMRSMDFKSNSAVAWSNKSDFTCANAFNNSIIPEDTFFPSPESSCGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 104 - protein, 415 aa, synthetic construct
  source 1..415: note = Human Ig J chain with anti-CD3 (UCHT1v9(D1A)) at N-terminus
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 105 - protein, 415 aa, synthetic construct
  source 1..415: note = Human Ig J chain with anti-CD3 (UCHT1v9(D1A)) at C-terminus
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 106 - protein, 9 aa, synthetic construct
  source 1..9: note = heteroclitic gp100 peptide
YLEPGPVTV

(19) United States
(12) Patent Application Publication
(10) Pub. No.: US 2025/0326844 A1
(43) Pub. Date: Oct. 23, 2025

(54) T CELL RECEPTOR MULTIMERS AND USES THEREOF

(71) Applicant: REPERTOIRE IMMUNE MEDICINES, INC., Cambridge, MA (US)

(72) Inventors: Thomas J. MALIA, Cambridge, MA (US); Joanna SWAIN, Cambridge, MA (US); Andrew G. HAMEL, Cambridge, MA (US)

(21) Appl. No.: 18/854,898

(22) PCT Filed: Apr. 7, 2023
(86) PCT No.: PCT/US2023/065551

§ 371 Date: Oct. 7, 2024

Related U.S. Application Data
(60) Provisional application No. 63/328,719, filed on Apr. 7, 2022.

(51) Int. Cl. C07K 16/28; A61K 38/00; A61P 37/04; C07K 14/725; G01N 33/569; G01N 33/68

(52) U.S. Cl. CPC C07K 16/2809; A61P 37/04; C07K 14/7051; G01N 33/56966; G01N 33/6872; A61K 38/00; C07K 2317/622; C07K 2317/92; C07K 2319/30; G01N 2333/70539

(57) ABSTRACT

T cell receptor (TCR) multimers are provided in which a binding moiety from a T cell receptor variable region is attached to a multimerization moiety from IgM or IgA to thereby create a multivalent TCR multimer composition. The TCR multimers can be used, for example, to determine the binding specificity of the TCR-derived binding moiety, such as for particular MHC-peptide complexes. The multimers can also be used, for example, to modulate an immune response in a subject by administering the multimer to the subject. Methods of making the TCR multimers are also provided.

### CROSS-REFERENCE TO RELATED APPLICATIONS

This PCT application claims the benefit of U.S. Provisional Application No. 63/328,719, filed Apr. 7, 2022 which is incorporated herein by reference in its entirety.

### REFERENCE TO SEQUENCE LISTING SUBMITTED ELECTRONICALLY

The content of the electronically submitted sequence listing (Name: 4985_029PC01_Seqlisting_ST26; Size: 143,139 bytes; and Date of Creation: Apr. 7, 2023) is herein incorporated by reference in its entirety.

### BACKGROUND

T cells play a central role in generating, sustaining, and modulating immune responses, initiated by the binding of their antigen-specific T cell receptors (TCRs) to antigenic epitopes presented on Major Histocompatibility Complex (MHC) molecules (peptide-MHC, or pMHC). Given their importance in immune responses, determining which TCRs bind to which pMHC complexes is of great interest, for research, diagnostic, and therapeutic purposes. Moreover, a variety of adoptive T cell therapies, in which T cells are administered for therapeutic purposes, are being explored, such as for cancer treatment.

Examples of T cell therapies approaches include tumor infiltrating lymphocyte (TIL) therapy, engineered T cell receptor (TCR) therapy and chimeric antigen receptor (CAR) T cell therapy. Other T-cell mediated therapies aim to induce responses in T cells by administration of molecules that can block or activate T cells, such as agonistic or antagonistic antibodies to immune checkpoint proteins in treatment of a disease. Another approach that has been used is to engineer bispecific agonists that target TCRs and T cell receptors by bringing a receptor agonist to the TCR complex at a target cell-T cell interface to activate downstream signaling and impact (e.g., downregulate) T cell behavior.

While these approaches represent great achievement and advancement, each of these has one or more limitations on efficacy, safety, tolerability, etc.

Accordingly, despite the efforts made to date, there remains a need for additional agents and methods for determining TCR-pMHC interactions as well as for compositions for use in treating subjects in need of specific and selective modulation of T cell-mediated responses.

### SUMMARY

The present disclosure provides technologies comprising TCR multimers. In some aspects, the T cell receptor (TCR) multimers comprise a TCR moiety and a multimerization moiety, wherein the TCR moiety comprises an antigen binding variable (V) region of a TCR operatively linked to a multimerization moiety from an IgM or IgA molecule. In some embodiments, the multimerization moiety comprises at least one C region from IgM or IgA, as well as a tailpiece region from IgM or IgA, such that the fusion molecule forms multimers. In other embodiments, the multimerization moiety can comprise the tailpiece region of IgM or IgA alone. In some embodiments, the variable (V) regions comprise one or more of α,β, γ, or δ regions. For example, in certain embodiments, the variable region comprises an α chain V region, a β chain V region, a γ chain V region or a δ chain V region. Such TCR multimers in accordance with the present disclosure are multivalent and soluble. As disclosed herein, a combination of the antigen specificity of a TCR and the multi-valency of IgM or IgA provides a TCR-IgM or TCR-IgA fusion molecule with high avidity toward a specific peptide/MHC (pMHC) complex. Still further, the multimers can further comprise an effector moiety to confer additional functionality (e.g., T cell recruitment, redirection, elimination, etc.). Thus, in some embodiments, TCR multimers provided by the present disclosure can be used as therapeutic agents, in vivo or ex vivo, for treatment of a subject in need of immunomodulation. For example, in some embodiments, such TCR multimers may be used to treat cancers with known pMHC drivers, exploiting specificity of TCR/pMHC interactions, and thereby offering a specific and high avidity alternative to known approaches such as e.g., adoptive T cell transfer or chimeric antigen receptor (CAR)-based therapy. In other embodiments, multimers of the disclosure can be used for detecting TCR/pMHC interactions, e.g., in in vitro assays.

Accordingly, in one aspect, the disclosure provides T cell receptor (TCR) multimers comprising a TCR moiety comprising at least one variable (V) region, operatively linked to a multimerization moiety. In some such embodiments, the variable region comprises an α chain V region, a β chain V region, a γ chain V region or a δ chain V region. In certain embodiments, the TCR moiety comprises at least two TCR variable regions. In some embodiments, the TCR moiety comprises α chain V region and a β chain V region; or a γ chain V region and a δ chain V region.

In some embodiments, the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region. In some such embodiments, the multimerization moiety comprises an IgM Cμ4 constant region and an IgM tailpiece region and further comprises an IgM Cμ3 constant region. In some embodiments, the multimerization moiety comprises an IgM Cμ4 constant region and an IgM tailpiece region and further comprises an IgM Cμ2 constant region. In other embodiments, the multimerization moiety comprises an IgM Cμ4 constant region and an IgM tailpiece region and further comprises an IgM Cμ1 constant region. In still other embodiments, the multimerization moiety comprises an IgM Cμ4 constant region and an IgM tailpiece region and further comprises IgM Cμ2 and Cμ3 constant regions. In some embodiments, the multimerization moiety comprises an IgM Cμ4 constant region and an IgM tailpiece region and further comprises IgM Cμ1 and Cμ3 constant regions. In some embodiments, the multimerization moiety comprises an IgM Cμ4 constant region and an IgM tailpiece region and further comprises IgM Cμ1 and Cμ2 constant regions. In other embodiments, the multimerization moiety comprises an IgM Cμ4 constant region and an IgM tailpiece region and further comprises IgM Cμ1, Cμ2 and Cμ3 constant regions.

In certain embodiments, the multimerization moiety comprises an IgA Cα3 constant region and an IgA tailpiece region and further comprises an IgA Cα2 constant region. In some embodiments, the multimerization moiety comprises an IgA Cα3 constant region and an IgA tailpiece region and further comprises an IgA Cα1 constant region. In other embodiments, the multimerization moiety comprises an IgA Cα3 constant region and an IgA tailpiece region and further comprises IgA Cα1 and Cα2 constant regions.

In certain embodiments of the multimerization moiety, the IgM or IgA tailpiece region alone is used for multimerization, without inclusion of any immunoglobulin C regions.

In other aspects, the disclosure provides T cell receptor (TCR) multimers comprising a TCR moiety operatively linked to a multimerization moiety, wherein the TCR moiety comprises at least one variable (V) region; and (b) the multimerization moiety comprises an IgM tailpiece region or an IgA tailpiece region. In some embodiments, the variable region comprises an α chain V region, a β chain V region, a γ chain V region or a δ chain V region.

In certain embodiments, the multimerization moiety comprises the IgM tailpiece region. In other embodiments, the multimerization moiety comprises the IgA tailpiece region.

In some embodiments, the TCR multimer comprises TCR constant (C) regions as well as TCR V regions. For example, in some embodiments, the TCR moiety comprises TCR α and β chain V regions and further comprises TCR α and β chain constant (C) regions. In another embodiment, the TCR moiety comprises TCR γ and δ chain V regions and further comprises TCR γ and δ constant (C) regions.

In certain embodiments, the TCR multimer further comprises an immunoglobulin J chain.

In certain embodiments, the TCR multimer is soluble. In other embodiments, the TCR multimer can be fixed to a solid support (e.g., a plate or a bead).

In certain embodiments, the TCR moiety is a single chain TCR (e.g., single chain (α/β V regions or γ/δ V regions).

In various embodiments, the TCR multimer is a dimer, a trimer, a tetramer, a pentamer, or a hexamer. In some embodiments, the TCR multimer is in a composition comprising a mixture of at least two multimers selected from the group consisting of dimers, trimers, tetramers, pentamers, and hexamers.

In some embodiments the TCR moiety of the TCR multimer is operatively linked to the multimerization moiety through the TCR α chain or the TCR γ chain. In other embodiments, the TCR moiety is operatively linked to the multimerization moiety through the TCR β chain or the TCR δ chain.

In certain embodiments, the TCR moiety and the multimerization moiety are separated by a linker. In some such embodiments, the linker is a flexible linker, a rigid linker, or a semi-rigid linkers.

In some embodiments, the TCR multimer comprises an immunoglobulin J chain and the TCR multimer further comprises an effector moiety operatively linked to the J chain. In other embodiments, the TCR multimer comprises an effector moiety operatively linked to the multimerization moiety.

In certain embodiments, the effector moiety comprises a checkpoint protein agonist. In some such embodiments, the checkpoint protein agonist comprises a PD-L1 extracellular domain or domain 1, an anti-PD1 agonist antibody, an anti-CTLA4 agonist antibody, an anti-Lag3 agonist antibody, an anti-TIM3 agonist antibody, an anti-TIGIT agonist antibody, an anti-LILRB1 agonist antibody, an anti-LILRB2 agonist antibody, an HLA-G, or any portion or antigen-binding fragment thereof.

In other embodiments, the effector moiety comprises an immune cell engaging agent. In some such embodiments, the immune cell engaging agent comprises an anti-CD3 antibody, an anti-NKp46 antibody, an anti-CD16a antibody, an anti-CD56 antibody, an anti-NKG2D antibody, an anti-NKp30 antibody, an anti-CD64 antibody, or any portion or antigen-binding fragments thereof. In some embodiments, the immune cell engaging agent comprises an anti-CD3 antibody. In some embodiments, the anti-CD3 antibody comprises an anti-CD3 scFv antibody.

In other embodiments, the effector moiety comprises a cell-death inducing agent. In some such embodiments, the cell-death-inducing agent comprises a tubulin inhibitor, a DNA damaging agent, FasL, anti-Fas agonist antibody or any portion or antigen-binding fragment thereof.

In certain embodiments, the effector moiety is attached to the TCR multimer through a linker. In some embodiments, the linker is positioned between the J chain and the effector moiety. In other embodiments, the linker is positioned between the multimerization moiety and the effector moiety. In some embodiments, the linker is a flexible linker, a rigid linker, or a semi-rigid linker.

In certain embodiments, the TCR multimer further comprises one or more heterologous functional domains (e.g., a moiety for half-life extension, a cytokine, a cytokine receptor).

In some embodiments, the TCR multimer is encoded by one or more isolated nucleic acid molecules. In some embodiments, an expression vector comprises the one or more isolated nucleic acid molecules.

TCR multimers provided herein can be prepared by chemical conjugation of their components, but more typically are prepared recombinantly using expression vectors encoding the multimer components introduced into host cells. Accordingly, in another aspect, the disclosure provides isolated nucleic acids encoding subunits of the TCR multimers.

In certain embodiments, the disclosure provides an isolated nucleic acid molecule encoding a T cell receptor (TCR) moiety operatively linked to a multimerization moiety. In some embodiments, TCR moiety comprises at least one variable (V) region; and the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region. In some embodiments, the V region comprises an α chain V region, a β chain V region, a γ chain V region or a δ chain V region. In certain embodiments, the TCR moiety comprises at least two V regions. In some such embodiments, the TCR moiety comprises an α chain V region and a β chain V region; or a γ chain V region and a δ chain V region.

In some embodiments of the isolated nucleic acid, the multimerization moiety comprises an IgM Cμ4 constant region and an IgM tailpiece region and further comprises an IgM Cμ3 constant region. In some embodiments, the multimerization moiety comprises an IgM Cμ4 constant region and an IgM tailpiece region and further comprises an IgM Cμ2 constant region. In some embodiments, the multimerization moiety comprises an IgM Cμ4 constant region and an IgM tailpiece region and further comprises an IgM Cμ1 constant region. In some embodiments, the multimerization moiety comprises an IgM Cμ4 constant region and an IgM tailpiece region and further comprises IgM Cμ2 and Cμ3 constant regions. In some embodiments, the multimerization moiety comprises an IgM Cμ4 constant region and an IgM tailpiece region and further comprises IgM Cμ1 and Cμ3 constant regions. In some embodiments, the multimerization moiety comprises an IgM Cμ4 constant region and an IgM tailpiece region and further comprises IgM Cμ1 and Cμ2 constant regions. In some embodiments, the multimerization moiety comprises an IgM Cμ4 constant region and an IgM tailpiece region and further comprises IgM Cμ1, Cμ2 and Cμ3 constant regions.

In certain embodiments of the isolated nucleic acid, the multimerization moiety comprises an IgA Cα3 constant region and an IgA tailpiece region and further comprises an IgA Cα2 constant region. In some embodiments, the multimerization moiety comprises an IgA Cα3 constant region and an IgA tailpiece region and further comprises an IgA Cα1 constant region. In some embodiments, the multimerization moiety comprises an IgA Cα3 constant region and an IgA tailpiece region and further comprises IgA Cal and Cα2 constant regions.

In other embodiments of the isolated nucleic acid encoding the TCR multimer, the multimerization moiety comprises the IgM or IgA tailpiece region alone, without inclusion of any immunoglobulin C regions. Thus, in another aspect, the disclosure provides isolated nucleic acid molecules encoding a T cell receptor (TCR) multimer comprising a TCR moiety operatively linked to a multimerization moiety. In some embodiments, the TCR moiety comprises at least one variable (V) region and the multimerization moiety comprises an IgM tailpiece region or an IgA tailpiece region. In some embodiments, the V region comprises an α chain V region, a β chain V region, a γ chain V region or a δ chain V region. In certain embodiments, the TCR moiety comprises at least two TCR variable regions. In some such embodiments, the TCR moiety comprises an α chain V region and a β chain V region; or a γ chain V region and a δ chain V region.

In some embodiments, the multimerization moiety comprises the IgM tailpiece region. In other embodiments, the multimerization moiety comprises the IgA tailpiece region.

In some embodiments of the isolated nucleic acid, the TCR moiety further comprises a TCR chain constant (C) region (e.g., TCR α chain V and C regions, TCR β chain V and C regions, TCR γ chain V and C regions or TCR δ chain V and C regions).

In some embodiments of the isolated nucleic acid, the TCR moiety and the multimerization moiety are separated by a linker. In some embodiments, the linker is a flexible linker, a rigid linker, or a semi-rigid linker.

In yet another aspect, the disclosure provides certain expression vectors and host cells comprising isolated nucleic acids provided herein. Accordingly, in some embodiments, an isolated nucleic acid encoding a TCR V region-IgM/IgA C region fusion can be incorporated into an expression vector and the expression vector can be introduced into a host cell to thereby express the fusion protein. In some embodiments, the host cell comprises an expression vector encoding a TCR moiety comprising a TCR V region linked to the IgM or IgA C region and a separate expression vector encoding the cognate TCR V region chain that pairs with the TCR moiety (e.g., resulting in α/β pairs or γ/δ pairs in the host cell). In some embodiments, the expression vector encoding the cognate TCR V region chain is operatively linked to a TCR C region chain.

In certain embodiments, the host cell further comprises a nucleic acid molecule or expression vector encoding an immunoglobulin J chain. In some embodiments, the immunoglobulin J chain is operatively linked to an effector moiety. In some embodiments, where the nucleic acid molecule or expression vector encoding the immunoglobulin J chain is part of the same nucleic acid molecule or expression vector encoding the cognate TCR V region or is a separate expression vector.

In some embodiments, the host cell comprises an expression vector comprising an effector moiety.

In some embodiments, the effector moiety comprises a checkpoint protein agonist. In some such embodiments, the checkpoint protein agonist comprises a PD-L1 extracellular domain or domain 1, an anti-PD1 agonist antibody, an anti-CTLA4 agonist antibody, an anti-Lag3 agonist antibody, an anti-TIM3 agonist antibody, an anti-TIGIT agonist antibody, an anti-LILRB1 agonist antibody, an anti-LILRB2 agonist antibody, or any portion or antigen-binding fragment thereof.

In other embodiments, the effector moiety comprises an immune cell engaging agent. In some such embodiments, the immune cell engaging agent comprises an anti-CD3 antibody, an anti-NKp46 antibody, an anti-CD16a antibody, an anti-CD56 antibody, an anti-NKG2D antibody, an anti-NKp30 antibody, an anti-CD64 antibody, or any portion or antigen-binding fragments thereof. In some embodiments, the effector moiety comprises an anti-CD3 antibody. In some embodiments, the anti-CD3 antibody comprises an anti-CD3 scFv antibody.

In still other embodiments, the effector moiety comprises a cell-death inducing agent. In some embodiments, the cell-death-inducing agent comprises a tubulin inhibitor, a DNA damaging agent, FasL, anti-Fas agonist antibody or any portion or antigen-binding fragment thereof.

Methods of preparing TCR multimers of the disclosure are also provided in which a host cell carrying expression vectors encoding the multimer components are cultured to thereby express the multimer. The methods can further comprise isolating the TCR multimer from the host cells or the host cell culture supernatant.

In yet another aspect, the disclosure pertains to a pharmaceutical composition comprising a TCR multimer of the disclosure and a pharmaceutically acceptable carrier. Kits comprising the TCR multimer packaged in a container with instructions for use in modulating an immune response are also provided.

In yet another aspect, the disclosure provides methods of detecting a peptide-MHC complex, the method comprising contacting a peptide-MHC complex with a TCR multimer of the disclosure and detecting binding of the TCR multimer to the peptide-MHC complex to thereby detect the peptide-MHC complex. In some embodiments, the peptide-MHC complex is on the surface of an antigen presenting cell (APC) and the TCR multimer is contacted with the APC.

In one aspect, the disclosure provides methods of modulating an immune response in a subject, the method comprising administering to the subject a TCR multimer of the disclosure such that an immune response is modulated in the subject.

In some embodiments, the subject has cancer and the immune response to the cancer is modulated. Accordingly, the disclosure also provides a method of treating cancer in a subject, the method comprising administering to the subject a TCR multimer of the disclosure, wherein the TCR moiety of the TCR multimer recognizes a cancer antigen of the subject's cancer. In some embodiments, the cancer is a hematological cancer. In some embodiments, the cancer is a solid tumor. In some embodiments, the present disclosure provides a method of treating cancer using an immunotherapy, the method comprising administering an effective amount of a TCR multimer in accordance with the present disclosure to a subject in need thereof.

In other embodiments, the subject has an infectious disease and the immune response to the infectious disease is modulated by administration of a multimer as provided herein. Accordingly, the disclosure also provides a method of treating an infectious disease caused by a pathogen in a subject, the method comprising administering to the subject a TCR multimer of the disclosure, wherein the TCR moiety of the TCR multimer recognizes a pathogen antigen of the subject's infectious disease. In some embodiments, the infectious disease is a viral infection.

In another embodiment, the disclosure provides a method of treating an autoimmune disease, the method comprising administering to the subject the TCR multimer of the disclosure, wherein autoimmune response is modulated.

In yet another embodiment, the disclosure provides a method of enhancing an immune response to a vaccine, the method comprising administering to the subject the vaccine and a TCR multimer of the disclosure, wherein the TCR moiety of the TCR multimer recognize an antigen of the vaccine.

In some embodiments, the disclosure provides uses of TCR multimers in the manufacture of a medicament for treating cancer, an infectious disease, and/or an autoimmune disease. In some such embodiments, the cancer is a hematological cancer or a solid tumor. In some embodiments, the autoimmune disease is characterized by the presence of one or more autoantigens.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### BRIEF DESCRIPTION OF THE FIGURES

A better understanding of the features and advantages of the present disclosure will be obtained by reference to the following detailed description that sets forth illustrative embodiments, in which the principles of the disclosure are utilized, and the accompanying drawings of which:

FIGS. 1A-1C are schematic representations of exemplary TCR multimers of the disclosure. In FIG. 1A, the TCR multimer comprises TCR variable and constant regions as the TCR moiety, IgM Cμ3 and Cμ4 constant regions and IgM tailpiece region in the multimerization moiety, as well as an immunoglobulin J chain. In FIG. 1B, the TCR multimer comprises TCR variable and constant regions as the TCR moiety, IgM Cμ2, Cμ3 and Cμ4 constant regions and IgM tailpiece region in the multimerization moiety, as well as an immunoglobulin J chain. In FIG. 1C the TCR multimer comprises TCR variable and constant regions as the TCR moiety, IgA Cα2 and Cα3 constant regions and IgA tailpiece region, as well as an immunoglobulin J chain. Disulfide bridges are indicated by dashed black lines.

FIGS. 2A-C are schematic representations of exemplary TCR multimers of the disclosure that comprise an effector moiety. In FIG. 2A, the TCR multimer comprises TCR variable and constant regions as the TCR moiety, IgM Cμ3 and Cμ4 constant regions and IgM tailpiece region in the multimerization moiety, as well as an immunoglobulin J chain linked to an effector. In FIG. 2B, the TCR multimer comprises TCR variable and constant regions as the TCR moiety, IgM Cμ2, Cμ3 and Cμ4 constant regions and IgM tailpiece region in the multimerization moiety, an immunoglobulin J chain, and an effector moiety linked to the J chain. In FIG. 2C the TCR multimer comprises TCR variable and constant regions as the TCR moiety, IgA Cα2 and Cα3 constant regions and IgA tailpiece region in the multimerization moiety and an immunoglobulin J chain linked to an effector/targeting moiety (labelled “effector moiety”). Disulfide bridges are indicated by dashed black lines.

FIGS. 3A-3C are schematic representations of exemplary nucleic acid constructs used to recombinantly express a TCR multimer of the disclosure. Components of the multimer encoded by each nucleic acid are (P=Peptide, L=Linker, tp=tailpiece, J=J chain, EM=effector moiety T2A=T2A self-cleaving peptide). FIG. 3A is a schematic representation of three exemplary constructs encoding a TCRα subunit including a multimerization domain, a TCRβ subunit, and a J chain subunit. FIG. 3B is a schematic representation of three exemplary constructs encoding a TCRα subunit including a multimerization domain, a TCRβ subunit, and a J chain subunit operatively linked to an effector moiety or functional moiety. FIG. 3C is a schematic representation of two exemplary constructs encoding a TCRα subunit including a multimerization domain, and a TCRβ subunit separated by a T2A self cleaving peptide with a J chain subunit operatively linked to an effector moiety or functional moiety for tandem expression.

FIG. 4 is a schematic representation of an approach for assessing the ability of TCR-Ig-cytotoxin multimer for inducing targeted tumor cell death.

FIG. 5 is a schematic representation of an approach for assessing the ability of TCR-Ig-anti-CD3 multimer for redirecting T cells to kill tumor cells.

FIG. 6 illustrates a schematic representation of a process for assessing the ability of an exemplary pMHC-Ig-immunosuppressor to suppress the activity of target T cells, where (I) represents measurement of increased percent (%) live target cells, (II) represents monitoring reduced activation level of effector T cells by flow cytometry or by western blot, (III) represents measurement of reduced proliferation of antigen-specific T cells, and (IV) represents testing for reduced cytokine secretion (e.g., IFNγ, IL2, and/or TNFα).

FIGS. 7A-7B show results of a purity analysis from an exemplary overexpressed TCR-IgM multimer. FIG. 7A shows an analytical Size Exclusion Chromatogram (SEC) of purified D046C1-IgM protein. FIG. 7B shows a western blot probed with anti-IgM antibody and anti-Flag antibody under non-reduced/non-boiled (left panel) and reduced/boiled (right panel) conditions.

FIGS. 8A-8B shows results from a biolayer interferometry binding assay of exemplary TCR-IgM multimers (D046C1-IgM and D046C1-IgG) to biotinylated peptide-MHC complexes (bt-NLV/HLA-A2) at different concentrations. FIG. 8A shows a binding profile of D046C1-IgM on biotinylated NLV/HLA-A2 monomer and ELA/HLA-A2 monomer. FIG. 8B shows a binding profile of D046C1-IgG on biotinylated NLV/HLA-A2 monomer and ELA/HLA-A2 monomer.

FIG. 9A shows a western blot of an exemplary TCR-IgM/Effector-J-chain multimer with anti-IgM and anti-Flag under non-reduced/non-boiled (left panel) and reduced/boiled (right panel) condition. FIG. 9B shows an analytical SEC of purified D046C1-IgM/anti-CD3-J-chain (top panel) and D046C1-IgM/PD-L1-ECD-J-chain (bottom panel).

FIGS. 10A-10D show results from a biolayer interferometry binding assay of exemplary TCR-IgM multimers (Par-gp-IgM2-4/U1v9-J (PL421), anti-CD3scFv-Par-gp100 monomer (PL427), and anti-CD3scFv-AM-gp100 monomer (PL373)) to biotinylated peptide-MHC complexes (YLEPGPVTA-HLA-A*02:01 complex (MPL141) or an irrelevant-peptide-HLA-A*02:01 complex (MPL138)) at different concentrations. FIG. 10A shows a binding profile of Par-gp-IgM2-4/U1v9-J (PL421) binding to biotinylated YLEPGPVTA-HLA-A*02:01 complex. FIG. 10B shows a binding profile of anti-CD3scFv-Par-gp100 monomer (PL427) binding to biotinylated YLEPGPVTA-HLA-A*02:01 complex. FIG. 10C shows a binding profile of anti-CD3scFv-AM-gp100 monomer (PL373) binding to biotinylated YLEPGPVTA-HLA-A*02:01 complex. FIG. 10D shows a binding profile of Par-gp-IgM2-4/U1v9-J (PL421) binding irrelevant-peptide-HLA-A*02:01 complex.

FIGS. 11A-11E show results from a biolayer interferometry binding assay of exemplary TCR-IgM multimers (AM-gp-IgM2-4/OKT3-Jch (PL433), Par-gp-IgM3-4/Jch-OKT3 (PL456), AM-gp-IgM3-4/Jch-OKT3 (PL466), Par-gp-IgM2-4/Jch-OKT3 (PL436), and Par-gp-IgM3-4/OKT3-Jch (PL465)) to biotinylated peptide-MHC complexes (YLEPGPVTA-HLA-A*02:01 complex (MPL141)) at different concentrations. FIG. 11A shows a binding profile of AM-gp-IgM2-4/OKT3-Jch (PL433) binding to biotinylated YLEPGPVTA-HLA-A*02:01 complex. FIG. 11B shows a binding profile of Par-gp-IgM3-4/Jch-OKT3 (PL456) binding to biotinylated YLEPGPVTA-HLA-A*02:01 complex. FIG. 11C shows a binding profile of AM-gp-IgM3-4/Jch-OKT3 (PL466) binding to biotinylated YLEPGPVTA-HLA-A*02:01 complex complex. FIG. 11D shows a binding profile of Par-gp-IgM2-4/Jch-OKT3 (PL436) binding biotinylated YLEPGPVTA-HLA-A*02:01 complex. FIG. 11E shows a binding profile of Par-gp-IgM3-4/OKT3-Jch (PL465) binding biotinylated YLEPGPVTA-HLA-A*02:01 complex.

FIGS. 12A-12F show results from a biolayer interferometry binding assay of exemplary TCR-IgM multimers (AM-gp-IgM2-4/OKT3-Jch (PL433), Par-gp-IgM2-4/Jch-OKT3 (PL436), AM-gp-IgM2-4/Jch-OKT3 (PL434), Par-gp-IgM3-4/OKT3-Jch (PL465), Par-gp-IgM3-4/Jch-OKT3 (PL456), and AM-gp-IgM/UCHT1-J (PL420)) to biotinylated human CD3 epsilon/gamma at different concentrations. FIG. 12A shows a binding profile of AM-gp-IgM2-4/OKT3-Jch (PL433) binding to biotinylated human CD3 epsilon/gamma. FIG. 12B shows a binding profile of Par-gp-IgM2-4/Jch-OKT3 (PL436) binding to biotinylated human CD3 epsilon/gamma. FIG. 12C shows a binding profile of AM-gp-IgM2-4/Jch-OKT3 (PL434) binding to biotinylated human CD3 epsilon/gamma. FIG. 12D shows a binding profile of Par-gp-IgM3-4/OKT3-Jch (PL465) binding biotinylated human CD3 epsilon/gamma. FIG. 12E shows a binding profile of Par-gp-IgM3-4/Jch-OKT3 (PL456) binding biotinylated human CD3 epsilon/gamma. FIG. 12F shows a binding profile of AM-gp-IgM/UCHT1-J (PL420) binding biotinylated human CD3 epsilon/gamma.

FIG. 13A shows an SDS-PAGE gel under reducing conditions for Par-gp-IgM3-4/Jch-OKT3 (PL456), Par-gp-IgM3-4/OKT3-Jch (PL465), Par-gp-IgM2-4/OKT3-Jch (PL435), Par-gp-IgM2-4/U1v9-J (PL421), and AM-gp-IgM3-4/Jch-OKT3 (PL466). FIG. 13B shows a Western blot under reducing conditions probed with anti-IgM antibody (green) and anti-Flag antibody (red).

FIGS. 14A-14F show the results of a purity analysis from additional exemplaryoverexpressed TCR-IgM multimers of gp100par-IgM2-4/U1v9-J (PL421) (FIG. 14A), gp100par-IgM2-4/OKT3-Jch (PL435) (FIG. 14B), Teb-IgM3-4/Jch-OKT3 (PL466) (FIG. 14C), gp100par-IgM3-4/Jch-OKT3 (PL456) (FIG. 14D), gp100par-IgM2-4/Jch-OKT3 (PL436) (FIG. 14E), and gp100par-IgM3-4/OKT3-Jch (PL465) (FIG. 14F).

FIG. 15 shows ELISpot results of T cells mixed with heteroclitic gp100-peptide-loaded APCs at different concentrations of TCR IgM/anti-CD3 or monovalent TCR/anti-CD3.

### DETAILED DESCRIPTION

The present disclosure provides technologies such as compositions, methods of manufacturing, and methods of treatment comprising multivalent T cell receptor (TCR) multimers. As provided herein, each TCR multimer comprises a fusion of a TCR moiety and a multimerization moiety, wherein the TCR moiety comprises the antigen binding region of a TCR (α/β V regions or γ/δ V regions) operatively linked to a multimerization moiety that comprises at least one C region from an IgM or IgA molecule such that the fusion molecule forms multimers. The TCR multimers of the disclosure can further comprise an immunoglobulin J chain, which facilitates formation of certain forms of multimers (e.g., pentamers). Additionally, the TCR multimers can further comprise an effector moiety (e.g., anti-CD3), which can facilitate T cell redirection such as suppressing or killing, supporting, or otherwise influencing certain T cell populations to treat cancers, infectious diseases, and autoimmune diseases.

For example, in some embodiments, a TCR multimer comprising an effector moiety such as a TCR-IgM/A-αCD3 fusion molecule provided in accordance with the present disclosure can function by targeting and binding specific peptide/MHC molecules on the cell surface of antigen-presenting cells through the TCR moiety with high avidity, and subsequently recruiting effector T-cells via anti-CD3 binding to CD3 on the T-cell surface. TCR recruitment then results in the targeted killing of the antigen-presenting cell.

TCR-pMHC interaction affinity is known to be low (as low as μM), yet sufficient for precise biological activity. Enhancing the affinity of recombinant cell-expressed TCRs through affinity maturation has been attempted, but with deleterious effects in safety due to cross-reactivity. The present disclosure provides technologies that can overcome some of these deleterious safety features. For example, in some embodiments, by maintaining the native sequence of the TCR-CDRs, the TCR multimers of the disclosure are designed to have the same specificity for pMHC as a TCR on cells. Moreover, since the TCR multimers provided herein are multivalent, having a high number of TCR binding sites (e.g., ten for a pentameric TCR multimer of the disclosure) in close proximity facilitates highly avid interactions with pMHCs without increasing non-specific binding.

While not intending to be limited by mechanism, it is thought that the mechanism of action for TCR-Ig multimers without an effector moiety may be through complement-mediated cytotoxicity (CDC). Similarly, without being bound by any particular theory, for TCR-Ig multimers comprising an effector moiety, it is thought that the mechanism of action may be through TCR-mediated killing via TCR redirection and/or through CDC.

Various components and aspects of the disclosure are described in further detail in the subsections below.

### I. Definitions

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present disclosure that consist essentially of, or consist of, the recited processing steps.

In the disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method provided and described herein can be combined in a variety of ways without departing from the spirit and scope of the present disclosure and invention(s) herein, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of invention(s) provided, described, and depicted herein.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention(s) remain operable. Moreover, two or more steps or actions may be conducted simultaneously.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of any invention(s) unless claimed. No language in the specification should be construed as indicating any non-claimed element as essential to the practice of that provided by the present disclosure.

The terms “antigenic peptide”, “antigenic determinant” or “epitope” refer to a site on an antigen to which a T-cell receptor, an MHC molecule or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation and typically can include up to about 25 amino acids. Methods for determining what epitopes are bound by a given TCR or antibody (i.e., epitope mapping) are well known in the art and include, for example, immunoblotting and immunoprecipitation assays, wherein overlapping or contiguous peptides from the antigen are tested for reactivity with the given TCR or immunoglobulin. Methods of determining spatial conformation of epitopes include techniques in the art and those described herein, for example, x-ray crystallography, nuclear magnetic resonance, cryo-electron microscopy (cryo-EM), hydrogen deuterium exchange mass spectrometry (HDX-MS), and site-directed mutagenesis (see, e.g., EPITOPE MAPPING PROTOCOLS IN METHODS IN MOLECULAR BIOLOGY, Vol. 66, G. E. Morris, Ed. (1996)).

As used herein, the terms “antigen-presenting cell” or “APC” refer to a cell that mediate a cellular immune response by displaying an epitope on its outer surface, presented by a protein or protein complex such as, e.g., a major histocompatibility complex (MHC), or MHC-related protein such as CD1, that can present lipids to T cells, for recognition by an immune cell such as a T cell. APCs include professional APCs, such as dendritic cells, macrophages, Langerhans cells, and B cells, that express both class I and class II MHCs, and non-professional APCs (e.g., nucleated cells) that express only class I MHCs. APCs also include artificial APCs, such as cells (e.g., drosophila cells) engineered to express a MHC that presents a T cell epitope.

The term “avidity” as used herein, refers to the binding strength of as a function of the cooperative interactivity of multiple binding sites of a multivalent molecule (e.g., a soluble multivalent TCR multimer) with a target molecule. A number of technologies exist to characterize the avidity of molecular interactions including switchSENSE and surface plasmon resonance (Gjelstrup et al. (2012) J. IMMUNOL., 188:1292-1306); Vorup-Jensen (2012) ADV. DRUG. DELIV. REV., 64:1759-1781).

As used herein a “barcode”, also referred to as an oligonucleotide barcode, is a typically short nucleotide sequence (e.g., about 1, 2, 3, 4, 5, 6, 7, 8, 9, 10, 11, 12, 13, 14 or 15 nucleotides long or longer) that identifies a molecule to which it is conjugated. Barcodes can be used, for example, to identify molecules in a reaction mixture. Barcodes uniquely identify the molecule to which it is conjugated, for example, by performing reverse transcription using primers that each contain a “unique molecular identifier” barcode. In other embodiment, primers can be utilized that contain “molecular barcodes” unique to each molecule. The process of labeling a molecule with a barcode is referred to herein as “barcoding.” A “DNA barcode” is a DNA sequence used to identify a target molecule during DNA sequencing. In some embodiments, a library of DNA barcodes is generated randomly, for example, by assembling oligos in pools. In other embodiments, the library of DNA barcodes is rationally designed in silico and then manufactured.

“Binding affinity” generally refers to the strength of the sum total of noncovalent interactions between a single binding site of a molecule (e.g., a TCR variable region) and its binding partner. Unless indicated otherwise, as used herein, “binding affinity” refers to intrinsic binding affinity which reflects a 1:1 interaction between members of a binding pair (e.g., TCR and peptide-MHC). The affinity of a molecule X for its partner Y can generally be represented by the dissociation constant (Kd). For example, the Kd can be about 1 μM, 500 nM, 200 nM, 150 nM, 100 nM, 60 nM, 50 nM, 40 nM, 30 nM, 20 nM, 10 nM, 8 nM, 6 nM, 4 nM, 2 nM, 1 nM, or less than 1 nM. A variety of methods of measuring binding affinity are known in the art, any of which can be used for purposes of the present disclosure.

As used herein, the terms “carrier” refers to a material, composition or vehicle, such as a liquid or solid filler, diluent, excipient, solvent or encapsulating material, involved in carrying or transporting a pharmaceutical agent, or a pharmaceutically acceptable salt thereof, from one organ, or portion of the body, to another organ, or portion of the body. A “pharmaceutically acceptable carrier” includes any and all solvents, dispersion media, coatings, antibacterial and antifungal agents, isotonic and absorption delaying agents, and the like that are physiologically compatible.

As used herein, the term “cleavage site” or “cleavable moiety” refers to a site, a motif or sequence that is cleavable, such as by an enzyme (e.g., a protease) or by particular reaction conditions. In some embodiments, the cleavage moiety comprises a protein, e.g., enzymatic, cleavage site. In some embodiments, the cleavage moiety comprises a chemical cleavage site, e.g., through exposure to oxidation/reduction conditions, light/sound, temperature, pH, pressure, etc.

The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

As used herein, “effective amount” or “therapeutically-effective amount” refers to the amount of an active agent (e.g., a TCR multimer as provided herein) sufficient to effect beneficial or desired results. An effective amount can be administered in one or more administrations, applications or dosages and is not intended to be limited to a particular formulation or administration route.

As used herein, the term “expression construct” refers to a vector designed for gene expression, e.g., in a host cell. An expression vector promotes the expression (i.e., transcription/translation) of an encoded polypeptide (e.g., fusion polypeptide). Typically, the vector is a plasmid, although other suitable vectors, including viral and non-viral vectors are also encompassed by the term “expression construct.”

As used herein, an “IgM constant region” refers to all or a portion of the four constant region domains of immunoglobulin M, referred to as Cμ1, Cμ2, Cμ3 and Cμ4 (also used herein interchangeably with “Cμ1,” “Cμ2,” “Cμ3,” and “Cμ4,” respectively, or “Cm1,” “Cm2,” “Cm3,” and “Cm4,” respectively), (each approximately 110 amino acids), including alleles (including the four human IgM constant region alleles), variants and modified forms that retain the functional activity of the naturally-occurring IgM constant region domain(s).

As used herein, an “IgA constant region” refers to all or a portion of the three constant region domains of immunoglobulin A (isotype A1 or A2), referred to as Cal, Cα2 and Cα3 (each approximately 110 amino acids in length), including alleles (including the three human IgA constant region alleles of A1 or A2), variants and modified forms that retain the functional activity of the naturally-occurring IgA constant region domain(s).

As used herein, the term “immunoglobulin J chain” refers to an optional accessory protein of IgM or IgA that is not essential for IgM or IgA assembly (polymerization) but that enhances IgM or IgA assembly and plays a role in mucosal secretion and complement activation. An exemplary full-length human J chain has the amino acid sequence shown in SEQ ID NO: 40.

As used herein, the terms “immunoglobulin tailpiece” or “tailpiece region” refers to a short extension region at the C-terminal end of the constant region of an immunoglobulin, e.g., IgM or IgA (including all alleles thereof). The IgM and IgA tailpieces are typically about 18 amino acid regions at the carboxy terminus, which contain a cysteine that is involved in multimerization of IgM or IgA. Exemplary human IgM and IgA tailpieces have the amino acid sequences shown in SEQ ID NOs: 41 and 42, respectively.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the terms “linked,” “conjugated,” “fused,” or “fusion,” are used interchangeably when referring to the joining together of two more elements or components or domains, by whatever means including recombinant or chemical means.

As used herein, the term “linker sequence” refers to a nucleotide sequence, and corresponding encoded amino acid sequence, or “linker” that serves to link or separate two polypeptides, such as two polypeptide domains of a fusion protein. For example, an intervening linker sequence can serve to provide flexibility and/or additional space between the two polypeptides that flank the linker. A linker sequence also can contain, for example, a protease cleavage site or a ribosomal skipping sequence.

The term “major histocompatibility complex” or “MHC” refers to a genomic locus containing a group of genes that encode the polymorphic cell-membrane-bound glycoproteins known as MHC class I and class II molecules, which regulate the immune response by presenting peptides of fragmented proteins to CD8⁺ (e.g., cytotoxic) and CD4⁺ (e.g., helper) T lymphocytes, respectively. In humans, this group of genes is also called “human leukocyte antigen” or “HLA.” A HLA can be selected from multiple serotypes (e.g., HLA-A*02, HLA-DPA1*02) which each may include multiple alleles (e.g., HLA-A*02:01, HLA-DPA1*02:02). Nucleotide sequences and a gene map of human MHC are publicly available (e.g., The MHC sequencing consortium, (1999) NATURE, 401:921-923).

The terms “major histocompatibility complex” and “MHC” also refer to the polymorphic glycoproteins encoded by the MHC class I or class II genes, where appropriate in the context, and proteins comprising variants thereof that bind T cell epitopes (e.g., class I or class II epitopes). Such proteins are also referred to as “MHC molecule” or “MHC protein” herein. The terms “MHC class I” or “MHC I” are used interchangeably to refer to protein molecules comprising an α chain composed of three domains (α1, α2 and α3), and a second, invariant β2-microglobulin. The α3 domain is linked to the transmembrane domain, anchoring the MHC class I molecule to the cell membrane. Antigen-derived peptide epitopes, which are located in the peptide-binding groove, in the central region of the α1/α2 heterodimer. MHC Class I molecules such as HLA-A are part of a process that presents short polypeptides to the immune system. These polypeptides are typically 8-11 amino acids in length and originate from proteins being expressed by the cell, which can be endogenous proteins or exogenous proteins (e.g., viral or bacterial proteins, vaccine proteins). MHC class I molecules present antigen to CD8+ cytotoxic T cells. The terms “MHC class II” and “MHC II” are used interchangeably to refer to protein molecules containing an α chain with two domains (α1 and α2) and a β chain with two domains (β1 and β2). The peptide-binding groove is formed by the α1/β1 heterodimer. MHC class II molecules present antigen to specific CD4+ T cells. Antigens delivered endogenously to APCs are processed primarily for association with MHC class I. Antigens delivered exogenously to APCs are processed primarily for association with MHC class II. As used herein, MHC proteins (MHC Class I or Class II proteins) also includes MHC variants which contain amino acid substitutions, deletions or insertions and yet which still bind MHC peptide epitopes (MHC Class I or MHC Class II peptide epitopes). The term “MHC,” “MHC molecule,” or “MHC protein” also includes an extracellular fragment of a full-length MHC protein that retains the ability to bind the cognate epitope, for example, a soluble MHC. As used herein, the term “soluble MHC” refers to an extracellular fragment of a MHC comprising corresponding α1 and α2 domains that bind a class I T cell epitope or corresponding α1 and β1 domains that bind a class II T cell epitope, where the α1 and α2 domains or the α1 and β1 domains are derived from a naturally occurring MHC or a variant thereof.

The term “MHC protein” also includes MHC proteins of non-human species of vertebrates. MHC proteins of non-human species of vertebrates play a role in the examination and healing of diseases of these species of vertebrates, for example, in veterinary medicine and in animal tests in which human diseases are examined on an animal model, for example, experimental autoimmune encephalomyelitis (EAE) in mice (Mus musculus), which is an animal model of the human disease multiple sclerosis. Non-human species of vertebrates are, for example, and more specifically mice (Mus musculus), rats (Rattus norvegicus), cows (Bos taurus), horses (Equus equus) and green monkeys (Macaca mulatta). MHC proteins of mice are, for example, referred to as H-2-proteins, wherein the MHC class I proteins are encoded by the gene loci H2K, H2L, and H2D and the MHC class II proteins are encoded by the gene loci H2I.

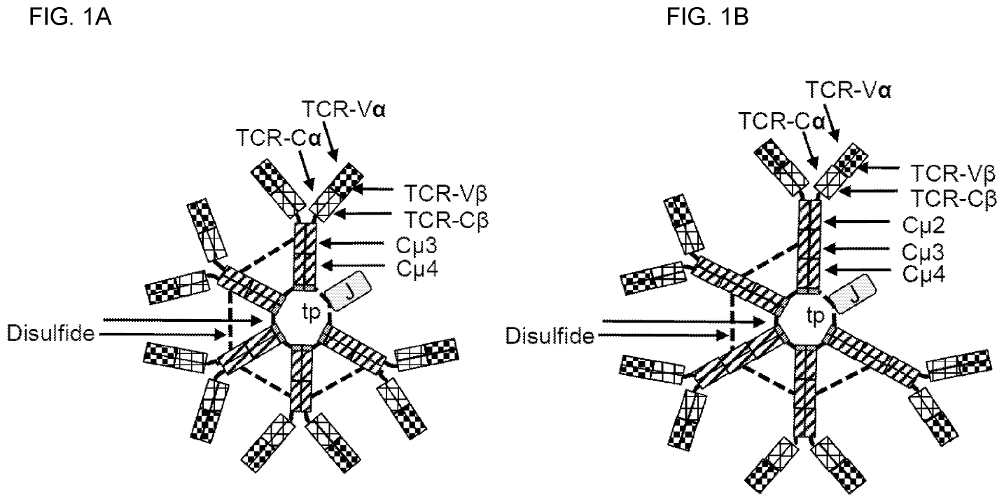

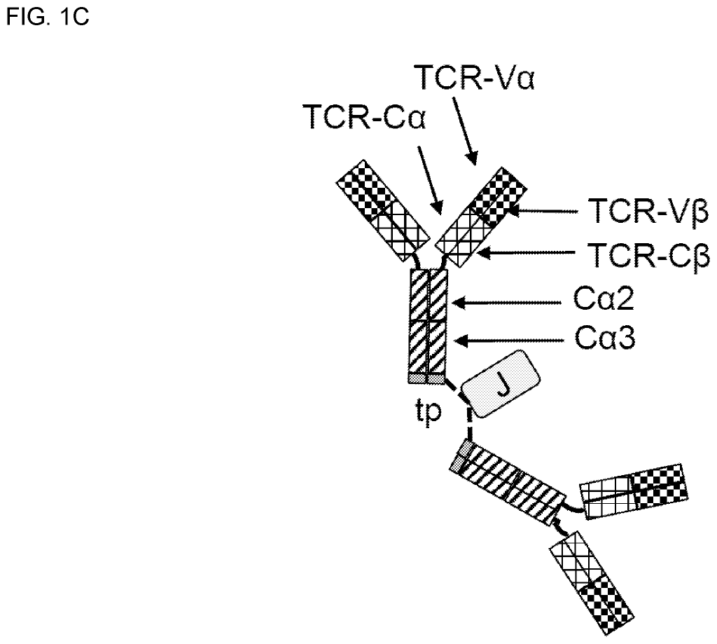

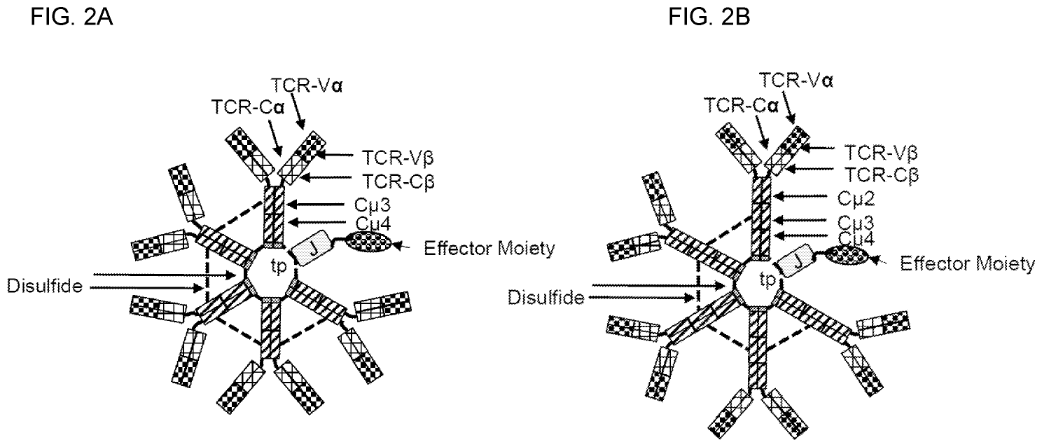

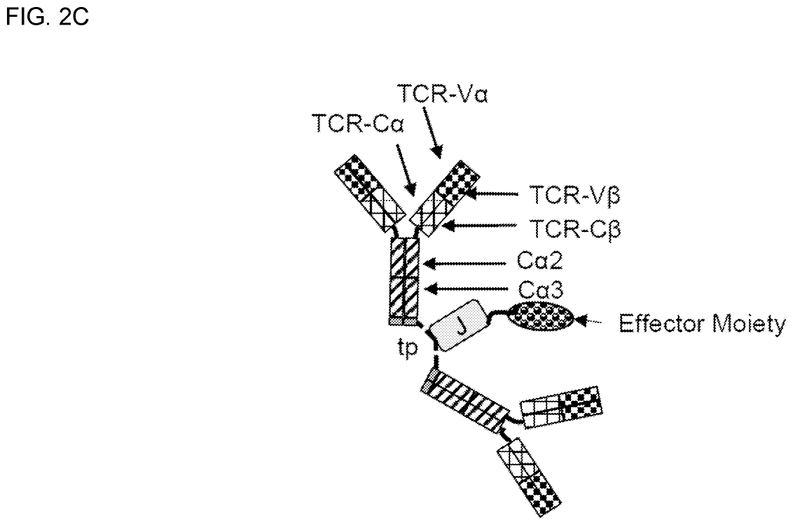

As used herein, the term “multimer” refers to a plurality (two or more) of units, e.g., a plurality of units comprising a TCR moiety-multimerization moiety fusion. The multimerization moiety is comprised of an immunoglobulin heavy chain constant region, which dimerizes to form the immunoglobulin Fc domain. As used herein, a “multimer” can be a dimer, a trimer, a tetramer, a pentamer, or a hexamer, with respect to the immunoglobulin Fc domain of the TCR multimer (e.g., corresponding to the Ig Fc region within the TCR multimer). However, since the Fc domain is a dimer and presents two TCR moieties, each unit of the multimer is bivalent (i.e., has two peptide-binding regions) and there will be 2× the number of peptide-binding sites as there are Ig subunits in each TCR multimer. Thus, for example, a “hexamer” TCR multimer comprises twelve peptide-binding sites provided by the TCR moiety and a “pentamer” TCR multimer comprises ten peptide-binding sites provided by the TCR moiety. This is illustrated, for example, in FIGS. 1A and 1B and FIGS. 2A and 2B, which each show pentamer TCR multimers (i.e., five Ig subunits) having ten peptide-binding sites provided by the TCR moiety or FIGS. 1C, and 2C, which each show exemplary IgA dimer TCR multimers.

As used herein, the terms “operatively linked” and “operably linked” are used interchangeably to describe configurations between sequences within an expression construct that allow for particular operations to be carried out. For example, when a regulatory sequence is “operatively linked” to a coding sequence within an expression construct, the regulatory sequence operates to regulate the expression of the coding sequence. Similarly, when a cleavage sequence (site) is “operatively linked” to a peptide sequence within an expression construct, cleavage at the cleavage sequence operates to cleave the peptide sequence away from the rest of the polypeptide encoded by the expression construct. Similarly, when two polypeptides within a fusion protein are “operatively linked”, the sequences encoding the two polypeptides are linked in-frame in an expression construct such that transcription and translation of the construct leads to a contiguous fusion protein comprised of the two polypeptides.

As used herein, “percent identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Similarly, percent “identity” between a nucleic acid sequence and a reference sequence is defined as the percentage of nucleotides in the nucleic acid sequence that are identical to the nucleotides in the reference sequence, after aligning the sequences and introducing gaps, if necessary, to achieve the maximum percent sequence identity. Alignment for purposes of determining percent sequence identity (e.g., amino acid sequence identity or nucleic acid sequence identity) can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

As used herein, “pharmaceutical composition” or “pharmaceutical formulation” refers to the combination of an active agent with a carrier, inert or active, making a composition especially suitable for diagnostic or therapeutic use in vivo or ex vivo.

As used herein, the phrases “pharmaceutically acceptable” and “pharmacologically acceptable,” refer to compounds, molecular entities, compositions, materials, and/or dosage forms that do not produce an adverse, allergic or other untoward reaction when administered to an animal, or a human, as appropriate. For human administration, preparations should meet sterility, pyrogenicity, and general safety and purity standards as required by FDA Office of Biologics standards or its international equivalents. “Pharmaceutically acceptable” and “pharmacologically acceptable” can mean approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable excipient” refers to a substance that aids administration of an active agent to and/or absorption by a subject and can be included in the compositions of the present disclosure without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. For examples of excipients, see, e.g., REMINGTON'S PHARMACEUTICAL SCIENCES, 18th Edition (Easton, Pennsylvania: Mack Publishing Company, 1990.

As used herein the terms “polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The terms “isolated protein” and “isolated polypeptide” are used interchangeably to refer to a protein (e.g., a soluble, multimeric protein) which has been separated or purified from other components (e.g., proteins, cellular material) and/or chemicals. Typically, a polypeptide is purified when it constitutes at least 60 (e.g., at least 65, 70, 75, 80, 85, 90, 92, 95, 97, or 99) % by weight of the total protein in the sample.

As used herein, “subject” and “patient” are used interchangeably and refer to an organism to be treated by the methods and compositions provided herein. Such organisms are preferably a mammal (e.g., human, mouse, rat, guinea pig, dog, cat, horse, cow, pig, or non-human primate, such as a monkey, chimpanzee, baboon, and rhesus), and more preferably, a human.

The term “substantial identity” indicates that two amino acid sequences, when optimally aligned and compared, are identical, with appropriate insertions or deletions, in at least about 90% of the amino acids and usually at least about 95% of the amino acids. In various embodiments, the two sequences may be at least 90%, 91%, 92%, 93%, 94%, 95%, 96%, 97%, 98%, 99% or 99.5% identical at the amino acid sequence level.

As used herein, the term “tag” refers to an additional molecular component that is affixed to a TCR multimer to thereby label it, for example for detection and/or purification purposes. A “tag” encompasses polypeptide or peptide molecules that can serve as detectable labels or targets used in purification e.g., by affinity chromatography (e.g., 6×His, Flag, V5 tags, described further herein). A “tag” also can be an oligonucleotide component, generally DNA, that provides a means of addressing a target molecule (e.g., a TCR multimer) to which it is joined (described further herein).

As used herein, the terms “T cell receptor” and “TCR” refer to a surface protein (e.g., a heterodimeric protein) of a T cell that allows the T cell to recognize an antigen and/or an epitope thereof, typically presented by a major histocompatibility complex (MHC), or a fragment of such surface protein comprising at least its variable domains. Typically, TCRs are heterodimers comprising two different protein chains. In many T cells, the TCR comprises an alpha (α) chain and a beta (β) chain. Each chain, in its native form, typically comprises two extracellular domains, a variable (V) domain and a constant (C) domain, the latter of which is membrane-proximal. The variable domain of α-chain (Vα) and the variable domain of β-chain (Vβ) each comprise three hypervariable regions that are also referred to as the complementarity determining regions (CDRs) such as CDR1, CDR2, and CDR3. The CDRs, in particular CDR3, are primarily responsible for contacting epitopes and thus define the specificity of the TCR, although CDR1 of the α-chain can interact with the N-terminal part of the antigen, and CDR1 of the β-chain interacts with the C-terminal part of the antigen. All numbering of the amino acid sequences and designation of protein loops and sheets of TCRs is according to the IMGT numbering scheme (the international ImMunoGeneTics information system; Lefranc et al. (2003) DEV. COMP. IMMUNOL., 27:55 77; Lefranc et al. (2005) DEV. COMP. IMMUNOL., 29:185-203). The terms “T cell receptor” and “TCR” also include an “engineered T cell receptor” or “engineered TCR,” such as a recombinantly modified protein comprising a fragment of a naturally occurring TCR that bind a T cell epitope-MHC complex, or a variant of such fragment. For example, an engineered TCR may contain a modified binding cassette (e.g., where one or more CDR sequences or other elements is modified, for example, by introducing corresponding sequences from a different TCR). For example, the α and/or β chain CDR3 sequences of a first TCR identified herein may be introduced into a second, different TCR present in or derived from a given T cell. The TCR may also contain modification, truncation, or deletion of its constant region, hinge region, transmembrane region, and/or intracellular region. For example, at least the transmembrane region and the intracellular region can be deleted to generate a soluble TCR. According, in certain embodiments, a TCR (e.g., engineered TCR) comprises corresponding Vα and Vβ domains that bind a T cell epitope-MHC complex, where the Vα and Vβ domains are derived from a naturally occurring TCR or a variant thereof.

As used herein, the term “TCR moiety” refers to a portion of a TCR multimer of the disclosure that includes the variable domains of the TCR alpha (α)/beta (β) chains or gamma (γ)/delta (δ) chains, including alleles, variants and modifications thereof that retain the functional properties of the TCR V region, as described further herein.

As used herein, a “TCR/pMHC complex” refers to a protein complex formed by binding between a T cell receptor (TCR), or a soluble portion thereof, and a peptide-loaded MHC molecule. Accordingly, a “component of a TCR/pMHC complex” refers to one or more subunits of a TCR (e.g., Vα, Vβ, Cα, Cβ), or to one or more subunits of an MHC or pMHC class I or II molecule.

As used herein, the terms “treat”, “treating” or “treatment” include any effect, for example, lessening, reducing, modulating, ameliorating, inhibiting (such as arresting development, either in a first instance or recurrence), or eliminating, that results in the prevention or improvement of the condition, disease, disorder, and the like, or ameliorating a symptom thereof. Treating can be curing, improving, or at least partially ameliorating (including relieving by regression of a disease state) the disorder. In certain embodiments, treating is curing the disease. As used herein, “reducing” or “reduction” of a symptom or symptoms (and grammatical equivalents of this phrase) means decreasing of the severity or frequency of the symptom(s), or elimination of the symptom(s). Treating includes treatment of a disease in a subject, such as a human.

As used herein, a “variant” refers to a modified form of the protein or polypeptide in which one or more modifications, such as amino acid swaps, substitutions, deletions and/or insertions, have been made such that the amino acid sequence of the variant differs from the parental amino acid sequence from which it is derived. Thus, a “variant” is derived from a parental protein or polypeptide through introduction of one or more modifications. Typically, a “variant” retains “substantial identity” to the parental protein or polypeptide from which it is derived.

### II. TCR Multimer Components

The TCR multimers of the disclosure comprise a TCR moiety that is operatively linked to a multimerization moiety. As provided herein, a TCR moiety comprises at least the variable (V) region of a T cell receptor, either an α/β receptor or a γ/δ receptor. The multimerization moiety comprises at least one constant (C) region and a tailpiece region of IgM or IgA. In some embodiments, a TCR moiety and a multimerization moiety are fused to form a fusion protein. In some such embodiments, this multimerization moiety mediates polymerization of the fusion protein into multimers, which can be dimers, trimers, tetramers, pentamers, or hexamers. The resultant TCR multimer is a soluble multivalent molecule that can bind to peptide-MHC complexes recognized by the TCR variable region portion of the multimer. A composition comprising the TCR multimers can comprise a single type of multimer (e.g., only pentamers or only hexamers). Alternatively, a composition comprising the multimers can comprise a mixture of at least two multimers selected from the group consisting of dimers, trimers, tetramers, pentamers, or hexamers (e.g., a mixture of hexamers and pentamers or a mixture of pentamers and dimers).

Accordingly, in one aspect, the disclosure provides a TCR multimer comprising a TCR moiety where (a) the TCR moiety comprises (i) a TCR α chain variable (V) region and a TCR β chain V region; or (ii) a TCR γ chain V region and a TCR δ chain V region; and (b) the multimerization moiety comprises (i) an IgM Cμ4 constant region and an IgM tailpiece region; or (ii) an IgA Cα3 constant region and an IgA tailpiece region.

The TCR multimers optionally can also include an immunoglobulin J chain, an effector moiety, and/or a functional moiety. Non-limiting representative TCR multimer structures are illustrated schematically in FIGS. 1A-1C and FIGS. 2A-2C. The various components of the TCR multimers are provided by the present disclosure and described in further detail below.

TCR multimers as provided herein comprise one or more moieties and each moiety may be made up of one or more domain. A domain may refer to a separately folding polypeptide entity (i.e., a structural domain), or, given context, may refer to particular regions of molecules, such as, e.g., a “VH domain” or “VL domain” such as from an antibody or antibody fragment. For example, a particular moiety may be made up of one or more domains (e.g., an IgM has four Fc domains, whereas an anti-CD3 scFv molecule is a single domain). A region may also comprise one or more domains, such as, for example, a multi-domain polypeptide (e.g., the extracellular region of PD-L1, which as two domains).

### A. TCR Moiety

The TCR moiety portion of the TCR multimer comprises either TCR alpha (α) and beta (β) chain V regions or TCR gamma (γ) and delta (δ) chain V regions, and mediates the antigen-specific binding of the multimer to peptide-MHC complexes. The native TCR is a disulfide-linked membrane bound heterodimeric protein that is expressed as part of a complex with the invariant CD3 chain. Approximately 95% of human T cells express α/β TCR chains, with about 5% expressing γ/δ TCR chains. Each native TCR subunit comprises a variable (V) region and a constant (C) region, although it is the pairing of the V region subunits (α/β or γ/δ) that forms the peptide-MHC binding site. Accordingly, in certain embodiments, the TCR moiety of the multimer contains only the V regions of the TCR subunits (α/β or γ/δ). In another embodiment, the TCR moiety further includes the C regions of the TCR subunits as well. Thus, in some such embodiments, the TCR moiety comprises TCR α and β chain V regions and further comprises TCR α and β chain C regions. In another embodiment, the TCR moiety comprises TCR γ and δ chain V regions and further comprises TCR γ and δ constant (C) regions.

The multimerization moiety can be operatively linked to either TCR subunit of an α/β or γ/δ pair. In some embodiments, the TCR moiety is operatively linked to the multimerization moiety through the TCR α chain or the TCR γ chain. In another embodiment, the TCR moiety is operatively linked to the multimerization moiety through the TCR β chain or the TCR δ chain. The TCR moiety and the multimerization moiety can be separated by a linker, such as a flexible linker, a rigid linker or a semi-rigid linker, as described further herein.

In some embodiments, the two TCR subunits (α/β or γ/δ) are expressed (e.g., in a host cell) as separate soluble molecules that self-assemble into α/β or γ/δ pairs. In another embodiment, the TCR subunit V regions are operatively linked into a single chain TCR construct.

Numerous TCR α/β and γ/δ sequences are known in the art can be used in the TCR multimers of the disclosure. A non-limiting example of a suitable TCR is an α/β TCR called D046C1. The D046C1 TCR has an α chain having the amino acid sequence shown in SEQ ID NO: 1, with the variable region shown in SEQ ID NO: 2 and the constant region shown in SEQ ID NO: 3. The D046C1 TCR has a β chain having the amino acid sequence shown in SEQ ID NO: 4, with the variable region shown in SEQ ID NO: 5 and the constant region shown in SEQ ID NO: 6. The D046C1 TCR is specific for a cytomegalovirus (CMV) pp65 epitope having the sequence NLVPMVATV (SEQ ID NO: 7), restricted to MHC Class I HLA-A2*01, which has the full-length and extracellular region sequences shown in SEQ ID NOs: 8 and 9, respectively. To form the peptide-MHC complex recognized by the TCR, the HLA-A chain pairs with β-2 microglobulin, and this heterodimer binds the epitope in its peptide-binding groove. The full-length human β-2 microglobulin amino acid sequence shown in SEQ ID NO: 10.

As used herein, a “TCR moiety” is intended to encompass variants, mutants and otherwise modified forms of the TCR α/β or γ/δ pairs that still retain the antigenic binding specificity of the parent TCR from which the modified form is derived. Such variants have been described in the art (see e.g., Wagner et al. (2019) J. BIOL. CHEM., 294:5790-5804). Such variants include modifications within a TCR sequence that eliminate N-linked glycosylation motifs, modifications such as introducing additional disulfide bonds to improve stability (e.g., by substituting non-cysteine residues with cysteine residues), or elimination of cysteine residues by substitution with another amino acid, and/or elimination of free cysteines (e.g., Cβ) (e.g., as described in Wagner et al. (2019) J. BIOL. CHEM., 294:5790-5804). In some aspects, variants include modifications within the TCR constant domains that improve the stability of the TCR. Such variants have been described in the art (see e.g., Froning, et al. (2020) Nat. Commun., Vol. 11, Article number: 2330).

For example, in some embodiments, TCR variants can be prepared to introduce modifications into α and/or β chain sequences. For instance, modifications may be introduced into the D046C1 TCR α chain sequence and/or β chain sequence to mutate one or more asparagine (N) residues that are the site of N-linked glycosylation and/or to alter disulfide-bonds by introducing or eliminating cysteine residues. To give but one example, mutations are introduced into the α and/or β chains of the TCR called D046C1. The D046C1 TCR has an α chain having the amino acid sequence shown in SEQ ID NO: 1, with the variable region shown in SEQ ID NO: 2 and the constant region shown in SEQ ID NO: 3. The D046C1 TCR has a β chain having the amino acid sequence shown in SEQ ID NO: 4, with the variable region shown in SEQ ID NO: 5 and the constant region shown in SEQ ID NO: 6. The D046C1 TCR is specific for a cytomegalovirus (CMV) pp65 epitope having the sequence NLVPMVATV (SEQ ID NO: 7), restricted to MHC Class I HLA-A2*01, which has the full-length and extracellular region sequences shown in SEQ ID NOs: 8 and 9, respectively. The full-length human β-2 microglobulin that pairs with the HLA-A molecule has the amino acid sequence shown in SEQ ID NO: 10.

Mutations can be made in the D046C1 TCR α and/or β chains by standard recombinant DNA technology. Two representative mutants each of the α chain and β chain are shown below, compared to the wild-type sequences. Different TCR variable germline regions have differing numbers of consensus N-linked glycosylation sites, so the indicated mutations are representative only. Moreover, additional modifications can be made to introduce disulfide bonds. These modified TCR multimers are expressed and purified as described in Example 1.

In all sequences shown below, the variable region is underlined and representative amino acids positions that can be selected for mutation, alone or in combination, are bolded.

### TCR D046C1 A Chain Mutations:

TCR D046C1-α chain wild-type sequence
(SEQ ID NO: 1)
KNEVEQSPQNLTAQEGEFITINCSYSVGISALHWLQQHPGGGIVS
LFMLSSGKKKHGRLIATINIQEKHSSLHITASHPRDSAVYICAAD
PSDSWGKLQFGAGTQVVVTPDIQNPDPAVYQLRDSKSSDKSVCLF
TDFDSQTNVSQSKDSDVYITDKTVLDMRSMDFKSNSAVAWSNKSD
FACANAFNNSIIPEDTFFPSP
TCR D046C1-α chain Mutant #1
(mutations introduced at the following residues,
using IMGT numbering: Vα: N22Q, Cα: N38Q,
Cα: T84C, Cα: N90Q)
(SEQ ID NO: 11)
KNEVEQSPQNLTAQEGEFITIQCSYSVGISALHWLQQHPGGGIVS
LFMLSSGKKKHGRLIATINIQEKHSSLHITASHPRDSAVYICAAD
PSDSWGKLQFGAGTQVVVTPDIQNPDPAVYQLRDSKSSDKSVCLF
TDFDSQTQVSQSKDSDVYITDKCVLDMRSMDFKSNSAVAWSQKSD
FACANAFNNSIIPEDTFFPSP
TCR D046C1-α chain Mutant #2
(mutations introduced at the following residues,
using IMGT numbering: Vα: N22Q, Cα: T84C,
Cα: N90Q, Cα: N109Q)
(SEQ ID NO: 12)
KNEVEQSPQNLTAQEGEFITIQCSYSVGISALHWLQQHPGGGIVS
LFMLSSGKKKHGRLIATINIQEKHSSLHITASHPRDSAVYICAAD
PSDSWGKLQFGAGTQVVVTPDIQNPDPAVYQLRDSKSSDKSVCLF
TDFDSQTNVSQSKDSDVYITDKCVLDMRSMDFKSNSAVAWSQKSD
FACANAFQNSIIPEDTFFPSP

### TCR D046C1 B Chain Mutations:

TCR D046C1-β chain wild-type sequence
(SEQ ID NO: 4)
EPEVTQTPSHQVTQMGQEVILRCVPISNHLYFYWYRQILGQKVEF
LVSFYNNEISEKSEIFDDQFSVERPDGSNFTLKIRSTKLEDSAMY
FCASSESRILTYNEQFFGPGTRLTVLEDLNKVFPPEVAVFEPSEA
EISHTQKATLVCLATGFFPDHVELSWWVNGKEVHSGVSTDPQPLK
EQPALNDSRYCLSSRLRVSATFWQNPRNHFRCQVQFYGLSENDEW
TQDRAKPVTQIVSAEAWGRADC
TCR D046C1-β chain Mutant #1
(mutations introduced at the following residues,
using IMGT numbering: Vβ: N86Q, Cβ: S79C,
Cβ: N85.6Q)
(SEQ ID NO: 13)
EPEVTQTPSHQVTQMGQEVILRCVPISNHLYFYWYRQILGQKVEF
LVSFYNNEISEKSEIFDDQFSVERPDGSQFTLKIRSTKLEDSAMY
FCASSESRILTYNEQFFGPGTRLTVLEDLNKVFPPEVAVFEPSEA
EISHTQKATLVCLATGFFPDHVELSWWVNGKEVHSGVCTDPQPLK
EQPALQDSRYCLSSRLRVSATFWQNPRNHFRCQVQFYGLSENDEW
TQDRAKPVTQIVSAEAWGRADC
TCR D046C1-β chain Mutant #2
(mutations introduced at the following residues,
using IMGT numbering: Vβ: N86Q, Cβ: S79C,
Cβ: N85.6Q, Cβ: C85.1A)
(SEQ ID NO: 14)
EPEVTQTPSHQVTQMGQEVILRCVPISNHLYFYWYRQILGQKVEF
LVSFYNNEISEKSEIFDDQFSVERPDGSQFTLKIRSTKLEDSAMY
FCASSESRILTYNEQFFGPGTRLTVLEDLNKVFPPEVAVFEPSEA
EISHTQKATLVCLATGFFPDHVELSWWVNGKEVHSGVCTDPQPLK
EQPALQDSRYALSSRLRVSATFWQNPRNHFRCQVQFYGLSENDEW
TQDRAKPVTQIVSAEAWGRADC

Non-limiting examples of residues within a TCR α chain variable or constant region sequence that can be selected for modification include (using IMGT numbering): Vα:N22, Cα:N38, Cα:T84, Cα:N90 and Cα:N109. A modified α chain can have substitutions at, e.g., one, two, three, four or all five of these residues. SEQ ID NO: 11 shows the amino acid sequence of a modified D046C1 TCR α chain having the following substitutions (using IMGT numbering): Vα:N22Q, Cα:N38Q, Cα:T84C and Cα:N90Q. SEQ ID NO: 12 shows the amino acid sequence of a modified D046C1 TCR α chain having the following substitutions (using IMGT numbering): Vα:N22Q, Cα:T84C, Cα:N90Q, Cα:N109Q.

Non-limiting examples of residues within a TCR β chain variable or constant region sequence that can be selected for modification include (using IMGT numbering): Vβ:N86, Cβ:S79, Cβ:N85.6 and Cβ:C85.1A. A modified β chain can have substitutions at, e.g., one, two, three or all four of these residues. SEQ ID NO: 13 shows the amino acid sequence of a modified D046C1 TCR β chain having the following substitutions (using IMGT numbering): Vβ:N86Q, Cβ:S79C and Cβ:N85.6Q. SEQ ID NO: 14 shows the amino acid sequence of a modified D046C1 TCR β chain having the following substitutions (using IMGT numbering): Vβ:N86Q, Cβ:S79C, Cβ:N85.6Q and Cβ:C85.1A.

### B. Multimerization Moiety

The multimerization moiety portion of the TCR multimer comprises at least one region (domain) from IgM or IgA sufficient such that multimerization occurs. In various embodiments, at least the most C-terminal constant region of either an IgM or IgA molecule (Cμ4 or Cα3, respectively) is used in the multimerization moiety, as well as a tailpiece region from either IgM or IgA. A constant region or a plurality of constant regions from IgM can be paired with a tailpiece region from either IgM or IgA. Likewise, a constant region(s) from IgA can be paired with a tailpiece region from either IgM or IgA. In some embodiments, the multimerization moiety can include one, two, three or four C regions from IgM or IgA (as described further below). In some embodiments, the multimerization moiety uses only the IgM or IgA tailpiece region without any constant regions.

The native IgM constant region contains four distinct constant region domains, Cμ1, Cμ2, Cμ3 and Cμ4, each approximately 110 amino acids in length. Human IgM has four alleles and the constant region sequences from any of these four alleles can be used in the TCR multimers. The constant region also includes an 18 amino acid “tailpiece”, the amino acid sequence of which in humans is shown in SEQ ID NO: 41. The predominant native form of human IgM is a pentamer, although hexamers can also form. However, hexameric IgM does not contain a J chain (i.e., the presence of a J chain prevents hexamer formation), whereas pentameric IgM may or may not include a J chain. The Cμ2 and Cμ3 domains and the tailpiece each include a cysteine that form a disulfide bond with another u chain. Deletion of the tailpiece has been shown to prevent the formation of polymeric IgM (Davis et al. (1989) J. IMMUNOL., 43:1352-1357).

Accordingly, in some embodiments, the multimerization moiety comprises an IgM Cμ4 constant region and an IgM or IgA tailpiece region.

In some embodiments, the multimerization moiety contains one additional IgM C region. Thus, the multimerization moiety can comprise (i) IgM Cμ3 and Cμ4 constant regions and IgM or IgA tailpiece region, (ii) IgM Cμ2 and Cμ4 constant regions and IgM or IgA tailpiece region or (iii) IgM Cμ1 and Cμ4 constant regions and IgM or IgA tailpiece region.

In some embodiments, the multimerization moiety contains two additional IgM C regions. Thus, the multimerization moiety can comprise (i) IgM Cμ2, Cμ3 and Cμ4 constant regions and IgM or IgA tailpiece region, (ii) IgM Cμ1, Cμ3 and Cμ4 constant regions and IgM or IgA tailpiece region or (iii) IgM Cμ1, Cμ2 and Cμ4 constant regions and IgM or IgA tailpiece region.

In some embodiments, the multimerization moiety contains three additional IgM C regions, namely IgM Cμ1, Cμ2, Cμ3 and Cμ4 constant regions and IgM or IgA tailpiece region.

The native IgA constant region contains three distinct constant region domains, Cα1, Cα2 and Cα3. Human IgA has two isotypes, A1 and A2, each of which have three alleles. The constant region sequences from any of these six alleles (three from A1 and three from A2) can be used in the TCR multimers. The IgA constant region also includes an 18 amino acid “tailpiece”, the amino acid sequence of which in humans is shown in SEQ ID NO: 42. The predominant native form of human IgA is a dimer, facilitated by the J chain, although it can also exist naturally as a monomer.

Accordingly, in certain embodiments, the multimerization moiety comprises an IgA Cα3 constant region and an IgA or IgM tailpiece region.

In some embodiments, the multimerization moiety contains one additional IgA C region. Thus, the multimerization moiety can comprise (i) IgA Cα2 and Cα3 constant regions and IgA or IgM tailpiece region or (ii) IgA Cα1 and Cα3 constant regions and IgA or IgM tailpiece region.

In some embodiments, the multimerization moiety contains two additional IgA C regions, namely IgA Cα1, Cα2 and Cα3 constant regions and IgA or IgM tailpiece region.

As used herein, a “multimerization moiety” is intended to encompass variants, mutants and otherwise modified forms of the IgM or IgA constant region sequences that still retain the polymerization ability of the IgM or IgA constant region sequences from which the modified form is derived. Such variants include modifications to eliminate N-linked glycosylation motifs, modifications that substitute a non-cysteine residue with a cysteine residue to thereby improve stability by introducing additional disulfide bonds, modifications that substitute a cysteine residue with a non-cysteine residue to eliminate disulfide bonds, variants with improved half-life or other pharmacokinetic property, variants with increased or decreased effector function (e.g., to alter complement activation or neutrophil activation) or variants that enhance the manufacturability of the multimer.

Specific modifications of Ig constant regions that alter effector function have been described in the art and can be applied to the multimerization moiety of the multimer to, e.g., alter disulfide bridging, alter half-life or alter complement activation ability. For instance, modification such as substitutions at certain amino acid positions may be used to impact certain activities (e.g., complement dependent cytotoxicity, or FcuR binding). For example, as is known to those of skill in the art, mutations in residues 309-316, based on the sequential Fc numbering scheme present at Uniprot Ref. ID P01871, can be introduced into an IgM C region to minimize complement-dependent cytotoxicity (CDC) (see e.g., WO2018/187702A2) see also Sharp et al., (2019) PROC. NATL. ACAD. SCI. USA, 116:11900-11905, reporting DLPSP residues 432-436 (residues 309-313 using UniProt P01871 numbering) are critical for C1 complement protein binding). An additional example, as is known to those of skill in the art, is described as a P311G mutation which can be introduced into an IgM C region to minimize CDC (see e.g., Chen et al. (2020) MABS, 12:1818436). Exemplary IgM C region sequences with mutations to minimize complement activation include SEQ ID NOS: 78-83. Exemplary peptide TCR-IgM complement-reduced sequences are set forth in SEQ ID NOs.: 84-85.

Other modifications such as mutations at Q387R can be introduced to reduce binding to FcuR receptors (Nyambora, et al. (2020) BBA PROTEINS AND PROTEOMICS, 1868:140266). Additional constant region modifications to increase half-life and/or reduce binding to FcuR receptor are known in the art and include those described in U.S. Patent Publication 20200239572.

### C. Immunoglobulin J Chain

In certain embodiments, a TCR multimer includes an immunoglobulin J chain (also used herein interchangeably with “J chain,” “J-chain,” or “Jch”). As provided herein, a J chain contains eight cysteine residues, two of which link the Cμ chains of IgM or the Cα chains of IgA via disulfide bridges. The J chain is not essential for polymerization of either IgM or IgA and thus is not a required component of a TCR multimer for multimerization of a multimer to occur. However, the J chain can enhance multimerization of IgM or IgA and thus, for at least this reason, may be included in a TCR multimer of the present disclosure. Additionally, the J chain plays a role in complement activation. For example, IgM hexamers that lack a J chain are more effective at activating complement than IgM pentamers that include a J chain. Thus, in situations in which it is desirable for a TCR multimer to activate complement, omission of the J chain from the multimer may be desirable. Alternatively, in situations in which it is desirable to avoid or limit complement activation (e.g., to avoid damage to epithelial membranes from complement activation), inclusion of the J chain in the multimer is desirable.

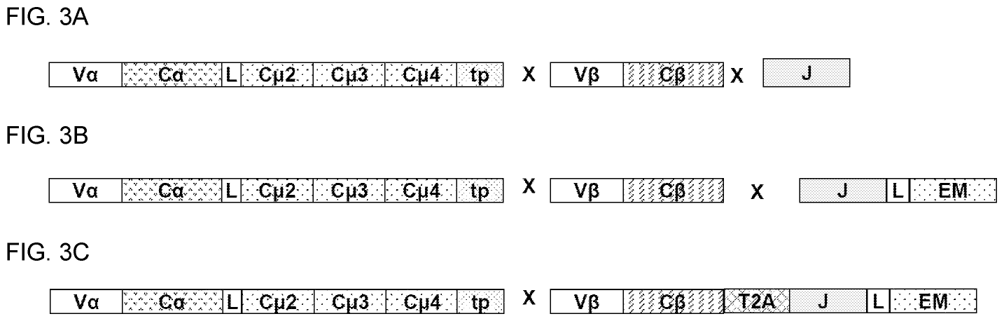

To include a J chain in the TCR multimer, an expression construct encoding the J chain can be co-transfected into a host cell along with the other TCR multimer components (see e.g., exemplary constructs in FIG. 3A and Example 1). In some embodiments, the J chain can serve as the component to which an effector moiety and/or one or more additional functional moieties can be attached. This can be accomplished recombinantly using an expression construct in which the J chain sequences are operatively linked to the effector moiety or functional moiety sequence (as illustrated schematically in FIG. 3B and FIG. 3C for anti-CD3 as the effector moiety). A linker sequence can be positioned between the J chain sequence and the effector moiety or functional moiety sequence. Suitable linker sequences are described herein. Additionally, the J chain can be expressed from a construct that also encodes one or more other components of the multimer (e.g., as shown in FIG. 3C, in which the TCR β chain sequences are encoded by the same construct as the J chain, with a ribosomal skipping sequence in between).

The full-length sequence of human J chain is shown in SEQ ID NO: 15. For instance, to give but one non-limiting example, J chain-effector moiety fusion constructs in which the anti-CD3 OKT3 scFv (SEQ ID NO: 16) is used as the effector moiety can be prepared as described in Example 3. As an additional non-limiting example, J chain-effector moiety fusion constructs in which the anti-CD3 U1v9 (D10A) scFv (SEQ ID NO: 101) is used as the effector moiety can be similarly prepared according to the protocol of Example 3. In some embodiments, the effector moiety is linked to the C-terminus of the J chain (e.g., a fusion construct as shown in SEQ ID NO: 17). In some embodiments, the effector moiety is linked to the N-terminus of the J chain (e.g., a fusion construct as shown in SEQ ID NO: 18).

### D. Effector Moiety

In certain embodiments, a TCR multimer of the disclosure includes an effector moiety. An “effector moiety” refers to a portion of the multimer that comprises one or more effector domains, e.g., one or more domains of a molecule that have or can impart biological activity, such as an immunoglobulin antigen binding site. For example, an effector moiety can “effect” or “target” specific biological responses that contribute to immune modulation, for example, by preventing or driving (i.e., trigger or advance) certain downstream activities (e.g., CD3 binding to redirect specific T cells). For avoidance of doubt, the present disclosure contemplates that the effector moiety can function in a target-specific manner via spatial localization through the TCR multimer to particular MHCs. That is, the TCR multimer(s) is/are responsible for imparting spatial localization to a target, while the effector moiety can bind to a different target (e.g., a cellular component) on or near a cell/tissue where the cognate MHC for TCR multimer is located.

In some embodiments, the effector moiety can also impart at least one additional functional property to the multimer, such as enhanced immune cell modulation or enhanced target cell killing (e.g., as compared to otherwise identical multimers not comprising the effector moiety). In some embodiments, the effector moiety can be or comprise, for example, a ligand or binding fragment thereof, a cytotoxic molecule, an antibody, an antibody fragment, peptide, or antibody-drug conjugate, an scFV, or a VHH that binds to a surface molecule of interest and/or effects one or more changes in the target cell (e.g., changes downstream activity, e.g., internalizes effector and causes cell death, etc.).

For TCR multimers that include a J chain, an effector moiety is typically appended to either the C-terminus or N-terminus of the J chain (see e.g., Example 3). For TCR multimers that lack a J chain, an effector moiety can be attached to the TCR subunit that is not fused to the multimerization domain. For example, in a multimer in which the TCR α chain (or γ chain) V region is fused to the multimerization domain, the effector moiety can be fused to the TCR β chain (or δ chain), e.g., at the C-terminal region of the C region of the β or δ chain. Alternatively, the effector moiety can be attached to the C-terminus of the multimerization moiety.

In some embodiments, the effector moiety binds to a T cell surface, thereby leading to redirection of the TCR multimer to those T cells expressing the surface antigen. Additionally, binding of the effector moiety to those T cells can modulate T cell activation and/or effector function. Non-limiting examples of suitable targeting moieties for redirection to T cells include moieties that bind a CD3 epsilon chain, a CD3 δ chain or a CD3 γ chain, as well as a pan-reactive anti-γδ-TCR moiety that can act to specifically recruit γδ-T cells.

In some embodiments, a TCR multimer of the disclosure comprises an immunoglobulin J chain linked to an effector moiety. J chain modifications are also described in, for example, U.S. Pat. No. 9,951,134. To give but one non-limiting example a J chain-effector moiety fusion can be prepared comprising an effector moiety, such as an anti-CD3 scFv, fused recombinantly to the C- or N-terminus of the J-chain such as in the J-chain fusions represented by the amino acid sequences set forth in SEQ ID NO: 17, 18, 96, 97, 99, 100, 104, and 105.

The full-length sequence of human immunoglobulin J chain is set forth in SEQ ID NO: 15. The full-length sequence of the anti-CD3 OKT3 scFv is set forth in SEQ ID NO: 16. The OKT3 scFv can be linked to either the C-terminus or the N-terminus of the J chain, to create constructs that also include a linker, e.g., ((G₄)S)₃ linker (SEQ ID NO: 19), between the J chain and scFv sequences, as well as optional Flag (DYKDDDDK) and His6 tags (HHHHHH) (SEQ ID NOs: 31 and 32, respectively) connected at the C-terminus by a linker (i.e., (GGGGS)ₙ, wherein n=1-6 (SEQ ID NO: 22)). In the constructs shown below, the J chain sequence is underlined and the OKT3 scFv sequence is bolded.

Jch-OKT3-Flag-His6
(SEQ ID NO: 17)
QEDERIVLVDNKCKCARITSRIIRSSEDPNEDIVERNIRIIVPLN
NRENISDPTSPLRTRFVYHLSDLCKKCDPTEVELDNQIVTATQSN
ICDEDSATETCYTYDRNKCYTAVVPLVYGGETKMVETALTPDACY
PDGGGGSGGGGSGGGGSQVQLQQSGAELARPGASVKMSCKASGYT
FTRYTMHWVKQRPGQGLEWIGYINPSRGYTNYNQKFKDKATLTTD
KSSSTAYMQLSSLTSEDSAVYYCARYYDDHYSLDYWGQGTTLTVS
SGGGGSGGGGSGGGGSQIVLTQSPAIMSASPGEKVTMTCSASSSV
SYMNWYQQKSGTSPKRWIYDTSKLASGVPAHFRGSGSGTSYSLTI
SGMEAEDAATYYCQQWSSNPFTFGSGTKLEIKGGGGSDYKDDDDK
HHHHHH
OKT3-Jch-Flag-His6
(SEQ ID NO: 18)
QVQLQQSGAELARPGASVKMSCKASGYTFTRYTMHWVKQRPGQGL
EWIGYINPSRGYTNYNQKFKDKATLTTDKSSSTAYMQLSSLTSED
SAVYYCARYYDDHYSLDYWGQGTTLTVSSGGGGSGGGGSGGGGSQ
IVLTQSPAIMSASPGEKVTMTCSASSSVSYMNWYQQKSGTSPKRW
IYDTSKLASGVPAHFRGSGSGTSYSLTISGMEAEDAATYYCQQWS
SNPFTFGSGTKLEIKGGGGSGGGGSGGGGSQEDERIVLVDNKCKC
ARITSRIIRSSEDPNEDIVERNIRIIVPLNNRENISDPTSPLRTR
FVYHLSDLCKKCDPTEVELDNQIVTATQSNICDEDSATETCYTYD
RNKCYTAVVPLVYGGETKMVETALTPDACYPDGGGGSDYKDDDDK
HHHHHH
Jch-OKT3
(SEQ ID NO: 99)
QEDERIVLVDNKCKCARITSRIIRSSEDPNEDIVERNIRIIVPLN
NRENISDPTSPLRTRFVYHLSDLCKKCDPTEVELDNQIVTATQSN
ICDEDSATETCYTYDRNKCYTAVVPLVYGGETKMVETALTPDACY
PDGGGGSGGGGSGGGGSQVQLQQSGAELARPGASVKMSCKASGYT
FTRYTMHWVKQRPGQGLEWIGYINPSRGYTNYNQKFKDKATLTTD
KSSSTAYMQLSSLTSEDSAVYYCARYYDDHYSLDYWGQGTTLTVS
SGGGGSGGGGSGGGGSQIVLTQSPAIMSASPGEKVTMTCSASSSV
SYMNWYQQKSGTSPKRWIYDTSKLASGVPAHFRGSGSGTSYSLTI
SGMEAEDAATYYCQQWSSNPFTFGSGTKLEIK
OKT3-Jch
(SEQ ID NO: 100)
QVQLQQSGAELARPGASVKMSCKASGYTFTRYTMHWVKQRPGQGL
EWIGYINPSRGYTNYNQKFKDKATLTTDKSSSTAYMQLSSLTSED
SAVYYCARYYDDHYSLDYWGQGTTLTVSSGGGGSGGGGSGGGGSQ
IVLTQSPAIMSASPGEKVTMTCSASSSVSYMNWYQQKSGTSPKRW
IYDTSKLASGVPAHFRGSGSGTSYSLTISGMEAEDAATYYCQQWS
SNPFTFGSGTKLEIKGGGGSGGGGSGGGGSQEDERIVLVDNKCKC
ARITSRIIRSSEDPNEDIVERNIRIIVPLNNRENISDPTSPLRTR
FVYHLSDLCKKCDPTEVELDNQIVTATQSNICDEDSATETCYTYD
RNKCYTAVVPLVYGGETKMVETALTPDACYPD

In some embodiments, the effector moiety binds to an NK cell surface antigen, thereby leading to redirection of those NK cells expressing the surface antigen to the TCR multimer, or to TCR-multimer-bound cells. Additionally, binding of the effector moiety to those NK cells can modulate NK cell activation and/or effector function. Non-limiting examples of suitable targeting moieties for redirection of NK cells include moieties that bind CD56, CD16, NKp46, CD64 or NKG2D.

In some embodiments, the effector moiety binds to a macrophage surface antigen, thereby leading to redirection of those macrophages expressing the surface antigen to the TCR multimer, or to TCR-multimer-bound cells. Additionally, binding of the effector moiety to those macrophages can modulate macrophage activation and/or effector function. A non-limiting example of a suitable effector moiety for redirection of macrophages is a moiety that binds CD14.

In some embodiments, the effector moiety binds to a neutrophil surface antigen, thereby leading to redirection of those neutrophils expressing the surface antigen to the TCR multimer, or to TCR-multimer-bound cells. Additionally, binding of the effector moiety to those neutrophils can modulate neutrophil activation and/or effector function. Non-limiting examples of suitable targeting moieties for redirection to neutrophils include moieties that bind CD16b and CD177.

In another embodiment, the effector moiety is an Fc receptor (FcR) binding molecule, thereby redirecting the TCR multimer to FcR-expressing cells, which includes a variety of immune cells (e.g., B cells, follicular dendritic cells, NK cells, macrophages, neutrophils, eosinophils). For example, the effector moiety can be an FcγR-binding moiety, which can be used e.g., to drive ADCC activity by the TCR multimer.

In another embodiment, the effector moiety is or comprises a checkpoint inhibitor, for example, an antagonist anti-PD1, anti-TIGIT, anti-CTLA4, anti-TIM3, anti-Lag3, anti-LILRB1, or anti-LILRB2 antibody or an antigen-binding fragment of any of these antibodies.

In another embodiment, the effector moiety is or comprises, for example, a PD-L1 (Domain 1 or Full ECD) or an anti-PD1 agonist antibody domain (induction of exhaustion of target T cells); an IL-10 (context-dependent immunosuppression), an HLA-G fusion (immunosuppression); an anti-LILRB1 or LILRB2 agonist (immunosuppression), or a checkpoint agonists (including agonist antibodies) to other cell surface receptors like CTLA4 agonist, Lag3 agonist, TIGIT agonist, or TIM3 agonist.

In another embodiment, the effector moiety is either a cytokine or is directed against (binds to) a cytokine. For example, the effector moiety can be a cytokine that retains its receptor binding capacity, thereby directing the multimer to cells expressing the cytokine receptor. Non-limiting examples of suitable cytokines as targeting moieties include IL-2 or a mutein thereof (e.g., an IL2 mutein that has reduced/no binding affinity for the CD25/IL2Rα receptor), IL-12, and IL-15.

In another embodiment, the effector moiety is or comprises a cell death inducing agent, for example, a tubulin inhibitor, DNA damaging agent, FasL, α-Fas agonist antibody or an antigen-binding fragment of any of these antibodies.

In another embodiment, the effector moiety is or comprises a TNFR Superfamily Protein, for example, 4-1BBL (CD137L) or a domain thereof, an α-CD137 agonist antibody, an anti-TNFR2 agonist antibody, or an antigen-binding fragment of any of these antibodies.

In addition to the foregoing, various modified J chain constructs have been described in the art that comprise a J chain fused to an effector moiety, which also are suitable for use in the TCR multimers of the disclosure. Non-limiting examples include the modified J chains described in U.S. Pat. No. 9,951,134 and U.S. Patent Publication No. 2019/0185570.

Such J chain-effector moiety constructs as provided by the present disclosure can be transfected into a host cell and coexpressed with TCR multimer construct(s), such as those described herein, to prepare multimers comprising a J chain and an effector moiety.

Non-limiting examples of heterologous functional moieties include moieties that increase the half-life, or other pharmacokinetic properties, of the multimer, cytokines (e.g., IL-2, IL-12, IL-15) and cytokine receptors (e.g., IL-2R, IL-12R, IL-15R).

### E. Additional Functional Moieties

In certain embodiments, a TCR multimer provided by the disclosure includes one or more additional heterologous functional moieties. Such a “functional moiety” as used herein refers to one or more additional regions (e.g., comprising one or more additional domains) that are incorporated into the multimer to thereby impart one or more functional properties of interest to the multimer, but like an effector moiety, does not primarily serve to direct or redirect the location of the TCR multimer. A “heterologous” functional moiety refers to the domain or region being from a molecule (e.g., protein, polypeptide) other than the TCR used in the TCR moiety and the Ig C region(s) used in the multimerization domain. Non-limiting examples of heterologous functional moieties include moieties that increase the half-life, or other pharmacokinetic properties, of the TCR multimer, cytokines (e.g., IL-2, IL-12, IL-15) and cytokine receptors (IL-2R, IL-12R, IL-15R). A heterologous functional moiety can be incorporated into a TCR multimer in a similar manner as that described above for incorporating an effector moiety.

### F. Linkers

In the TCR multimer compositions, in certain embodiments, a linker (also referred to as a spacer) is positioned between the TCR moiety and the multimerization moiety. Linkers can also be positioned between other domains or regions of various components of the TCR multimer. In some embodiments, a multimer does not comprise a linker. For example, in some embodiments, multimers provided by the present disclosure comprise TCR and IgM constant domains that do not comprise a linker in between.

The terms “linker” or “spacer” denotes a linear amino acid chain of natural and/or synthetic origin. The linkers described herein are designed to ensure that polypeptides conjugated to each other can perform their biological activity by allowing the polypeptides to fold correctly and to be presented properly. The linker may contain repetitive amino acid sequences or sequences of naturally occurring polypeptides. In some embodiments, a peptide linker has a length of from 2 to 50 amino acids. In some embodiments, a peptide linker is between 3 and 30 amino acids, between 5 to 25 amino acids, between 5 to 20 amino acids, or between 10 and 20 amino acids.

In certain embodiments, the sequence of the linker serves primarily to provide additional space (distance) between domains/regions of a fusion protein. In other embodiments, the sequence of the linker imparts one or more additional functionalities, such as a protease cleavage site to allow cleavage of a fusion protein containing the linker or a ribosomal skipping site to allow for translation of two distinct polypeptides from a fusion mRNA, as described further herein.

In certain embodiments, the linker is a flexible linker, e.g., composed of a glycine-serine-rich sequence, such as the linker shown in SEQ ID NO: 19. In another embodiment, the spacer linker is a rigid linker, e.g., composed of a proline-rich sequence, such as the linker shown in SEQ ID NO: 20. In yet another embodiment, the spacer linker is a flexible-rigid linker, comprising both a flexible region (e.g., a glycine-serine-rich sequence) and a rigid region (e.g., a proline-rich sequence), such as the linker shown in SEQ ID NO: 21.

In various other embodiments, the peptide linker is rich in glycine, glutamine, and/or serine residues. These residues are arranged e.g., in small repetitive units of up to five amino acids. This small repetitive unit may be repeated for one to five times. At the amino- and/or carboxy-terminal ends of the multimeric unit up to six additional arbitrary, naturally occurring amino acids may be added. Other synthetic peptidic linkers are composed of a single amino acid, which is repeated between 10 to 20 times and may comprise at the amino- and/or carboxy-terminal end up to six additional arbitrary, naturally occurring amino acids. All peptidic linkers can be encoded by a nucleic acid molecule and therefore can be recombinantly expressed. As the linkers are themselves peptides, the polypeptide connected by the linker are connected to the linker via a peptide bond that is formed between two amino acids.

Suitable peptide linkers are well known in the art, and are disclosed in, e.g., U.S. Patent Publication No. 2010/0210511, U.S. Patent Publication No. 2010/0179094, and U.S. Patent Publication No. 2012/0094909. Other linkers are provided, for example, in U.S. Pat. No. 5,525,491; Alfthan et al. (1995) PROTEIN ENG., 8:725-731; Shan et al. (1999) J. IMMUNOL., 162:6589-6595; Newton et al. (1996) BIOCHEMISTRY, 35:545-553; Megeed et al. (2006) BIOMACROMOLECULES, 7:999-1004; and Perisic et al. (1994) STRUCTURE, 12:1217-1226.

In some embodiments, the linker is synthetic. As used herein, the term “synthetic” with respect to a linker includes peptides (or polypeptides) which comprise an amino acid sequence (which may or may not be naturally occurring) that is linked in a linear sequence of amino acids to a sequence (which may or may not be naturally occurring) to which it is not naturally linked in nature. For example, the linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). Preferably, a linker will be relatively non-immunogenic and not inhibit any non-covalent association among monomer subunits of a binding protein.

In some embodiments, the linker is a Gly-Ser polypeptide linker, i.e., a peptide that consists of glycine and serine residues. One exemplary Gly-Ser polypeptide linker comprises the amino acid sequence (Gly₄Ser)ₙ, wherein n=1-6 (SEQ ID NO: 22). In certain embodiments, n=1. In certain embodiments, n=2. In certain embodiments, n=3. In certain embodiments, n=4. In certain embodiments, n=5. In certain embodiments, n=6. Another exemplary Gly-Ser polypeptide linker comprises the sequence SSSSGSSSSGSAA (SEQ ID NO: 23). Another linker comprises only glycine, e.g., G₅ linkers (GGGGG; SEQ ID NO: 24). Another exemplary Gly-Ser polypeptide linker comprises the amino acid sequence Ser(Gly₄Ser)ₙ, wherein n=1-10 (SEQ ID NO: 25). In certain embodiments, n=1. In certain embodiments, n=2. In certain embodiments, n=3, i.e., Ser(Gly₄Ser)₃. In certain embodiments, n=4, i.e., Ser (Gly₄Ser)₄. In certain embodiments, n=5. In certain embodiments, n=6. In certain embodiments, n=7. In certain embodiments, n=8. In certain embodiments, n=9. In certain embodiments, n=10.

Other exemplary linkers include GS linkers (i.e., (GS)n), GGSG linkers (i.e., (GGSG)n), wherein n=1-5 (SEQ ID NO: 26), GSAT linkers (SEQ ID NO: 27), SEG linkers, GGS linkers (i.e., (GGSGGS)n) (SEQ ID NO: 28), wherein n is a positive integer (e.g., 1, 2, 3, 4, or 5), (Gly₄Ser)4 (GGGGSGGGGSGGGGSGGGGS; SEQ ID NO: 29) and (GS)₂AG₂SGSG₃S linkers (GSGSAGGSGSGGGS; SEQ ID NO: 30). Other suitable linkers for use in multimeric fusion proteins can be found using publicly available databases, such as the Linker Database (ibi.vu.nl/programs/linkerdbwww). The Linker Database is a database of inter-domain linkers in multi-functional enzymes which serve as potential linkers in novel multimeric fusion proteins (see, e.g., George et al. (2002) PROTEIN ENGINEERING, 15:871-9).

In some embodiments, the linker comprises an amino acid sequence selected from the group consisting of SEQ ID NOs: 19-30.

In certain embodiments, the sequence of the linker imparts a functionality to the linker beyond simply serving as a spacer. For example, in certain embodiments, the linker comprises a protease recognition (cleavage) site such that the linker can be cleaved by a protease. In some embodiments the protease is an amino-peptidase. In other embodiments, the protease is a methionine amino-peptidase. In yet other embodiments, the protease is selected from FXa, thrombin, TEV, HRV3C and furin.

In certain embodiments, the linker sequence comprises a ribosomal skipping site (sequence) such as a 2A peptide. In an expression construct, when such a ribosomal skipping site is included in a linker positioned between two polypeptide sequences, translation of the transcribed mRNA results in production of the two polypeptides separately, rather than as a fusion protein. The 2A peptide sequences share a core sequence motif of DXEXNPGP, wherein X is any amino acid (SEQ ID NO: 43). Non-limiting examples of suitable 2A peptide sequences include T2A (EGRGSLLTCGDVEENPGP; SEQ ID NO: 44), P2A (ATNFSLLKQAGDVEENPGP; SEQ ID NO: 45), E2A (QCTNYALLKLAGDVESNPGP; SEQ ID NO: 46) and F2A (VKQTLNFDLLKLAGDVESNPGP; SEQ ID NO: 47). Additionally, an optional Gly-Ser-Gly (GSG) tripeptide can be added to the N-terminal end of a 2A peptide to enhance efficiency.

### G. Labels

In certain embodiments, one or more detectable labels are attached to a TCR multimer of the disclosure. As used herein, a “detectable label” is any molecule or functional group that allows for the detection of a biological or chemical characteristic or change in a system, such as the presence of a target substance in the sample. For example, TCR multimers can be conjugated with a fluorescent label, allowing for identification of binding the TCR multimer to a target molecule or cell, for example via flow cytometry or microscopy. Cells can also be selected based on a fluorescence label through, e.g., fluorescence or magnetic activated cell sorting.

Non-limiting examples of detectable labels include protein tags (e.g., that can be detected with an anti-tag antibody), fluorophores, chromophores, electro chemiluminescent labels, bioluminescent labels, polymers, polymer particles, bead or other solid surfaces, gold or other metal particles or heavy atoms, spin labels, radioisotopes, enzyme substrates, haptens, antigens, Quantum Dots, aminohexyl, pyrene, nucleic acids or nucleic acid analogs, or proteins, such as receptors, peptide ligands or substrates, enzymes, and antibodies (including antibody fragments).

Numerous protein tags are known in the art that can be appended at the N- or C-terminus of a polypeptide or protein to facilitate detection (e.g., using an anti-tag antibody) and/or purification (e.g., using affinity chromatography). Non-limiting examples of protein tags include a Flag tag (e.g., SEQ ID NO: 31), a 6× histidine tag (His6) (e.g., SEQ ID NO: 32), a V5 tag (e.g., SEQ ID NO: 33), a strep tag (e.g., SEQ ID NO: 34), a protein C tag (e.g., SEQ ID NO: 35), a myc tag (e.g., SEQ ID NO: 36), an avitag-Myc sequence (e.g., SEQ ID NO: 37), an avitag-Myc-His6 sequence (e.g., SEQ ID NO: 38) and/or an avitag-His6-Flag sequence (e.g., SEQ ID NO: 39), and combinations thereof.

Additional tags suitable for use in the methods and compositions provided herein include affinity tags, including but not limited to enzymes, protein domains, or small polypeptides which bind with high specificity to a range of substrates, such as carbohydrates, small biomolecules, metal chelates, antibodies, etc., to allow rapid and efficient purification of proteins. Solubility tags enhance proper folding and solubility of a protein and are frequently used in tandem with affinity tags. Sequences encoding such a tag(s) can be incorporated into an expression construct of the disclosure, such as at the C-terminus or N-terminus of the multimer-encoding regions to thereby incorporate a detectable tag into the expressed polypeptide.

Examples of enzymes which may be used comprise horse radish peroxidase (HRP), alkaline phosphatase (AP), β-galactosidase (GAL), glucose-6-phosphate dehydrogenase, β-N-acetylglucosaminidase, β-glucuronidase, invertase, Xanthine Oxidase, firefly luciferase and glucose oxidase (GO). Examples of commonly used substrates for horse radish peroxidase (HRP) include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), α-naphtol pyronin (.α.-NP),o-dianisidine (OD), 5-bromo-4-chloro-3-indolylphosphate (BCIP), Nitroblue tetrazolium (NBT), 2-(p-iodophenyl)-3-p-nitrophenyl-5-phenyltetrazolium chloride (INT), tetranitro blue tetrazolium (TNBT), .delta.-bromo-chloro-S-indoxyl-β-D-galactoside/ferro-ferricyanide (BCIG/FF). Examples of commonly used substrates for Alkaline Phosphatase include Naphthol-AS-B1-phosphate/fast red TR (NABP/FR), Naphthol-AS-MX-phosphate/fast red TR (NAMP/FR), Naphthol-AS-B1-phosphate/fast red TR (NABP/FR), Naphthol-AS-MX-phosphate/fast red TR (NAMP/FR), Naphthol-AS-B1-phosphate/new fuschin (NABP/NF), bromochloroindolylphosphate/nitroblue tetrazolium (BCIP/NBT), b-Bromo-chloro-S-indolyl-β-δ-galactopyranoside (BCIG).

Examples of luminescent labels which may be used include luminol, isoluminol, acridinium esters, 1,2-dioxetanes and pyridopyridazines. Examples of electrochemiluminescent labels include ruthenium derivatives. Examples of radioactive labels which may be used include radioactive isotopes of iodide, cobalt, selenium, hydrogen, carbon, sulfur, and phosphorous.

Some “detectable labels” also include “color labels,” in which the biological change or event in the system may be assayed by the presence of a color, or a change in color. Examples of “color labels” are chromophores, fluorophores, chemiluminescent compounds, electrochemiluminescent labels, bioluminescent labels, and enzymes that catalyze a color change in a substrate.

“Fluorophores” as described herein are molecules that emit detectable electro-magnetic radiation upon excitation with electro-magnetic radiation at one or more wavelengths. A large variety of fluorophores are known in the art and are developed by chemists for use as detectable molecular labels and can be conjugated to the MHCII multimers provided herein. Examples include FLUORESCEIN™ or its derivatives, such as FLUORESCEIN®-5-isothiocyanate (FITC), 5-(and -6)-carboxyFLUORESCEIN®, 5- or 6-carboxyFLUORESCEIN®,6-(FLUORESCEIN®)-5-(and -6)-carboxamido hexanoic acid, FLUORESCEIN® isothiocyanate, rhodamine or its derivatives such as tetramethyl rhodamine and tetramethylrhodamine-5-(and -6) isothiocyanate (TRITC). Other fluorophores include: coumarin dyes such as (diethyl-amino) coumarin or 7-amino-4-methylcoumarin-3-acetic acid, succinimidyl ester (AMCA); sulforhodamine 101 sulfonyl chloride (TEXASRED® or TEXASRED® sulfonyl chloride; 5-(and -6)-carboxyrhodamine 101, succinimidyl ester, also known as 5-(and -6)-carboxy-X-rhodamine, succinimidyl ester (CXR); lissamine or lissamine derivatives such as lissamine rhodamine B sulfonyl Chloride (LisR); 5-(and-6)-carboxyFLUORESCEIN®, succinimidyl ester (CFI); FLUORESCEIN®5-isothiocyanate (FITC);7-diethylaminocoumarin-3-carboxylic acid, succinimidyl ester (DECCA); 5-(and -6)-carboxytetramethyl-rhodamine, succinimidyl ester (CTMR); 7-hydroxycoumarin-3-carboxylic acid, succinimidyl ester (HCCA); 6->FLUORESCEIN®.-5-(and -6)-carboxamidolhexanoic acid (FCHA); N-(4,4-difluoro-5,7-dimethyl-4-bora-3a,4a-diaza-3-indacenepropionic acid, succinimidyl ester; also known as 5,7-DIMETHYLBODIPY® propionic acid, succinimidyl ester (DMBP); “activated FLUORESCEIN® derivative” (FAP), available from Probes, Inc.; eosin-5-isothiocyanate (EITC); erythrosin-5-isothiocyanate (ErlTC); and CASCADE® Blue acetylazide (CBAA) (the O-acetylazide derivative of1-hydroxy-3,6,8-pyrene-trisulfonic acid). Yet other potential fluorophores useful in this disclosure include fluorescent proteins such as green fluorescent protein and its analogs or derivatives, fluorescent amino acids such as tyrosine and tryptophan and their analogs, fluorescent nucleosides, and other fluorescent molecules such as Cy2, Cy3, Cy 3.5, CY5.TM., CY5.TM.5, Cy 7, IR dyes, Dyomics dyes, phycoerythrine, Oregon green 488, pacific blue, rhodamine green, and Alexa dyes. Yet other examples of fluorescent labels include conjugates of R-phycoerythrin orallophycoerythrin, inorganic fluorescent labels such as particles based on semiconductor material like coated CdSe nanocrystallites.

In certain embodiments, a multimer of the disclosure comprises an identifier tag or label that facilitates identification of the multimer. An “identifier” as used herein refers to a readable representation of data that provides information, such as an identity, that corresponds with the identifier. For instance, in some embodiments, an identifier is or comprises an oligonucleotide barcode. Typically, an oligonucleotide barcode is a unique oligonucleotide sequence ranging from 10 to more than 50 nucleotides. The barcode has shared amplification sequences in the 3′ and 5′ ends, and a unique sequence in the middle. This sequence can be revealed by sequencing and can serve as a specific barcode for a given molecule. The use of barcode technology is well known in the art, see for example Shiroguchi et al. (2012) PROC. NATL. ACAD. SCI. USA, 109 (4): 1347-52; and Smith et al. (2010) NUCLEIC ACIDS RESEARCH, 38 (13) 11:e142. Further methods and compositions for using barcode technology include those described in U.S. Patent Publication No. 2016/0060621. Standard methods for preparing barcode oligonucleotides and conjugating them to molecules of interest are known in the art.

In some embodiments, a TCR multimer of the disclosure is prepared as a bispecific multimer, wherein the bispecific multimer presents two different TCR molecules. Such bispecific multimers can be prepared using knobs-into-holes (KIH) technology or electrostatic steering technology, which are well-established approaches in the art for creating bispecific antibodies. KIH technology involves engineering constant domains to create either a “knob” or a “hole” in each heavy chain; while electrostatic steering involves engineering salt bridges through pairs of oppositely charged amino acids to promote heterodimerization (see e.g., U.S. Pat. No. 10,351,631). The present disclosure contemplates that KIH technology or electrostatic steering are useful for making various multimers as provided herein. For example, in some embodiments, KIH technology can be used to introduce a knob into the Cμ4 of one chain of the Ig-derived multimerization domain and a hole into the Cμ4 of the other chain to create a bispecific TCR multimer presenting two different TCR α/β pairs. In some embodiments, electrostatic steering may be used, wherein amino acid changes introducing salt-bridges may be introduced into at least one of a Cμ2, Cμ3 or Cμ4 domain of an IgM to promote heterodimerization. Such KIH technology can also be used, for example, to introduce a knob into a Cα3 of one chain of an IgA multimerization domain and a hole into a Cα3 of the other chain to create a bispecific TCR multimer presenting two different TCR α/β pairs (see e.g., U.S. Pat. No. 10,822,399).

In some embodiments, KIH technology or electrostatic steering technology may be used to make a TCR IgM multimer that includes a TCRα-IgM (“knob”) and a TCRβ-IgM (“hole”). Such a strategy would create a pentavalent TCR-IgM having five TCR moieties. Without being limited by theory, the present disclosure contemplates that, in some embodiments, KIH technology combined with an IgM could allow different TCRs to be put onto the same IgM, such that one is a α/β and the other a γ/δ.

### III. Preparation of TCR Multimers

TCR multimers of the disclosure can be prepared by methods established in the art. In some embodiments, a TCR multimer is prepared using recombinant DNA technology, as described further below. In other embodiments, a TCR multimer is prepared using chemical conjugation, various approaches for which are described further below.

### A. Recombinant Expression Strategies

As provided herein, certain exemplary recombinant expression strategies may be employed in design and manufacture of a multimer. In some embodiments, a TCR multimer composition of the disclosure is prepared by standard recombinant DNA techniques using one or more nucleic acid constructs that encode polypeptides that when expressed in a host cell self-assemble into a TCR multimer in which a TCR moiety is operatively linked to a multimerization moiety (typically with linker sequences positioned between the sequences encoding the TCR moiety and the multimerization moiety. Non-limiting representative nucleic acid constructs for expressing a TCR multimer composition are shown schematically in FIGS. 3A-3C. Use of such expression constructs for recombinantly preparing a TCR multimer are described in detail in Example 1.

General techniques for nucleic acid manipulation are well established in the art, such as described in, for example, Sambrook et al. (1989) MOLECULAR CLONING: A LABORATORY MANUAL, 2nd Edition, Vols. 1-3, Cold Spring Harbor Laboratory Press, or Ausubel et al. (1987) CURRENT PROTOCOLS IN MOLECULAR BIOLOGY, Green Publishing and Wiley-Interscience, New York and periodic updates, herein incorporated by reference. Generally, the DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding site, and sequences that control the termination of transcription and translation. The ability to replicate in a host, usually conferred by an origin of replication, and a selection gene to facilitate recognition of transformants is additionally incorporated.

In some embodiments, the nucleic acid construct is designed to be suitable for in vitro transcription/translation (IVTT). In some embodiments, the nucleic acid is designed to be suitable for recombinant expression in a host cell. Appropriate cloning and expression vectors for use with bacterial, fungal, yeast, and mammalian cellular hosts can be found in CLONING VECTORS: A LABORATORY MANUAL, (Elsevier, New York (1985)), the relevant disclosure of which is hereby incorporated by reference.

In some embodiments, the multimer composition is synthesized utilizing an in vitro transcription/translation (IVTT) system that can both transcribe, for example, a DNA construct into RNA, and then translate the RNA into a protein. IVTT can allow for protein production in a cell-free environment directly from a DNA or RNA template. An IVTT method used herein can be performed using, for example, a PCR product, a linear DNA plasmid, a circular DNA plasmid, or an mRNA template with a ribosome-binding site (RBS) sequence. After the appropriate template has been isolated, transcription components can be added to the template including, for example, ribonucleotide triphosphates, and RNA polymerase. After transcription has been completed, translation components can be added, which can be found in, for example, rabbit reticulocyte lysate, or wheat germ extract. In some methods, the transcription and translation can occur during a single step, in which purified translation components found in, for example, rabbit reticulocyte lysate or wheat germ extract are added at the same time as adding the transcription components to the nucleic acid template.

In certain embodiments, the nucleic acid sequence is incorporated into a vector, such as a plasmid vector, a viral vector or a non-viral vector. The vector is selected to be suitable for use in the intended host cell (i.e., the vector incudes all necessary transcriptional regulatory elements to allow for expression of the encoded multimer composition in the host cell). Suitable vectors, including transcriptional regulatory elements for use in various host cells, including mammalian host cells, are well established in the art.

As appreciated by those skilled in the art, because of third base degeneracy, almost every amino acid can be represented by more than one triplet codon in a coding nucleotide sequence. In addition, minor base pair changes may result in a conservative substitution in the amino acid sequence encoded but are not expected to substantially alter the biological activity of the gene product. Therefore, a nucleic acid sequence encoding a protein described herein may be modified slightly in sequence and yet still encode its respective gene product.

Nucleic acids encoding any of the various proteins or polypeptides described herein may be synthesized chemically or prepared through standard recombinant DNA techniques. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al. (2003) PROC. NATL. ACAD. SCI. USA, 100 (2): 438-442; Sinclair et al. (2002) PROTEIN EXPR. PURIF., 26 (I): 96-105; Connell, N. D. (2001) CURR. OPIN. BIOTECHNOL., 12 (5): 446-449; Makrides et al. (1996) MICROBIOL. REV., 60 (3): 512-538; and Sharp et al. (1991) YEAST, 7 (7): 657-678.

In some embodiments, the vector is designed for expression in a prokaryotic host cell (e.g. E. coli). In some embodiments, the vector is designed for expression in a eukaryotic host cell (e.g., yeast). In some embodiments, the vector is designed for expression in a mammalian host cell. In some such embodiments, the mammalian host cells are human host cells. In some embodiments, the human host cells are human embryonic kidney (HEK) cells. In some embodiments, the HEK cells are 293 cells or are a 293-derived HEK strain. Such HEK cells are commercially available in the art, a non-limiting example of which is the Expi293F™ cell line (Fisher ThermoScientific). In yet another embodiment, the mammalian host cell is a CHO cell line.

When mammalian host cells are used, typically the signal sequence used in the expression construct is derived from a mammalian protein. A signal sequence used to express a secreted protein can be a homologous signal sequence (derived from the same protein being expressed) or a heterologous signal sequence (derived from a different protein than the one being expressed). In certain embodiments, a heterologous signal sequence is from an Ig supergroup member. In some embodiments, the signal sequence is an immunoglobulin chain signal sequence. In other embodiments, the signal sequence is an Ig Kappa chain V-III region CLL signal peptide, e.g., having the sequence MEAPAQLLFLLLLWLPDTTG (SEQ ID NO: 48). Other suitable signal sequences include a human CD4 signal peptide, e.g., having the sequence MNRGVPFRHLLLVLQLALLPAAT (SEQ ID NO: 49), a mouse Ig kappa chain V-III region signal peptide, e.g., having the sequence METDTLLLWVLLLWVPGSTG (SEQ ID NO: 50), a mouse H-2Kb signal peptide, e.g., having the sequence MVPCTLLLLLAAALAPTQTRA (SEQ ID NO: 51), a human serum albumin signal peptide, e.g., having the sequence MKWVTFISLLFLFSSAYS (SEQ ID NO: 52), a human IL-2 signal peptide, e.g., having the sequence MYRMQLLSCIALSLALVINS (SEQ ID NO: 53), a human HLA-A*02:01 signal peptide, e.g., having the sequence MAVMAPRTLLLLLSGALALTQTWA (SEQ ID NO: 54) and a human β2m signal peptide, e.g., having the sequence MSRSVALAVLALLSLSGLEA (SEQ ID NO: 55).

Furthermore, the transcriptional regulatory sequences used in the vector are selected for their effectiveness in mammalian host cell expression.

Other expression systems include stable Drosophila cell transfectants and baculovirus infected insect-cells suitable for expression of proteins.

For prokaryotic host cells that do not recognize and process a native signal sequence, the signal sequence is substituted by a prokaryotic signal sequence selected, for example, from the group of the alkaline phosphatase, penicillinase, 1 pp, or heat-stable enterotoxin II leaders.

For yeast secretion the native signal sequence may be substituted by, e.g., a yeast invertase leader, a factor leader (including Saccharomyces and Kluyveromyces π-factor leaders), or acid phosphatase leader, the C. albicans glucoamylase leader, or the signal sequence described in U.S. Pat. No. 5,631,144. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter that is recognized by the host organism and is operably linked to the coding sequences. Promoters suitable for use with prokaryotic hosts include the phoA promoter, β-lactamase and lactose promoter systems, alkaline phosphatase, a tryptophan (trp) promoter system, and hybrid promoters such as the tan promoter. However, other known bacterial promoters are suitable. Promoters for use in bacterial systems also will contain a Shine-Dalgarno (S.D.) sequence operably linked to the DNA encoding the protein described herein. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding protein described herein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv (1982) NATURE, 297:17-18 on enhancing elements for activation of eukaryotic promoters. The enhancer may be spliced into the vector at a position 5′ or 3′ to the peptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of mRNA encoding the protein described herein. One useful transcription termination component is the bovine growth hormone polyadenylation region.

In some embodiments, the expression construct comprises a signal sequence operatively linked upstream from the sequences encoding the multimer composition to thereby facilitate secretion of the multimer composition from a host cell.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (1988) BIO/TECHNOLOGY, 6:47. Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides described herein would make expression in E. coli as the preferred method for expression. The protein is then purified from culture media or cell extracts.

The host cells used to produce the proteins of this disclosure may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma)) are suitable for culturing the host cells. In addition, many of the media described in Ham et al. (1979) METH. ENZYMOL., 58:44, Barites et al. (1980) ANAL. BIOCHEM., 102:255, U.S. Pat. Nos. 4,767,704, 4,657,866, 4,927,762, 4,560,655, 5,122,469, 6,048,728, 5,672,502, or U.S. Patent No. RE 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins described herein can also be produced using cell-free translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Proteins described herein can also be produced by chemical synthesis (e.g., by the methods described in SOLID PHASE PEPTIDE SYNTHESIS, 2nd Edition, The Pierce Chemical Co., Rockford, Ill. (1984)). Modifications to the protein can also be produced by chemical synthesis.

The proteins of the present disclosure can be purified by isolation/purification methods for proteins generally known in the field of protein chemistry. For example, an affinity tag (such as His6) can be incorporated into a component of the TCR multimer and the multimer can be purified by affinity chromatography (e.g., immobilized metal affinity chromatography for the His6 tag). Other non-limiting examples of suitable purification methods include immunoaffinity chromatography, extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, get filtration, gel permeation chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, or preferably at least 95% pure, and most preferably at least 98% pure. Regardless of the exact numerical value of the purity, the polypeptide is sufficiently pure for its intended use.

In certain embodiments, the expression construct includes at least one tag sequence, most typically as at the C-terminal end of the coding region, although inclusion of a tag at the N-terminal end (alternative to or in addition to the C-terminal end) is also encompassed. Suitable tag sequences are known in the art and described further herein.

Additional tags suitable for use in the methods and compositions provided herein include affinity tags, including but not limited to enzymes, protein domains, or small polypeptides which bind with high specificity to a range of substrates, such as carbohydrates, small biomolecules, metal chelates, antibodies, etc., to allow rapid and efficient purification of proteins. Solubility tags enhance proper folding and solubility of a protein and are frequently used in tandem with affinity tags. Sequences encoding such a tag(s) can be incorporated into an expression construct of the disclosure, such as at the C-terminus or N-terminus of the peptide-multimer-encoding regions to thereby incorporate a detectable tag into the expressed polypeptide.

In some embodiments, the present disclosure provides strategies for recombinantly expressing TCR multimers of the disclosure such as illustrated herein. Representative non-limiting examples of TCR multimer structures are illustrated schematically in FIGS. 1A-1C and FIGS. 2A-2C, wherein the multimers of FIGS. 2A-2C include an effector moiety (anti-CD3).

In one approach, schematically illustrated in FIG. 3A, three different constructs are used to create the TCR multimer. The first expression construct encodes TCR α chain variable (V) and constant (C) regions, operatively linked to IgM Cμ2, Cμ3 and Cμ4 constant regions and the tailpiece region, with a linker sequence positioned between the TCR and IgM sequences. Non-limiting representative amino acid and nucleotide sequences of such TCRα/Cμ constructs are shown in SEQ ID NOs: 57-60. The construct of SEQ ID NO: 57 (amino acid) and 58 (nucleotide) comprises the wild-type D046C1 TCR α chain variable (V) and constant (C) regions, operatively linked to IgM Cμ2, Cμ3 and Cμ4 constant regions and the tailpiece region, with a connecting peptide linker sequence positioned between the TCR and IgM sequences. The construct of SEQ ID NO: 59 (amino acid) and 60 (nucleotide) comprises the wild-type D046C1 TCR α chain variable (V) and constant (C) regions, operatively linked to IgM Cμ2, Cμ3 and Cμ4 constant regions and the tailpiece region, with a G-S linker sequence positioned between the TCR and IgM sequences.

Similar TCRα/Cμ constructs to those shown in SEQ ID NOs: 57-60 are shown in SEQ ID NOs: 61-64, except that in the latter constructs the multimerization moiety only contains the IgM Cμ3 and Cμ4 constant regions and the tailpiece region. Accordingly, the construct of SEQ ID NO: 61 (amino acid) and 62 (nucleotide) comprises the wild-type D046C1 TCR α chain variable (V) and constant (C) regions, operatively linked to IgM Cμ3 and Cμ4 constant regions and the tailpiece region, with a connecting peptide linker sequence positioned between the TCR and IgM sequences. Similarly, the construct of SEQ ID NO: 63 (amino acid) and 64 (nucleotide) comprises the wild-type D046C1 TCR α chain variable (V) and constant (C) regions, operatively linked to IgM Cμ3 and Cμ4 constant regions and the tailpiece region, with a G-S linker sequence positioned between the TCR and IgM sequences.

The second expression construct used in the approach shown in FIG. 3A encodes TCR β chain variable (V) and constant (C) regions. The third expression construct encodes an immunoglobulin J chain. To express the complete TCR multimer, the three expression constructs are transfected into a host cell and the host cell is cultured to allow for expression of the three encoded constructs. The TCR multimer self-assembles and can be purified from the host cells, or culture supernatant thereof, by standard methods.

The second approach, schematically illustrated in FIG. 3B, is similar to the first approach, using three separate constructs, except that the third expression construct encoding the immunoglobulin J chain is operatively linked to an anti-CD3 effector moiety, with a linker sequence positioned between the J chain and anti-CD3 sequences. Again, to express the complete TCR multimer, the three expression constructs are transfected into a host cell and the host cell is cultured to allow for expression of the three encoded constructs. The TCR multimer self-assembles and can be purified from the host cells, or culture supernatant thereof, by standard methods.

In a third approach, schematically illustrated in FIG. 3C, the same first expression construct used in the above two approaches, encoding TCR α chain V and C regions, operatively linked to IgM Cμ2, Cμ3 and Cμ4 constant regions, is used. However, rather than two separate constructs encoding the cognate TCR subunit (β chain V and C regions), for pairing with the TCR α chain, and the Ig J chain, respectively, the latter two components are encoded by the same expression construct. Thus, in this approach, the second expression vector comprises sequences encoding the TCR β chain V and C regions, as well as sequences encoding the immunoglobulin J chain, separated by a T2A ribosomal skipping sequence that allows the TCR sequences and the J chain sequences to be translated into separate proteins off the same transcript. Furthermore, an anti-CD3 effector moiety is operatively linked to the J chain sequences, with a linker positioned in between. To express the complete TCR multimer, the two expression constructs are transfected into a host cell and the host cell is cultured to allow for expression of the constructs. The TCR multimer self-assembles and can be purified from the host cells, or culture supernatant thereof, by standard methods.

### B. Chemical Conjugation Preparation of TCR Multimers

In another embodiment, a TCR multimer composition of the disclosure is produced by covalent conjugation of the TCR moiety (TM) to the multimerization moiety (MM). In certain embodiments, a multimer composition is produced by covalent conjugation of the multimerization moiety to the C-terminus of one subunit of a TCR α/β or γ/δ pair functioning as the TM.

For chemical conjugation approaches, the TM and multimerization moiety polypeptide components can be prepared separately, either recombinantly or chemically. For example, TCR V region pairings (α/β or γ/δ), optionally with C regions included, can be expressed using recombinant DNA technology by introducing an expression construct into bacterial cells, insect cells, or mammalian cells, and purifying the recombinant protein from cell extracts, e.g., as described above. The multimerization moiety polypeptide similarly can be prepared recombinantly, prior to conjugation to the TM.

A number of suitable methods for forming covalent bonds between the TCR moiety and the multimerization moiety are provided herein.

### 1. Chemical Bioconjugation

In some embodiments, the chemical conjugation can be mediated by, for example, cysteine bioconjugation. In some embodiments, the cysteine bioconjugation is mediated by cysteine alkylation. In some embodiments, the cysteine bioconjugation is mediated by cysteine oxidation. In other embodiments, the cysteine bioconjugation is mediated by a desulfurization reaction. In some embodiments, cysteine bioconjugation is mediated by iodoacetamide. In some embodiments, the cysteine bioconjugation is mediated by maleimide. Methods for utilizing cysteine mediated linkage of two moieties which can be used to produce the multimer composition disclosed herein have been described, for example, see Chalker et al. (2009) CHEM. ASIAN J., 4 (5): 630-40; Spicer et al. (2015) NAT. COMMUN., 5:4740.

In some embodiments, the multimer compositions are produced by chemical modification of amino acids other than cysteine, including but not limited to lysine, tyrosine, arginine, glutamate, aspartate, serine, threonine, methionine, histidine and tryptophan side-chains, as well as N-terminal amines or C-terminal carboxyls, as previously described (Baslé et al. (2010) M. CHEM. BIOL., 17 (3): 213-27; Hu et al. (2016) CHEM. SOC. REV., 45 (6): 1691-719; Lin et al. (2017) SCIENCE, 355 (6325): 597-602).

### 2. Native Chemical Ligation

In some embodiments, the multimer compositions are produced by native chemical ligation (NCL), wherein one polypeptide comprises a C-terminal thioester and the other polypeptide comprises an N-terminal cysteine residue, or functional equivalent thereof, wherein the reaction between the cysteine side-chain and the thioester irreversibly forms a native peptide bond, thus ligating the polypeptides together. Methods for NCL have been described in, for example, Hejjaoui et al. (2015) M. PROTEIN SCI., 24 (7): 1087-99.; Mandal et al. (2012) PROC. NATL. ACAD. SCI. USA, 109 (37): 14779-84; and Torbeev et al. (2013) PROC. NATL. ACAD. SCI. USA, 110 (50): 20051-6).

In some embodiments, β- and/or γ-thio amino acids are incorporated into the polypeptides. Desulfurization protocols can then produce the desired native side-chain. In some embodiments, non-chemical ligation is performed at an alanine residue. In other embodiments, non-chemical ligation is performed at phenylalanine, valine, leucine, threonine, lysine, proline, glutamine, arginine, tryptophan, aspartate, glutamate, and asparagine. Ligation/desulfurization approaches that remove purification steps and increase the yield of ligated products have been described.

### 3. Click Chemistry Mediated Bioorthogonal Conjugation

In some embodiments, the multimer compositions are produced by bioorthogonal conjugation between a conjugation moiety at the C-terminus of one polypeptide and a conjugation moiety at the N-terminus of the other polypeptide. As used herein, bioorthogonal conjugation refers to any chemical reaction that can occur inside of living systems without interfering with native biochemical processes, including chemical reactions that are chemical reactions that occur in vitro at physiological pH in, or in the presence of water. To be considered bioorthogonal, reactions must be selective and avoid side-reactions with other functional groups found in a given starting compound. In addition, any resulting covalent bond(s) between the reaction partners should be strong and chemically inert to biological reactions and should not affect the biological activity of the desired molecule.

In some embodiments, the bioorthogonal conjugation is mediated by “click chemistry.” (see, e.g., Kolb et al. (2001) ANGEWANDTE CHEMIE INTERNATIONAL EDITION, 40:2004-2021). The term “click chemistry”, as used herein, refers to a set of reliable and selective bioorthogonal reactions for the rapid synthesis of new compounds and combinatorial libraries. Properties of click reactions include modularity, wideness in scope, high yielding, stereospecificity and simple product isolation (separation from inert by-products by non-chromatographic methods) to produce compounds that are stable under physiological conditions. A “click reaction” can be with copper, or it can be a copper-free click reaction.

For conjugation of two polypeptides via click chemistry, click chemistry moieties of components have to be reactive with each other, for example, in that the reactive group of a click chemistry moiety on one polypeptide reacts with the reactive group of the second click chemistry moiety on the other polypeptide to form a covalent bond.

Conjugation moieties suitable for click chemistry, reaction conditions, and associated methods are available in the art (e.g., Kolb et al. (2001) ANGEWANDTE CHEMIE INTERNATIONAL EDITION, 40:2004-2021; Evans (2007) AUSTRALIAN JOURNAL OF CHEMISTRY, 60:384-395; Lahann (2009) CLICK CHEMISTRY FOR BIOTECHNOLOGY AND MATERIALS SCIENCE, John Wiley & Sons Ltd, ISBN 978-0-470-69970-6). In some embodiments, a click chemistry moiety may comprise or consist of a terminal alkyne, azide, strained alkyne, diene, dieneophile, alkoxyamine, carbonyl, phosphine, hydrazide, thiol, or alkene moiety. In certain embodiments, the azide is a copper-chelating azide. In other embodiments, the copper-chelating azide is a picolyl azide. Reagents for use in click chemistry reactions are commercially available, such as from Click Chemistry Tools (Scottsdale, AZ) or GenScript (Piscataway, NJ).

In some embodiments, sortase-mediated conjugation is used to install a first click chemistry moiety at one terminus of one polypeptide and a second click chemistry moiety at one terminus of the other polypeptide. Methods of attaching click chemistry moieties utilizing sortase are described, for example, in WO 2013/00355. In some embodiments, intein-mediated conjugation is used to install a first click chemistry moiety at one terminus of one polypeptide and a second click chemistry moiety at one terminus of the other polypeptide. Sortase-mediated and intein-mediated conjugation is described further below.

In certain embodiments, the methods of click chemistry mediated covalent conjugation of two polypeptides provided herein comprise native chemical ligation of C-terminal thioesters with β-amino thiols (Xiao J, Tolbert T J (2009) ORG. LETT., 11 (18): 4144-7).

In some embodiments, the click chemistry used to produce the multimer composition comprises 1,3-dipolar cycloaddition (e.g., the Cu(I)-catalyzed stepwise variant, often referred to simply as the “click reaction”; see, e.g., Tornoe et al. (2002) JOURNAL OF ORGANIC CHEMISTRY, 67:3057-3064). Copper and ruthenium are the commonly used catalysts in the reaction. The use of copper as a catalyst results in the formation of 1,4-regioisomer whereas ruthenium results in formation of the 1,5-regioisomer.

In some embodiments, the click chemistry conjugation comprises a cycloaddition reaction, such as the Diels-Alder reaction. In some embodiments, the polypeptides are conjugated by azide-alkyne 1,3-dipolar cycloaddition (“click chemistry”). In some embodiments, the cycloaddition is promoted by the presence of Cu(I)-catalyzed cycloaddition (CuAAC).

In some embodiments, the click chemistry conjugation comprises nucleophilic addition to small strained rings like epoxides and aziridines. In some embodiments, the cycloaddition is promoted by strained cyclooctyne systems, for example, as described in Agard et al. (2004) J. AM. CHEM. SOC., 126 (46): 15046-7.

In some embodiments, the click chemistry conjugation comprises nucleophilic addition to activated carbonyl groups.

In some embodiments, the conjugation of the polypeptides occurs by a bioorthogonal reaction. In some embodiments, the polypeptides are conjugated by inverse-electron demand Diels-Alder reactions between strained dienophiles and tetrazine dienes, for example, as described in Blackman et al. (2008) J. AM. CHEM. SOC., 130 (41): 13518-9; and Devaraj et al. (2008) BIOCONJUG. CHEM., 19 (12): 2297-9). In some such embodiments, the dienophile is a trans-cyclooctene. In some embodiments, the dienophile is a norbornene.

### 4. Sortase Mediated Conjugation

In some embodiments, conjugation between two polypeptides is mediated by a cysteine transpeptidase. In some embodiments, the cysteine transpeptidase is a sortase, or enzymatically active fragment thereof. A variety of sortase enzymes have been described and are commercially available (e.g., Antos et al. (2016) CURR. OPIN. STRUCT. BIOL., 38:111-118). Sortases recognize and cleave an amino acid motif, referred to as a “sortag”, to produce a peptide bond between the acyl donor and acceptor site on two polypeptides, resulting in the ligation of different polypeptides which contain N- or C-terminal sortags. In particular, sortases join a C-terminal LPETGG recognition motif (SEQ ID NO: 56) to an N-terminal GGG (oligoglycine) motif.

Accordingly, in some embodiments, the recognition motif is added to the C-terminus of the TCR moiety (e.g., one subunit of an α/β or γ/δ V region pair) and an oligo-glycine motif is added to the N-terminus of the multimerization moiety. Upon addition of sortase to the mixture, the two polypeptides are covalently linked through a native peptide bond.

Methods of conjugation of sortags into proteins have also been described. (Matsumoto et al. (2016) ACS SYNTH. BIOL., 5 (11): 1284-1289; Williams et al. (2016) PLOS ONE., 11 (4):e0154607; and Witte et al. (2012) PROC. NATL. ACAD. SCI. USA, 109 (30): 11993-8; Mao et al. (2004) J. AM. CHEM. SOC., 126 (9): 2670-1; Guimaraes et al. (2013) NAT. PROTOC., 8 (9): 1787-99; and Theile et al. (2013) NAT. PROTOC., 8 (9): 1800-7).

In some embodiments, the aminoglycine peptide fragment generated by the sortase reaction, is removed by dialysis or centrifugation, e.g., while the reaction is proceeding (Freiburger et al. (2015) J. BIOMOL. NMR., 63 (1): 1-8). In some embodiments, affinity immobilization strategies or flow-based platforms are used for the selective removal of reaction components (Policarpo et al. (2014) ANGEW. CHEM. INT. ED. ENGL., 53 (35): 9203-8).

In some embodiments, the equilibrium of the reaction can be controlled by ligation product or by-product deactivation, e.g., as described in Yamamura et al. (2011) CHEM. COMMUN. (CAMB)., 47 (16): 4742-4). In other embodiments, by-products are deactivated by chemical modification of the acyl donor glycine as described, for example, in Liu et al. (2014) J. ORG. CHEM., 79 (2): 487-92; and Williamson et al. (2014) NAT. PROTOC., 9 (2): 253-62).

### 5. Intein-Mediated Conjugation

Inteins are naturally occurring, self-splicing protein subdomains that are capable of excising out their own protein subdomain from a larger protein structure while simultaneously joining the two formerly flanking peptide regions (“exteins”) together to form a mature host protein. Intein-based methods of protein modification and ligation have been developed. An intein is an internal protein sequence capable of catalyzing a protein splicing reaction that excises the intein sequence from a precursor protein and joins the flanking sequences (N- and C-exteins) with a peptide bond.

As used herein, the term “split intein” refers to any intein in which one or more peptide bond breaks exists between the N-terminal intein segment and the C-terminal intein segment such that the N-terminal and C-terminal intein segments become separate molecules that can non-covalently reassociate, or reconstitute, into an intein that is functional for splicing or cleaving reactions. Any catalytically active intein, or fragment thereof, may be used to derive a split intein for use in the systems and methods disclosed herein. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing splicing reactions.

As used herein, the “N-terminal intein segment” refers to any intein sequence that comprises an N-terminal amino acid sequence that is functional for splicing and/or cleaving reactions when combined with a corresponding C-terminal intein segment. An N-terminal intein segment thus also comprises a sequence that is spliced out when splicing occurs. An N-terminal intein segment can comprise a sequence that is a modification of the N-terminal portion of a naturally occurring (native) intein sequence. For example, an N-terminal intein segment can comprise additional amino acid residues and/or mutated residues so long as the inclusion of such additional and/or mutated residues does not render the intein non-functional for splicing or cleaving. Preferably, the inclusion of the additional and/or mutated residues improves or enhances the splicing activity and/or controllability of the intein. Non-intein residues can also be genetically fused to intein segments to provide additional functionality, such as the ability to be affinity purified or to be covalently immobilized.

As used herein, the “C-terminal intein segment” refers to any intein sequence that comprises a C-terminal amino acid sequence that is functional for splicing or cleaving reactions when combined with a corresponding N-terminal intein segment. In one aspect, the C-terminal intein segment comprises a sequence that is spliced out when splicing occurs. In another aspect, the C-terminal intein segment is cleaved from a peptide sequence fused to its C-terminus. A C-terminal intein segment can comprise a sequence that is a modification of the C-terminal portion of a naturally occurring (native) intein sequence. For example, a C terminal intein segment can comprise additional amino acid residues and/or mutated residues so long as the inclusion of such additional and/or mutated residues does not render the C-terminal intein segment non-functional for splicing or cleaving.

Expressed protein ligation (EPL) refers to a native chemical ligation between a recombinant protein with a C-terminal thioester and a second agent with an N-terminal cysteine. The C-terminal thioester can readily be introduced onto any recombinant protein (i.e., the targeting ligand) through the use of auto-processing, also known as protein-splicing, mediated by an intein (intervening protein). Inteins are proteins that can excise themselves from a larger precursor polypeptide chain, utilizing a process that results in the formation of a native peptide bond between the flanking extein (external protein) fragments. When an auto-processing protein is cloned downstream of the targeting ligand, thiols (e.g., 2-mercaptoethanesulfonic acid, MESNA) can be used to induce the site-specific cleavage of the auto-processing protein, resulting in the formation of a reactive thioester. The thioester will then react with any agent that has an N-terminal cysteine. EPL operates in a site-specific manner, and the reaction is known to be very efficient if both functional groups are in high concentrations (reviewed in Elias et al. (2010) SMALL, 6:2460-2468).

Accordingly, in some embodiments, one polypeptide component of the TCR multimer is ligated to an alkynated peptide by expressed protein ligation (EPL) and then conjugated to an azide-labeled second polypeptide of the multimer by Cu(I)-catalyzed terminal azide-alkyne cycloaddition (CuAAC).

A number of inteins have now been described including, but not limited to MxeGyrA (Frutos et al. (2010); Southworth et al. (1999); SspDnaE (Shah et al. (2012); Wu et al. (1998); NpuDnaE (Shah et al. (2012); Vila-Perello et al. (2013); AvaDnaE (David et al. (2015); Shah et al. (2012); Cfa (consensus DnaE split intein) (Stevens et al. (2016)); gp41-1 and gp41-8 (Carvajal-Vallejos et al. (2012)); NrdJ-1 (Carvajal-Vallejos et al. (2012)); IMPDH-1 (Carvajal-Vallejos et al. (2012)) and AceL-TerL (Thiel et al. (2014)).

Suitable intein sequences and protocols for use in protein conjugation have been described in the art, such as in Stevens et al. (2016) J. AM. CHEM. SOC., 138, 2162-2165; Shah et al. (2012) J. AM. CHEM. SOC., 134, 11338-11341; and Vila-Perello et al. (2013) J. AM. CHEM. SOC., 135, 286-292; Batjargal et al. (2015) J. AM. CHEM. SOC., 137 (5): 1734-7; and Guan et al. (2013) BIOTECHNOL. BIOENG., 110 (9): 2471-81.

### 6. Additional Bioconjugation Methods

In some embodiments, the conjugation of the polypeptides of the TCR multimer is mediated enzymatically. In some embodiments, the enzyme is formylglycine generating enzyme (FGE) that recognizes the CXPXR amino acid sequence motif and converts the cysteine residue to formylglycine, thus introducing an aldehyde functional group (Wu et al. (2009) PROC. NATL. ACAD. SCI. USA, 106 (9): 3000-5), which is subjected to bio-orthogonal transformations such as oximation and Hydrazino-Pictet-Spengler reactions (Agarwal et al. (2013) BIOCONJUG. CHEM., 24 (6): 846-51; Dirksen et al. (2008) BIOCONJUG. CHEM., 19 (12): 2543-8).

Site-specific bioconjugation strategies offer many possibilities for directed protein modifications. Among the various enzyme-based conjugation protocols, formylglycine-generating enzymes allow to post-translationally introduce the amino acid Cα-formylglycine (FGly) into recombinant proteins, starting from cysteine or serine residues within distinct consensus motifs. The aldehyde-bearing FGly-residue displays orthogonal reactivity to all other natural amino acids and can, therefore, be used for site-specific labeling reactions on protein scaffolds. (Reviewed in Kruger et al. (2019) BIOL. CHEM., 400 (3): 289-297).

Formylglycine generating enzyme (FGE) recognizes a pentapeptide consensus sequence, CxPxR, and it specifically oxidizes the cysteine in this sequence to an unusual aldehyde-bearing formylglycine. The FGE recognition sequence, or aldehyde tag, can be inserted into heterologous recombinant proteins produced in either prokaryotic or eukaryotic expression systems. The conversion of cysteine to formylglycine is accomplished by co-overexpression of FGE, either transiently or as a stable cell line, and the resulting aldehyde can be selectively reacted with α-nucleophiles to generate a site-selectively modified bioconjugate (Rabuka et al. (2012) NAT. PROTOC., 7 (6): 1052-1067).

In some embodiments, the enzyme is lipoic acid ligase, an enzyme that modifies a lysine side-chain within the 13-residue target sequence (Uttamapinant et al. (2010) Proc. Natl. Acad. Sci. USA, 107 (24): 10914-9) to introduce bio-orthogonal groups, including azides, aryl aldehydes and hydrazines, p-iodophenyl derivatives, norbornenes, and trans-cyclooctenes (reviewed in Debelouchina et al. (2017) Q. REV. BIOPHYS., 50 e7. doi: 10.1017/S0033583517000021). In some embodiments, the enzyme is biotin ligase, farnesyltransferase, transglutaminase or N-myristoyltransferase (reviewed in Rashidian et al. (2013) BIOCONJUG. CHEM., 24 (8): 1277-94).

### IV. Pharmaceutical Compositions and Kits

The TCR multimers can be incorporated into a pharmaceutical composition, formulated together with a pharmaceutically acceptable carrier. Such compositions may include one or a combination of (e.g., two or more different) TCR multimers. The TCR multimer pharmaceutical composition also can be used in combination with other therapeutics agents, or other therapeutic modalities, in a subject (so-called combination therapy that combines two or more treatment approaches).

Preferably, in some embodiments the carrier is suitable for intravenous, intramuscular, subcutaneous, parenteral, spinal or epidermal administration (e.g., by injection or infusion).

The pharmaceutical composition also may include one or more pharmaceutically acceptable salts. A “pharmaceutically acceptable salt” refers to a salt that retains the desired biological activity of the parent compound and does not impart any undesired toxicological effects (see e.g., Berge et al. (1977) J. PHARM. SCI., 66:1-19). Non-limiting examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The pharmaceutical composition also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, α-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The pharmaceutical compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

Pharmaceutical compositions can be administered via one or more routes of administration using one or more of a variety of methods known in the art. As will be appreciated by the skilled artisan, the route and/or mode of administration will vary depending upon the desired results. Preferred routes of administration for TCR multimers of the disclosure include intratumoral, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intratumoral, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In another aspect, the disclosure provides kits comprising a TCR multimer, e.g., for use in the methods described herein. Any of the compositions of the disclosure can be formulated into a kit, including the components, typically packaged in one or more containers, along with instructions for use of the components for the desired use. For example, in certain embodiments, the disclosure provides a kit comprising at least one TCR multimer composition packaged in a container, along with instructions for use of the multimer, e.g., for detecting pMHC or for immunomodulation. In another embodiment, the kit comprises at least one expression construct for expressing a TCR multimer of the disclosure, typically packaged in a container, along with instructions for use of the construct(s), e.g., for expression of a TCR multimer in a host cell. In some embodiments, a kit comprises components for constructing a multimer, such as, e.g., one or more plasmids encoding one or more constructs as provided herein, that can be transfected into a host to generate a multimer.

### V. Methods of Use

The TCR multimers of the disclosure can be used in a variety of in vitro and in vivo methods, as research reagents, for diagnostic purposes, and/or for therapeutic uses, based on the binding specificity of the TCR multimers and on the effector functions of the TCR multimers.

Since the TCR moiety of a provided multimer recognizes cognate peptide-MHC, in some embodiments the multimers can be used to detect peptide-MHC complexes. Accordingly, in another aspect, the disclosure pertains to a method of detecting a peptide-MHC complex, the method comprising contacting a peptide-MHC complex with a TCR multimer of the disclosure and detecting binding of the TCR multimer to the peptide-MHC complex to thereby detect the peptide-MHC complex. In some embodiments, TCR multimers are used to treat a subject with a particular disease or condition. For example, in some embodiments, TCR multimers are administered to a subject with cancer, an infectious disease, or an autoimmune disease (e.g., such as an autoimmune disease characterized by presence of an autoantigen presented by MHC molecules).

A T cell refers to a type of white blood cell that can be distinguished from other white blood cells by the presence of a T cell receptor on the cell surface. T cells can be from a T cell subpopulation defined by, e.g., function, activation state, maturity, potential for differentiation, expansion, recirculation, localization, and/or persistence capacities, antigen specificity, type of antigen receptor, presence in a particular organ or compartment, cell surface marker or cytokine secretion profile, and/or degree of differentiation. For example, in some embodiments subpopulations of T cells are naive T (Tn) cells, effector T cells (Teff), memory T cells and sub-types thereof, such as stem cell memory T (Tscm), central memory T (Tcm), effector memory T (Tem), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells (e.g., TH1 cells, TH2 cells, TH3 cells, TH17 cells, TH9 cells, TH22 cells, follicular helper T cells), natural killer T (NKT) cells, alpha(α)/beta(β) T cells, and gamma(γ)/delta(δ) T cells. T cells can be characterized by the expression of certain cell surface molecules such as the T cell receptor (TCR), a multiprotein complex that is involved in MHC binding.

As will be understood by those of skill in the art, there are several subsets of T cells, including, but not limited to, T helper cells (a.k.a. T_{H} cells or CD4⁺ T cells) and subtypes, including T_{H}1, T_{H}2, T_{H}3, T_{H}17, T_{H}9, and T_{FH} cells, cytotoxic T cells (a.k.a T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ Treg cells, CD4⁺ FOXP3⁻ T_{reg} cells, Tr1 cells, Th3 cells, and T_{reg}17 cells, natural killer T cells (a.k.a. NKT cells), mucosal associated invariant T cells (MAITs), and gamma (γ) delta (δ) T cells (γδ T cells), including Vγ9/Vδ2 T cells. The term “T cell cytotoxicity” includes any immune response that is mediated by CD8+ T cell activation.

### A. MHC Localization and Specificity

In some embodiments, the peptide-MHC complex is on the surface of a cell such as an antigen presenting cell (APC) and the TCR multimer is contacted with the cell (e.g., an APC such as a T cell or a cancer cell). In another embodiment, the peptide-MHC complex is fixed to a solid support, such as a plate or a bead. In yet another embodiment, the peptide-MHC complex is soluble. Binding of the TCR multimer to the peptide-MHC complex can be detected by standard methods known in the art, e.g., by labeling the TCR multimer with a detectable label and then detecting the amount of label associated with the peptide-MHC complex. Alternatively, the peptide-MHC complex can be labeled with a detectable label and then the amount of label associated with the TCR multimer can be detected.

In some embodiments, the TCR moiety of the multimer has a known antigen/MHC specificity and the method can be used to detect that particular peptide-MHC complex in a sample, e.g., for research purposes or on the surface of patient cells for the purposes of characterizing a patient's antigenic presentation capacity or diagnosing a patient as having a tumor that expresses a particular cancer antigen/MHC complex, or a patient having an infection or autoimmune disease that is associated with a particular infectious antigen or autoantigen/MHC complex.

In another embodiment, the detection method can be used to analyze the binding specificity of the TCR moiety in the multimer. For example, a TCR V region α/β or γ/δ pair whose antigenic specificity is unknown can be used in the TCR moiety to determine the binding specificity of the TCR moiety to cognate pMHCs by using the detection method. Alternatively, a particular TCR V region pair having a known antigen specificity can be used in the detection method to investigate possible antigen crossreactivity of that particular TCR V region pair. To analyze the binding specificity of a TCR multimer, the multimer can be used to screen peptide-MHC libraries having a large diversity of peptide-MHC complexes. Such peptide-MHC libraries, and screening thereof, are known in the art.

### B. Targeting Specific Peptide-MHC Complexes

The present disclosure provides the insight that TCR multimers as disclosed herein can be coupled to effector moieties to modulate an immune response. In some embodiments, TCR multimers coupled to effector moieties target cognate pMHC complexes on target cells acting in cis (i.e., TCR multimers directly acting on a population of cells such as tumor cells or T cells driving inflammation). In some embodiments, TCR multimers coupled to effector moieties target cognate pMHC complexes on target cells acting in trans (i.e., TCRs multimers indirectly acting on target cells that present the cognate pMHC complexes, through intermediary cells, e.g., T cells or NK cells, that are present in the microenvironment or engaged to the target cells via the TCR multimer or otherwise).

Targeting one or more tissues, cell populations, or immune cells may comprise use of TCR multimers provided herein to target cognate pMHC complexes. In some such embodiments, TCR multimers may be coupled to one or more effector moieties. Effector moieties can comprise for example cellular toxins or cell death moieties, T cell activation moieties, T cell inhibition moieties, and immune cell recruitment moieties e.g., T cell recruitment moieties or NK cell recruitment moieties.

Particular tissues and cell populations to be targeted and effector moieties chosen will depend on context and desired outcome. In some embodiments, a population of cells in a tissue under attack in an autoimmune disease expressing certain cognate pMHCs (e.g., autoantigen expressing cells, etc.) may be targeted by TCR multimers comprising effector moieties that drive autoreactive T cells into inhibition and/or exhaustion. In some embodiments, a population of cells is infected (e.g., with a virus). In some embodiments, a population of cancer cells expressing certain cognate pMHCs (e.g., antigen-specific cancer cells, etc.) may be targeted by TCR multimers for cell death and/or elimination. In some embodiments, a population of immune cells including but not limited to effector T cells expressing certain cognate pMHCs (e.g., antigen-specific T cells, etc.) may be targeted by TCR multimers for inhibition and/or exhaustion, cell death and/or elimination, T cell function support, and/or cell expansion. For example, without being limited by theory, the present disclosure contemplates that, in some embodiments, an infected T cell (e.g., a T cell infected with a virus such as HIV), could be targeted using TCR multimers as provided herein to recognize viral epitopes expressed on, e.g., Class I alleles of an infected T cell.

For instance, the present disclosure contemplates that, in some embodiments, a strategy for employing TCR multimer-mediated inhibition or activation of effector T cells can be achieved by providing suppressive or activating signals, depending on context.

Without wishing to be bound by any particular theory, in some embodiments, such immune suppressive signals may be useful in modulating autoreactive T cells (e.g., effector T cells, helper T cells) that attack a self tissue by targeting a TCR multimer with a effector moiety to tissue presenting cognate pMHCs such that the autoimmune disease is treated. In some embodiments, for example, the TCR multimer recognizes and binds a cognate pMHC on cells in a tissue presenting autoreactive peptides, and the effector moiety may bind to a cognate epitope on a autoreactive T cell and act to stop further (i) activity and/or (ii) recruitment of T cells, such that the tissue under self-attack is shielded from continued attack. For example, immune suppression by inducing and maintaining tolerance in autoimmunity by TCR monomers having a different structure than those provided by the present disclosure have been described in the art (see e.g., Curnock et al. (2021) JCI INSIGHT, 6:3152468).

The present disclosure also contemplates that in some such embodiments, certain cells (e.g., tumor cells, infected cells, such as virally-infected cells), may be targeted for death. In some embodiments, the TCR multimer recognizes and binds cognate pMHCs on tumor cells or infected cells (e.g., virally-infected cells), and the effector moiety may be a cellular toxin or cell death moiety. For example, in some embodiments, a TCR multimer comprising a cytotoxic effector moiety may be targeted to a tumor cell or an infected cell, and, via binding to its cognate MHC, be internalized into the cell, thereby killing it and treating the cancer or infection. In some embodiments, a population of immune effector cells (e.g., cytotoxic T cells, NK cells) may be recruited to a tumor cell to cause tumor cell death or to an infected cell to kill the infected cell, such as by targeting TCR multimers recognizing and binding a cognate pMHC on tumor cells or infected cells, and recruiting an immune effector cell via a binding moiety to such cells as the effector moiety.

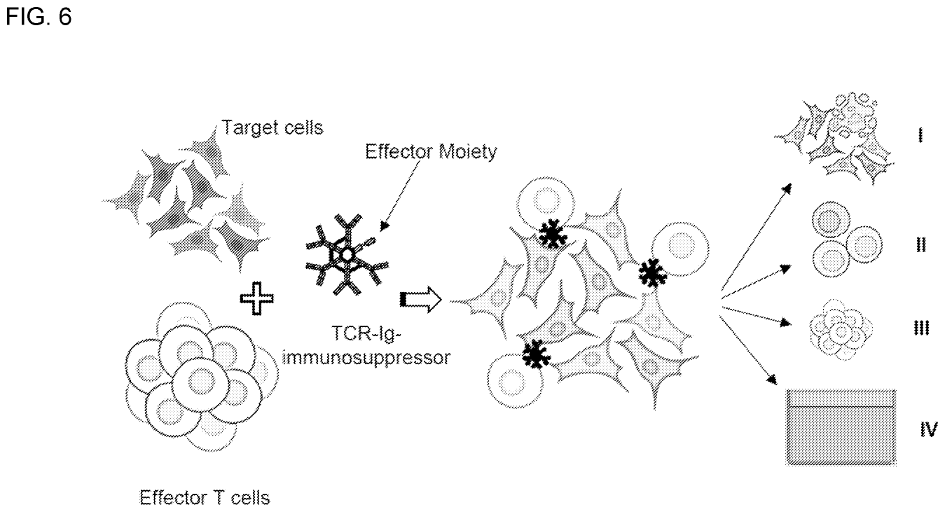

In some embodiments, cells or responses to TCR multimers may be characterized by one or more evaluation strategies such as, e.g., differential labeling techniques to determine numbers of cell types (e.g., such as using a discriminating anti-HLA label to determine relative numbers of each cell type via, e.g., flow cytometry). Another exemplary strategy to evaluate cells is to use markers such as Annexin V or propidium iodide, and/or perform counts and/or determine relative amounts of certain populations of T cells (antigen-specific and antigen non-specific) to determine if apoptosis has selectively and/or successfully been induced in a targeted population. Additional examples of assessment of cellular responses to TCR multimers as provided herein may be performed by, among other things, evaluating ability to impact TCR signaling, proliferation, cytokine secretion, or cell killing by TCR-multimer targeted cells, as illustrated in an exemplary schematic (see FIG. 6) and further described in Example 4.

### 1. Therapeutic TCR Multimers for Treating Cancer

i. Killing or Inducing Apoptosis of Tumor Cells by Cis TCR-Mediated Targeting

In some embodiments, tumor cells may be targeted to induce cell death, whether by apoptosis, cell toxicity, or other mechanisms. For example, by using a TCR multimer comprising an effector moiety where the effector moiety triggers or causes apoptosis or cell toxicity, DNA damage (e.g., inducing programmed cell death), or causes death by another pathway, tumor cells can be eliminated. For example, in some embodiments, a TCR multimer may comprise an effector moiety comprising FasL, an anti-Fas agonist antibody or antibody domain, a tubulin inhibitor, a DNA damaging agent, or any or any portion or component thereof that can bind or be delivered to act on a targeted tumor cell.

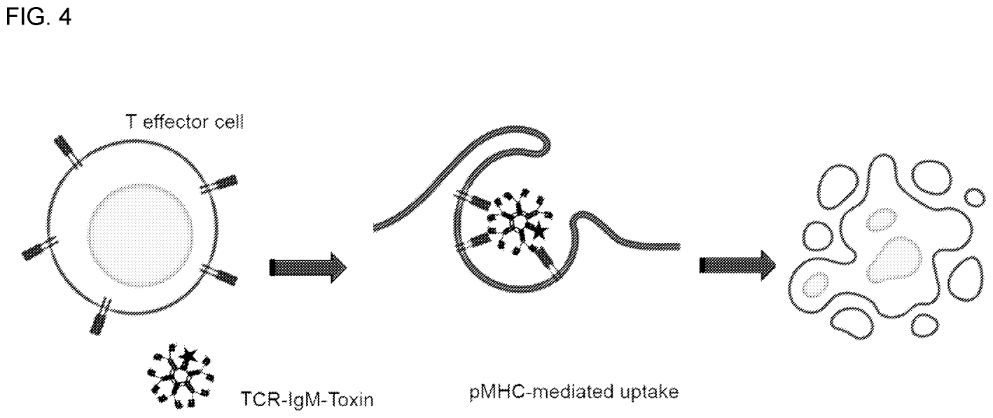

To give but one example, TCR multimers comprising an effector moiety comprising a tubulin inhibitor can be used to trigger apoptosis in tumor cells. In such an example, a TCR multimer coupled to a tubulin inhibitor can be delivered to a site of a tumor (either directly or systemically). Tumor cells should be particularly targeted for delivery of the effector moiety on the TCR multimer via the TCR multimer's cognate pMHC expressed on the tumor cell, thus internalizing the cytotoxic molecule, and specifically eliminating tumor cells (see e.g., FIG. 4). It is also possible to conduct in vitro tests using biopsied or excised tumor cells. For example, a TCR-effector moiety multimer can be co-incubated with tumor cells and non-tumor cells. The cells can then be evaluated using exemplary strategies such as differential labeling techniques to determine numbers of cell types (e.g., such as using a discriminating anti-HLA label to determine relative numbers of each cell type via, e.g., flow cytometry). Another exemplary strategy to evaluate cells is to use markers such as Annexin V or propidium iodide, and/or perform counts and/or determine relative amounts of certain populations of T cells (antigen-specific and antigen non-specific) to determine if apoptosis has selectively and/or successfully been induced in a targeted population.

ii. Killing Tumor Cells by Trans TCR-Mediated Targeting to Recruit Other Immune Cell Populations

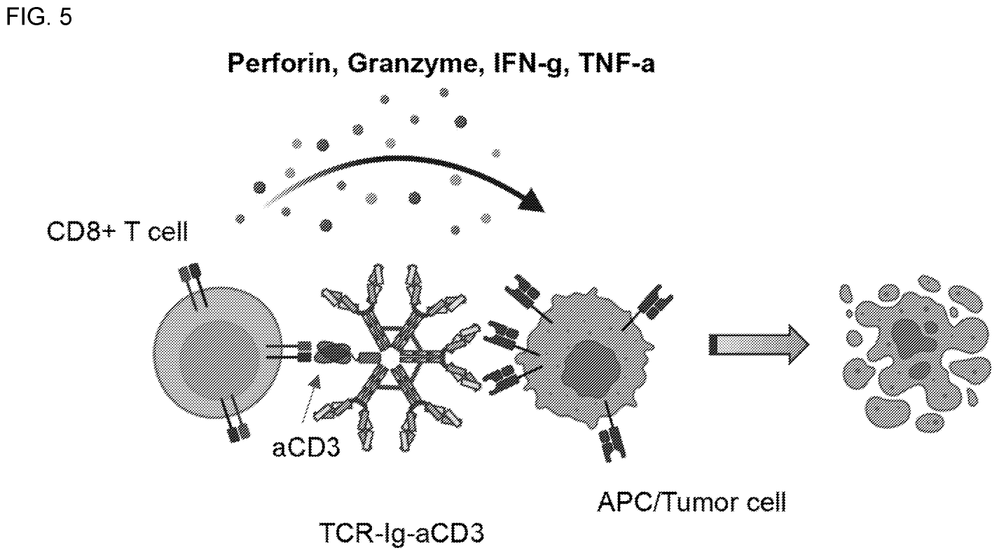

In some embodiments, tumor cells may be depleted by using a TCR multimer comprising an effector moiety that binds an immune effector cell (e.g., T cell such as CD8+ T cell, NK cell). In this trans approach, which indirectly acts on a tumor cell by facilitating the formation of an immunological synapse between an immune effector cell and a TCR-targeted cell (e.g., a tumor cell, an APC), thereby recruiting the immune effector cell to kill the TCR-targeted cell. For example, in some embodiments, the effector moiety comprises a binding domain (e.g., an antibody or an antigen-binding fragment thereof) that binds a T cell surface protein, such as CD3, CD2, or CD8 (e.g., anti-CD3 antibody, anti-CD2 antibody, or anti-CD8 antibody). In a specific embodiment, the effector moiety comprises an anti-CD3 antibody or an antigen-binding fragment thereof, thereby to produce a TCR-αCD3 multimer. Such TCR multimers can engage effector T cells to tumor cells, where the TCR multimer is targeted to the tumor cells, and the anti-CD3 domain engages other T cells (e.g., effector T cells), which kill the tumor cell via secretion of perforin, granzyme and other mechanisms (e.g., as shown in FIG. 5). Differential labeling of the different cells (with CFSE or, for instance, a discriminating anti-HLA antibody) can be used to determine relative number of each cell type by flow cytometry, and the loss of the cancer cells caused by the TCR-αCD3 multimer can be assessed. In some embodiments, the effector moiety comprises a binding domain (e.g., an antibody or an antigen-binding fragment thereof) that binds an NK cell surface protein, such as NKp46, CD16a, CD56, NKG2D, NKp30, or CD64 (e.g., anti-NKp46 antibody, anti-CD16a antibody, anti-CD56 antibody, anti-NKG2D antibody, anti-NKp30 antibody, or anti-CD64 antibody). Such TCR multimers can engage NK cells to target cells (e.g., tumor cells, APCs) to kill the target cell via secretion of perforin, granzyme and other mechanisms.

iii. Targeting T Effector Cells by Trans TCR-Mediated Targeting to Stimulate T Cell Function

In some embodiments, tumor cells may be depleted by using a TCR multimer comprising an effector moiety that enhances T cell function. For example, without being limited by theory, the present disclosure contemplates that, among other things, a TCR multimer may bind to a receptor on MHC in a tumor microenvironment, which may, in some embodiments, relieve macrophage-induced exhaustion and allow T cells to function (rather than, e.g., to continue to be chronically exhausted and unable to attack tumor cells). In some embodiments, it may be possible to block T cells from exhausted phenotypes when a cancer is detected earlier (i.e., prior to robust exhaustion), thereby mitigating the cancer progression.

In some embodiments, the effector moiety comprises a checkpoint protein antagonist such as an antagonist to PD-1, CTLA4, Lag3, TIM3, TIGIT, LILRB1, LILRB2 or an antigen-binding fragment thereof (e.g., anti-PD-1 antagonist antibody, anti-CTLA4 antagonist antibody, anti-Lag3 antagonist antibody, anti-TIM3 antagonist antibody, anti-TIGIT antagonist antibody, anti-LILRB1 antagonist antibody, or anti-LILRB2 antagonist antibody). In some embodiments, TCR multimers comprising such an effector moiety can stimulate T cell function in a trans mechanism such as by blocking the ability of cells to bind the checkpoint proteins and exhaust T cells from functioning against the cancer. In some embodiments, a TCR multimer may comprise an effector comprising an anti-inflammatory cytokine such as, e.g., IL10, TGFβ, or HLA-G (extracellular region). In some such embodiments, the present disclosure contemplates that it may be possible to promote Treg cells in order to act, for example, as a vaccine against a cancer.

In some embodiments, responses can be characterized using techniques such as differential labeling of cell populations (e.g., with CFSE or, for instance, a discriminating anti-HLA antibody) in order to determine relative number of each cell type present by, e.g., flow cytometry. Using such techniques, or others known to those of skill in the art, stimulation of some populations of T cells or loss of cancer cells due to stimulation by way of TCR multimer-effector moiety impact can be assessed.

iv. Targeting Treg Cells by Trans TCR-Mediated Targeting for Reprogramming

In some embodiments, Treg cells located in a tumor and/or in a tumor microenvironment can be reprogrammed to overcome the immunosuppressive tumor microenvironment to deplete tumor cells. This specific targeting of Tregs in the tumor and/or the tumor microenvironment has the advantage that the Treg cells are not targeted in a systemic way, but just localized to the tumor and/or the tumor microenvironment thereby avoiding triggering autoimmune disease after tumor treatment. For example, in some embodiments, a TCR multimer may comprise an effector moiety comprising IL-2 or IL2 muteins (e.g., those with reduced or no binding affinity for CD25/IL2Rα), IL-12, IL-15, or TGFβ that may reprogram Treg cells from producing immunosuppressive cytokines, to producing pro-inflammatory cytokines, thus supporting a pro-inflammatory environment for effector T cells to eliminate tumor cells.

### 2. Therapeutic TCR Multimers for Treating Infection

i. Killing Infected Cells by Trans TCR-Mediated Targeting to Recruit Other Immune Cell Populations

In some embodiments, infections may be treated by attacking a population of infected cells, such as cells infected with a virus. In some such embodiments, infected cells (e.g., virally infected cells) may be depleted by using a TCR multimer comprising an effector moiety that binds an immune effector cell (e.g., T cell such as CD8+ T cell, or NK cell). This trans approach indirectly acts on the infected cell (e.g., virally-infected cell) engaging an immune effector cell with a target (e.g., virally infected cell), thereby recruiting the immune effector cell to kill the target. The present disclosure contemplates that in some such embodiments, targeted killing of infected cells limits viral replication in order to treat the virus (e.g., by stopping it from continuing to infect additional cells).

In some embodiments, the effector moiety comprises a binding domain (e.g., an antibody or an antigen-binding fragment thereof) that binds a T cell surface protein, such as CD3, CD2, or CD8 (e.g., an anti-CD3 antibody, an anti-CD2 antibody, or an anti-CD8 antibody, or a fragment thereof). In some embodiments, the effector moiety comprises a binding domain (e.g., an antibody or an antigen-binding fragment thereof) that binds an NK cell surface protein, such as NKp46, CD16a, CD56, NKG2D, NKp30, or CD64. Such TCR multimers can engage T cells or NK cells to kill the target, for example, via secretion of perforin, granzyme and other mechanisms where the target is an eukaryotic or prokaryotic cell.

ii. Killing or Inducing Apoptosis of Virus Infected Cells by Cis TCR-Mediated Targeting

In some embodiments, virus infected cells may be targeted to induce cell death, whether by apoptosis, cell toxicity, or other mechanisms. For example, by using a TCR multimer comprising an effector moiety where the effector moiety triggers or causes apoptosis or cell toxicity, DNA damage (e.g., inducing programmed cell death), or causes death by another pathway, tumor cells can be eliminated. For example, in some embodiments, a TCR multimer may comprise an effector moiety comprising FasL, an anti-Fas agonist antibody or antibody domain, a tubulin inhibitor, a DNA damaging agent, or any or any portion or component thereof that can bind or be delivered to act on a targeted viral infected cell.

Assessment of cellular responses may be performed by, among other things, evaluating ability to impact TCR signaling, proliferation, cytokine secretion, or cell killing by TCR-multimer targeted cells, as illustrated in an exemplary schematic (see FIG. 6) and further described in Example 4.

### 3. Therapeutic Multimers for Treating Autoimmune Disease

i. Inhibiting and/or Exhausting T Effector Cells by Trans TCR-Mediated Targeting

In some embodiments, the present disclosure provides technologies that can be used to treat autoimmune disease.

For example, the present disclosure provides technologies that, in the context of autoimmune disease, where an inappropriate attack on self-cells occurs, an immune response can be modulated using a TCR multimer comprising an effector moiety to deplete, anergize or exhaust effector T cells that are attacking the tissue by targeting to particular MHCs.

For example, in a context of autoimmunity, an autoantigen or an epitope thereof may be targeted using a TCR multimer coupled to an effector moiety. The present disclosure contemplates that using a TCR multimer comprising an effector moiety, which effector moiety comprises an immunosuppressive agent can, in some embodiments, target the immunosuppressor to self cells (e.g., an APC) and/or tissue, thereby reducing the activity of the autoreactive T cells that may attack them.

As such, in some embodiments, the TCR multimer effector moiety is localized to a population of cells by the TCR multimer binding to its cognate pMHC on cells presenting autoantigens, whereas the effector moiety binds and inhibits auto-reactive T cells (i.e., those attacking self-tissue) from (i) continuing to attack and/or (ii) from any additional cells being recruited to attack the tissue. For example, autoreactive T cells can be driven to exhaustion by contacting activating effector moieties to the T cells. Furthermore, the present disclosure contemplates that since the TCR moiety of the multimer does not directly mediate any downstream T cell signaling such as would occur, for example, in a T cell expressing a corresponding full-length TCR, the TCR multimer comprising an effector moiety may also neutralize the cognate epitope in the autoantigen and act as a shield to the inflamed tissue, protecting it from further attack.

In some embodiments, specific tissues are targeted using a TCR multimer coupled to an effector moiety as provided herein. In some such embodiments, the effector moiety comprises one or more of a checkpoint protein agonist, a TNFR superfamily agonist (e.g., a TNF superfamily member), or an anti-inflammatory cytokine, or binding fragment (e.g., an antigen-binding fragment) thereof. For example, in some embodiments, an effector moiety may be or comprise a PD-L1 extracellular domain or domain 1, an anti-PD1 agonist antibody or domain thereof, an anti-CTLA4 agonist antibody or domain thereof, an anti-Lag3 agonist antibody or domain thereof, an anti-TIM3 agonist antibody or domain thereof, anti-TIGIT agonist antibody or domain thereof, anti-TNFR2 agonist antobody, 4-1BBL (CD137L) or a domain thereof, anti-CD137 agonist antibody or domain thereof, HLA-G, anti-LILRB1 agonist antibody or domain thereof, anti-LILRB2 agonist antibody or domain thereof, IL10, TGFβ or an antigen-binding fragment thereof (e.g., that can bind and/or act on a targeted T cell). In another embodiment, the effector moiety comprises a PD1 agonist, where the agonist is PDL1 ECD or a domain thereof, or an anti-PD1 antibody or a domain thereof. Without wishing to be bound by theory, the present disclosure contemplates that as the TCR-PD1 agonist multimers target the autoreactive pMHC-presenting cells, the effector moiety (e.g., PDL1 or PD1 agonist antibody) may inhibit any incoming autoreactive T cells by engaging their receptors (e.g., PD1 receptors) to induce exhaustion of target T cells. Similarly, in some embodiments, effector moieties such as, e.g., IL-10. HLA-G fusions, or anti-LILRB1 or anti-LILRB2 agonists, or, e.g., TNFR Superfamily Protein-related effectors such as 4-1BBL (CD137L), α-CD137 agonist antibody, or α-TNFR2 agonist antibody, may act to cause immunosuppression.

Assessment of cellular responses may be performed by, among other things, evaluating ability to impact TCR signaling, proliferation, cytokine secretion, or cell killing by TCR-multimer targeted cells, as illustrated in an exemplary schematic (see FIG. 6) and further described in Example 4.

### 4. Therapeutic Proteins for Treating Autoimmune Diseases, Infectious Diseases, or Cancer

i. Combinations for Targeting Multiple Populations

The present disclosure also contemplates that targeting one or more populations of T cells using distinct mechanisms with distinct effector moieties can be accomplished using multiple compositions, each composition comprising a distinct TCR multimer formulation. For example, in some embodiments, one population of T cells may be targeted for expansion and one targeted for cell death and each targeted by a distinct TCR multimer coupled to an effector moiety that is capable of supporting or killing particular T cells (e.g., in cis and/or in trans). In some embodiments, such an approach may be used in treatment of autoimmune diseases, infectious diseases, or in cancers.

Assessment of cellular responses may be performed by, among other things, evaluating ability to impact TCR signaling, proliferation, cytokine secretion, or cell killing by TCR-multimer targeted cells, as illustrated in an exemplary schematic (see FIG. 6) and further described in Example 4.

### C. Administration

When used therapeutically, the TCR multimers can be administered to a subject (e.g., intravenously or intramuscularly) to modulate an immune response in the subject. Accordingly, in another aspect, the disclosure pertains to a method of modulating an immune response in a subject, the method comprising administering to the subject the TCR multimer of the disclosure such that an immune response is modulated in the subject. Modulation of an immune response in the subject results from the combination of the binding of the TCR moiety to its target peptide-MHC complex in a multivalent manner (due to the multivalency of the multimer) and the effector functions mediated by the IgM or IgA C regions within the multimerization domain, such as complement-mediated cytotoxicity, as well as any effector functions mediated by an effector moiety and/or additional functional moiety included in the TCR multimer. For example, inclusion of a CD3-binding effector moiety serves to redirect endogenous T cells to the TCR multimers or TCR-multimer-bound target cell in vivo; the endogenous T cells are then activated in the subject, thereby modulating an immune response.

In some embodiments, compositions provided by the present disclosure are administered in vivo or ex vivo. In some embodiments, compositions are administered subcutaneously, intramuscularly, or intravenously. In some embodiments, compositions are administered by targeted injection into a particular site (e.g., an inflamed or infected tissue or organ, e.g., a tumor).

In certain embodiments, a method or composition provided herein, is administered in combination with one or more additional therapies, e.g., surgery, radiation therapy, anti-inflammatory therapy, anti-viral therapies, biologic therapy, etc. In some embodiments, the additional therapy may include chemotherapy, e.g., a cytotoxic agent. In some embodiments the additional therapy may include a targeted therapy, e.g., a tyrosine kinase inhibitor, a proteasome inhibitor, or a protease inhibitor. In some embodiments, the additional therapy may include an anti-inflammatory, anti-angiogenic, anti-fibrotic, or anti-proliferative compound, e.g., a steroid, a biologic immunomodulator, a disease modifying anti-rheumatic drug, a monoclonal antibody, an antibody fragment, an aptamer, an siRNA, an antisense molecule, a fusion protein, a cytokine, a cytokine receptor, a bronchodilator, a statin, an anti-inflammatory agent (e.g., methotrexate), or an NSAID. In some embodiments, the additional therapy may include an anti-viral agent or an antibiotic. In certain embodiments, the additional therapy may include a combination of therapeutics of different classes.

In some embodiments, one or more agents administered in combination with one another may be administered in one or more doses. In some embodiments, administration of a combination of two or more agents can be sequential or concomitant.

In some embodiments, a TCR multimer of the disclosure is administered to a subject with cancer, an infectious disease, and/or an autoimmune disease.

In some embodiments, a TCR multimer is administered to a subject with cancer and modulates the immune response to the cancer in the subject. In some embodiments, the present disclosure provides a method of treating cancer using an immunotherapy comprising a TCR multimer comprising an effector moiety. In some such embodiments, the effector moiety modulates one or more immune cell populations to treat the cancer, such as by amplifying an anti-tumor response or depleting or suppressing tumor cells, such as, e.g., by directly binding to and killing a tumor cell, or by recruiting cells to kill tumor cells. Accordingly, in some embodiments, the method comprises administering to the subject a TCR multimer of the disclosure wherein the TCR moiety of the TCR multimer binds to a cognate pMHC on target T cells (e.g., cancer-specific T cells) of the subject. In some embodiments, the TCR multimer includes an effector moiety. In some such embodiments, the effector moiety is an activating agent, e.g., an agent that expands tumor-reactive T cells. Non-limiting examples of such activating targeting moieties include IL-2 or muteins thereof such as an IL2 mutein that has reduced/no binding affinity for the CD25/IL2Rα receptor, IL12, IL15, anti-CD28 antibody domain, anti-CD40L antibody domain, anti-4-1BB antibody domain, CD80, and 4-1BBL. In another embodiment, the effector moiety is an inhibitory agent, e.g., an immunosuppressive agent and/or an agent that selectively inhibits Tregs. Non-limiting examples of such inhibitory moieties include PD-L1, FasL, TGFβ, and others provided herein. Additionally or alternatively, the subject can be treated with additional anti-cancer agents, including chemotherapeutic agents, immunotherapy agents, and immune checkpoint inhibitors.

The present disclosure also contemplates that administration of a TCR multimer as provided herein can modulate an autoimmune response in a subject with an autoimmune disease or disorder by downregulation of immunosuppression of the autoimmune response. As previously described, downregulation of autoimmunity by TCR monomers have been described in the art (see e.g., Curnock et al. (2021) JCI INSIGHT, 6:3152468). The present disclosure provides the insight that compositions comprising TCR multimers as provided herein can use TCR moieties to specifically target effector moieties to tissues that express autoantigens. In some embodiments, such specific targeting improves efficacy of treatment as compared to TCR monomers, and decreases potential off target effects (i.e., by avoiding non-specific or less-specific targeting than can occur with a TCR monomer as compared to TCR multimers of the present disclosure).

Accordingly, among other things, the present disclosure provides methods comprising administering a TCR multimer comprising an effector moiety to a subject in need thereof. In some embodiments, a method comprises administering to the subject a TCR multimer in accordance with the present disclosure, wherein the TCR moiety of the multimer targets tissues expressing autoantigens and the effector moiety is delivered and binds to TCRs of tissues expressing autoantigens.

TCR multimers provided herein can be contemplated for clinical use in essentially all situations in which TCR engineered T cells are used, or in some embodiments, in adoptive T cell transfer. Distinct from TCR engineered cells or adoptive therapies, TCR multimers of the present disclosure provide an advantage of not requiring isolation and expansion of patient T cells. Furthermore, such TCR multimers can be pre-prepared and stored such that they are shelf-ready for use.

In some embodiments, a TCR multimer is administered to a subject with cancer and modulates the immune response to the cancer in the subject. Accordingly, in certain embodiments, the method comprises administering to the subject a TCR multimer of the disclosure wherein the TCR moiety of the TCR multimer recognizes a cancer antigen of the subject's cancer. In some embodiments, the cancer is a hematological cancer. In other embodiments, the cancer is a solid tumor.

In some embodiments, a TCR multimer is administered to a subject having an infectious disease. The present disclosure provides technologies that, in some embodiments, can modulate an immune response to the infectious disease in the subject. Accordingly, in certain embodiments, the method comprises administering to the subject a TCR multimer of the disclosure wherein the TCR moiety of the TCR multimer recognizes a pathogen antigen of the subject's infectious disease. In some embodiments, the infectious disease is a viral infection.

In other embodiments, a TCR multimer is administered to a subject undergoing vaccination and modulates the immune response to the vaccine antigen in the subject. Accordingly, in some embodiments, the method comprises administering to the subject the vaccine and a TCR multimer of the disclosure, wherein the TCR moiety of the TCR multimer recognizes an antigen of the vaccine to thereby enhance an immune response to a vaccine. In some embodiments, the vaccine and the TCR multimer are administered simultaneously to the subject. In another embodiment, the vaccine is administered prior to administration of the TCR multimer. In another embodiment, the TCR multimer is administered prior to the administration of the vaccine.

### D. Diseases

The present disclosure provides the insight that one or more immune-mediated disease, such as autoimmune disease, inflammatory disease, and/or cancer, may be detected, diagnosed, and/or treated using a composition comprising a TCR multimer as provided herein.

In some embodiments, the cancer is or comprises a solid tumor. In some embodiments, the cancer is a hematological cancer.

For example, in certain embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, vascularized tumor, pituitary tumor, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor, T cell leukemia, or non-Hodgkin's lymphoma (such as a B-cell lymphoma or a T-cell lymphoma). In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell s T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

In certain embodiments, a TCR multimer of the disclosure is administered to a subject with an infectious disease. In some embodiments, the infectious disease is viral. For example, in certain embodiments, the infectious disease is chronic HIV infection, Cytomegalovirus (CMV) infection, SARS-CoV19 infection, Epstein Barr Virus (EBV) infection, Human papillomavirus (HPV) infection, Hepatitis B virus (HBV) infection, or adenovirus infection. Accordingly, in some embodiments, the method comprises administering to the subject a TCR multimer of the disclosure wherein the TCR moiety of the multimer is recognized by immune cells of the subject and, in some embodiments, can act either in cis or trans to directly bind to a target cell, or to recruit another population of cells to impact a target cell population and treat the infection.

In some embodiments, a TCR multimer of the disclosure is administered to a subject with an autoimmune disease or disorder to downmodulate the autoimmune response in the subject. Accordingly, in some such embodiments, the method comprises administering to the subject a TCR multimer of the disclosure wherein the TCR moiety of the multimer is recognized by immune cells of the subject and, in some embodiments, can act either in cis or trans to directly bind to a target cell, or to recruit another population of cells to impact a target cell population.

In various embodiments, the autoimmune disease is one of the following (with known disease-associated HLA alleles shown in parentheses): Type I diabetes (HLA-DQ2, HLA-DQ8), celiac disease (HLA-DQ2, HLA-DQ8), primary sclerosing cholangitis (HLA-DR8, HLA-DRB1), vitiligo and autoimmune skin diseases (HLA-B27), atopic dermatitis (HLA-DRB1), rheumatoid arthritis, psoriatic arthritis, and ulcerative colitis, ankylosing spondylitis, Crohn's disease, inflammatory bowel disease, Alzheimer's disease, uveitis, psoriasis, scleroderma, lupus erythematosus, Parkinson's disease, amyotrophic lateral sclerosis, autoimmune hepatitis, primary biliary cholangitis, Sjogren's syndrome, narcolepsy, multiple sclerosis, alopecia areata, Grave's disease, Addison's disease, Hashimoto's disease, Myasthenia gravis, neuromyelitis optica, pemphigus vulgaris, bullous pemphigoid, myelin oligodendrocyte glycoprotein antibody-associated disease and acquired factor VIII deficiency. In certain embodiments, the autoimmune disease is Type I diabetes. In some embodiments, the peptide used in the TCR multimer is a Type I diabetes-associated peptide, such as an insulin peptide.

### EXAMPLES

Below are examples of specific embodiments for carrying out what is disclosed herein. The examples are offered for illustrative purposes only and are not intended to limit scope.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, PROTEINS: STRUCTURES AND MOLECULAR PROPERTIES (W.H. Freeman and Company, 1993); A. L. Lehninger, BIOCHEMISTRY (Worth Publishers, Inc., current addition); Sambrook et al. MOLECULAR CLONING: A LABORATORY MANUAL (2nd Edition, 1989); METHODS IN ENZYMOLOGY (S. Colowick and N. Kaplan eds., Academic Press, Inc.); REMINGTON'S PHARMACEUTICAL SCIENCES, 18th Edition (Easton, Pennsylvania: Mack Publishing Company, 1990); Carey and Sundberg ADVANCED ORGANIC CHEMISTRY 3^{rd} Ed. (Plenum Press) Vols A and B (1992).

Unless otherwise stated, all reagents and chemicals were obtained from commercial sources and used without further purification.

### Example 1—Recombinant Expression of TCR Multimers

This example describes the expression, purification and analysis of an exemplary TCR-IgM/J-chain multimer.

D046C1 is a TCR specific for the peptide NLVPMVATV (SEQ ID NO: 7) bound to MHC I HLA-A2. Constructs with mutations in the α and β regions were used to reduce glycosylation and to stabilize the TCR α-β association with an additional disulfide. Exemplary glycosyation mutations are: TRAV: N22Q; TRAC: N90Q, N109Q; TRBV: N86Q; TRBC: N85.6Q, using IMGT numbering). Exemplary additional cysteine mutations to stabilize the TCR α-β association with an additional disulfide are: TRAC: T84C; TRBC: S79C, C85.1A). To further promote disulfide bond formation, the first three residues of the TRAC connecting region (amino acids ESSC, E202-C205 with sequential numbering) and the first two residues of the TRBC connection region (amino acids DC, D246-C247) were included at the C-terminus of the α constant region and β constant region, respectively.

Exemplary glycosyation mutations in D046C1α and D046C1 β are shown in TABLE 1 and TABLE 2 respectively. TABLE 1 shows exemplary glycosyation mutations in D046C1 α and TABLE 2 shows exemplary glycosyation mutations in D046C1 β.

TABLE 1
Mutations in D046C1 α used for SEQ ID NO: 69

| IMGT numbering | Sequential Numbering | Region |
|---|---|---|
| N22Q | N22Q | TRAV |
| T84C | T158C | TRAC |
| N90Q | N177Q | TRAC |
| N109Q | N188Q | TRAC |

TABLE 2
Mutations in D046C1 β used for SEQ ID NO: 14

| IMGT numbering | Sequential Numbering | Region |
|---|---|---|
| N86Q | N74Q | TRBV |
| S79C | S173C | TRBC |
| N85.5Q | N186Q | TRBC |
| C85.1A | C191A | TRBC |

Plasmids encoding TCR D046C1 α chain-IgM (SEQ ID NO: 69), TCR D046C1 β (SEQ ID NO: 14) and J-chain (SEQ ID NO: 70) were transiently transfected into ExpiCHO cells using Expifectamine to express the TCR-IgM multimer, D046C1-IgM. D046C1 α (SEQ ID NO: 69) included the following amino acids ESSC (E202-C205 with sequential numbering), corresponding to the first three residues of TRAC connecting region. D046C1 β (SEQ ID NO: 14) included the following amino acids DC (D246-C247), corresponding to the first two residues of the TRBC connection region.

Cells were cultured for 11 days and harvested by centrifugation to separate the supernatant from the cells. Samples from the cell supernatant were subjected to SDS-PAGE and further analyzed by western blotting. The samples were split and analyzed under non-reducing/non-boiled conditions with NuPAGE™ Tris-Acetate 3-8% Gels (ThermoFisher) at 4° C. and reduced/boiled conditions with Bolt 4-12% Bis-Tris Plus Gels at room temperature. After the SDS-PAGE separation, western blots were probed with Dylight800-labeled anti-human IgM (ThermoFisher) and Dylight680-labeled anti-Flag (ThermoFisher) to assess expression levels. D046C1-IgM was purified by immobilized metal affinity chromatography (IMAC) using Nickel Sepharose excel resin (Cytiva) utilizing the His tag on the J-chain. The Ni Excel resin was pre-equilibrated in binding buffer (23 mM sodium phosphate, 500 mM NaCl, pH 7.4). Supernatant containing protein of interest was incubated with resin overnight and gravity flow purification was performed the next day. The Ni Excel resin was washed with wash buffer (23 mM sodium phosphate, 500 mM NaCl, (20 mM imidazole, pH 7.4) to remove unbound protein contaminants. Protein of interest was eluted using a gradient of 100 mM-500 mM imidazole in 23 mM sodium phosphate, 500 mM NaCl, pH 7.4. IMAC elution fractions were analyzed by western blot and fractions corresponding to intact complex of D046C1-IgM were identified. IMAC elution fractions were pooled based on total amount and purity of D046C1-IgM determined by western blot, and concentrated using ultrafiltration. The concentrated D046C1-IgM was loaded onto a HiLoad 16/600 Superdex 200 pg size exclusion chromatography (SEC) column pre-equilibrated with SEC buffer (20 mM HEPES, 150 mM NaCl, pH 7.2) for further purification. Elution fractions from the SEC were pooled based on total amount and purity of D046C1-IgM determined by western blot, and concentrated using ultrafiltration.

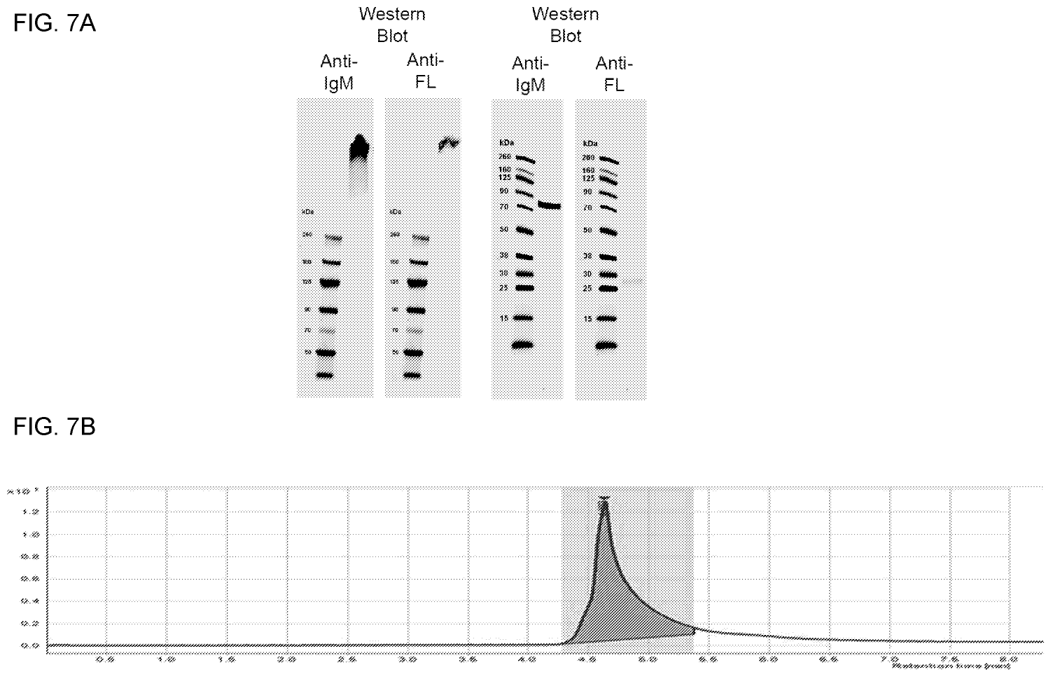

Samples were characterized by SDS-PAGE, western blot, and analytical size exclusion chromatography (SEC). Western blot of the non-reduced sample shows co-migrating high molecular weight bands (migrating higher than the highest molecular weight marker at 260 kDa) on blots probed with anti-IgM antibody and anti-Flag antibody, indicating the formation of an intact complex of TCR-IgM/J-chain. The anti-IgM western blot of the reduced sample shows a band at approximately 72 kDa, corresponding to the TCR-α-IgM. The anti-Flag western blot of the reduced sample shows a band at approximately 28 kDa corresponding to the J-chain (FIG. 7A, for D046C1-IgM). The calculated molecular weight of D046C1-α-IgM is 60.7 kDa and J-chain is 18.3 kDa. Size differences on the SDS-PAGE from observed and calculated molecular weight are believed to result from the presence of glycosylation on both proteins. Analytical size exclusion chromatography was performed to assess homogeneity and size using an AdvanceBio SEC 300 column, 4.6×50 mm, 2.7 μm (Agilent). FIG. 7B shows the analytical SEC for D046C1-IgM, where the chromatogram shows a single peak indicative of a homogeneous sample with an apparent size of greater than 670 kDa (greater than the molecular weight of the largest protein in the gel filtration standard). This demonstrates that TCR-IgM/J-chain complexes can assemble into multimers.

### Example 2—Binding of TCR-IgM to pMHC

This example describes the analysis of binding of exemplary TCR-IgM to pMHC by Biolayer Interferometry.

D046C1-IgM, containing the TCR specific for NLVPMVATV-HLA-A*02-01, was assessed for binding to biotinylated NLV/HLA-A2 by Biolayer Interferometry (Octet, Sartorius). D046C1-IgM was produced essentially as described in Example 1 using plasmids encoding: TCR D046C1 α chain-IgM (SEQ ID NO: 69), TCR D046C1 β (SEQ ID NO: 14) and J-chain (SEQ ID NO: 70). A bivalent version of D046C1 TCR (D046C1-IgG) was assessed for comparison to D046C1-IgM.

D046C1-IgG was prepared by standard methods. Briefly, to produce D046C1-IgG, plasmids encoding TCR D046C1 α chain-IgG (SEQ ID NO: 71), TCR D046C1 β (SEQ ID NO: 14) were transiently transfected into ExpiCHO cells using Expifectamine. Cells were cultured for 11 days and harvested by centrifugation to separate the supernatant from the cells. D046C1-IgG was purified by ProteinA purification using standard methods before exchange into 20 mM HEPES, 150 mM NaCl, pH 7.2. D046C1-IgG homogeneity and purity was confirmed by SDS-PAGE and SE-HPLC.

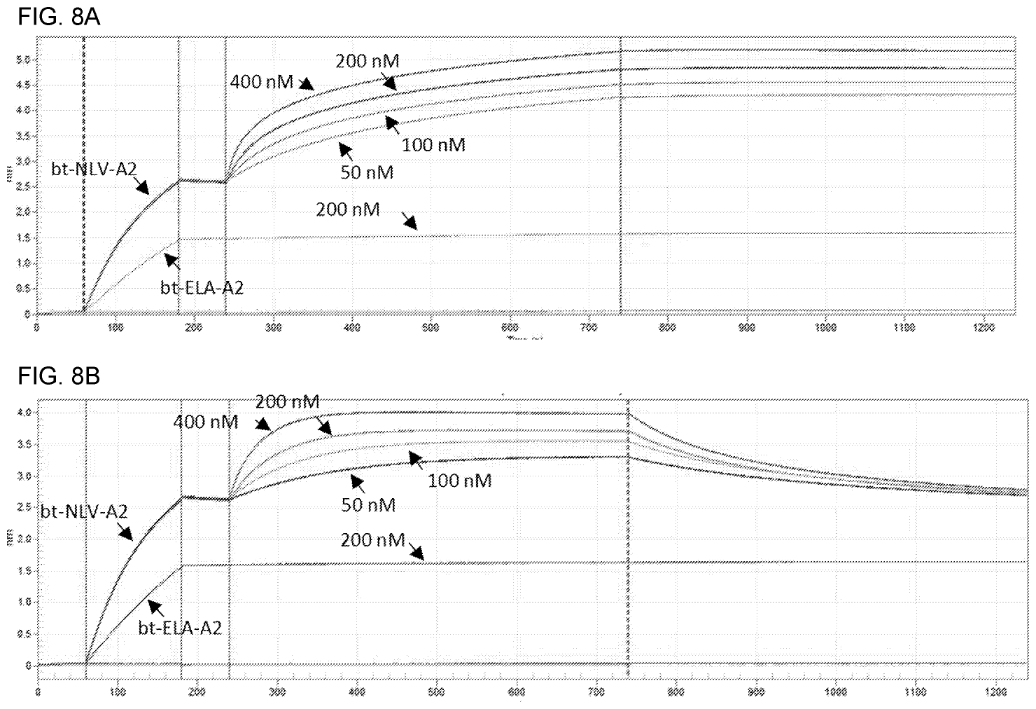

NLVPMVATV peptide-loaded HLA-A2 monomer (NLV peptide-SEQ ID NO: 7, b2m-SEQ ID NO: 76, HLA-A2 SEQ ID NO: 77) was prepared by refolding and subsequently biotinylated using a BirA biotinylation kit (Avidity), the final product is referred to as bt-NLV-A2 monomer. The same process was followed using ELAGIGILTV (ELA) peptide (SEQ ID NO: 72), the final product is referred to as bt-ELA-A2 monomer, bt-NLV-A2 monomer or bt-ELA-A2 monomer was loaded onto streptavidin coated tips as ligand. Monomer-loaded ligands (loaded onto the streptavidin coated tips) were then combined with D046C1-IgM (FIG. 8A) or D046C1-IgG (FIG. 8B) in varying concentrations (50 nM, 100 nM, 200 nM, and 400 nM) and the association rates were measured. After association of D046C1-IgM or D046C1-IgG analytes dissociation rates were measured by combination with kinetics buffer (PBS, 0.05% Sodium Azide, 0.02% Tween-20, and 0.1% BSA, at pH 7.4; i.e., by introducing the tips into the buffer). Neither TCR-IgG nor TCR-IgM showed non-specific binding to ELA-A2. The dissociation interferogram for D046C1-IgM indicates a very slow dissociation. The estimated KD of D046C1-IgG for bt-NLV-A2 is in the nanomolar range, while the estimated KD of D046C1-IgM for bt-NLV-A2 is in the picomolar range. The slower apparent dissociation and stronger apparent affinity (lower KD) support a higher avidity effect of the D046C1-IgM in binding to its target.

### Example 3—Preparation of J Chain-Effector Moiety Constructs

This Example describes the expression, purification, and analysis of TCR-IgM with different effector moieties.

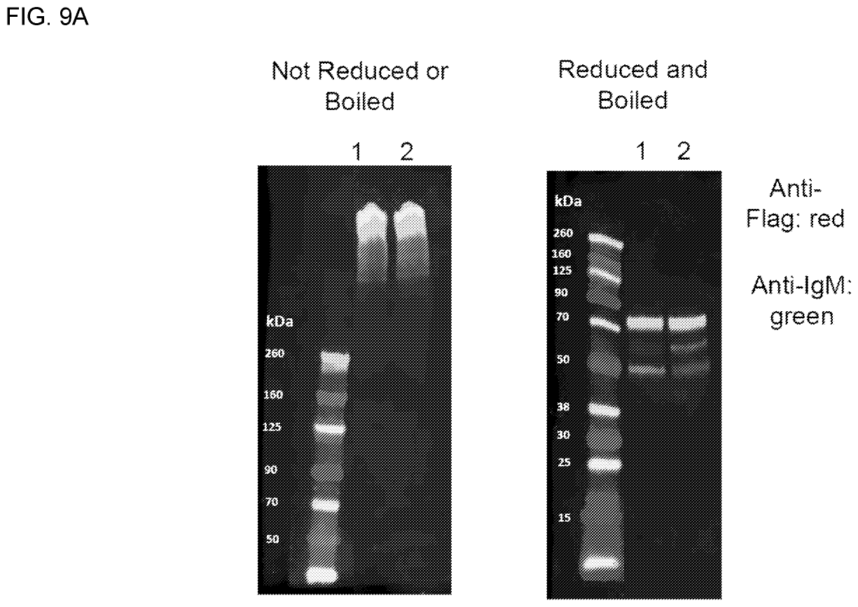

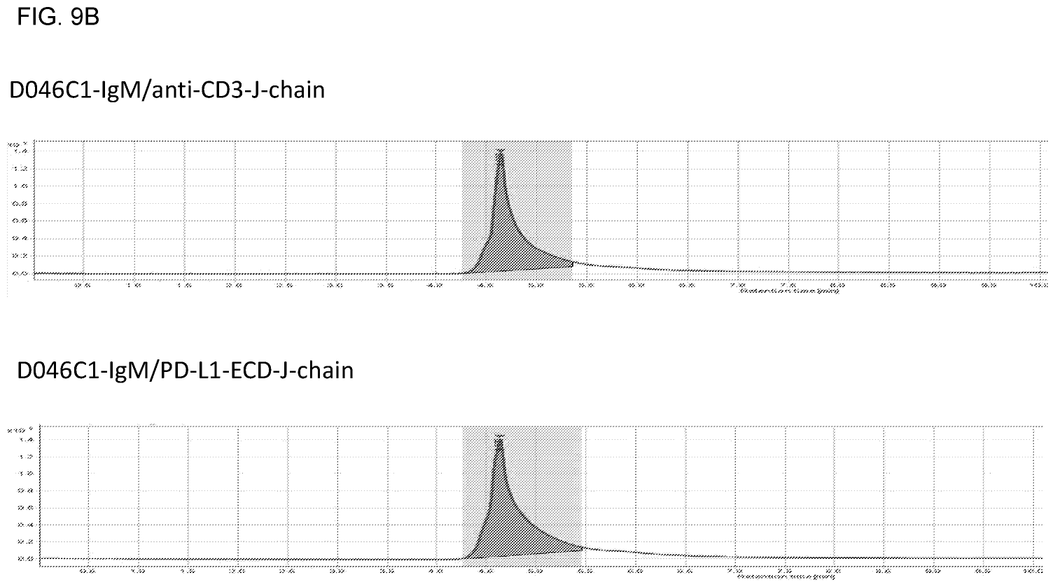

Expression, purification, and characterization of TCR-IgM with anti-CD3 and PD-L1 effector moieties was demonstrated. Plasmids encoding TCR D046C1 α chain-IgM (SEQ ID NO: 69), TCR D046C1 β (SEQ ID NO: 14), and anti-CD3-scFv-J-chain-Flag-His (SEQ ID NO: 73 or PD-L1-ECD-J-chain-Flag-His (SEQ ID NO: 74 were used for transfection of mammalian cells. TCR-IgM-ED proteins were expressed, purified, and analyzed essentially as described in Example 1. The proteins were characterized by SDS-PAGE, western blot, and analytical size exclusion chromatography. Probing of western blots run under non-reducing/non-boiling conditions simultaneously with anti-Flag and anti-IgM antibody showed a band at very high molecular weight (migrating higher than the highest molecular weight marker at 260 kDa) for both proteins (FIG. 9A, left panel, lane 1 corresponds to D046C1-IgM/anti-CD3-J-chain, lane 2 corresponds to D046C1-IgM/PD-L1-ECD-J-chain). For both proteins, the anti-IgM and anti-Flag antibodies showed a band in the same position on the blot, indicating co-migration of TCR-IgM and Flag-containing species and, therefore, the formation of an intact complex of D046C1-IgM/anti-CD3-J-chain and D046C1-IgM/PD-L1-ECD-J-chain.

The anti-IgM western blot of the reduced samples of D046C1-IgM/anti-CD3-J-chain and D046C1-IgM/PD-L1-ECD-J-chain shows a band at approximately 72 kDa, corresponding to the TCR-α-IgM.

The anti-Flag western blot of the D046C1-IgM/anti-CD3-J-chain reduced sample shows a band at approximately 50 kDa corresponding to the anti-CD3-J-chain (FIG. 9A, right panel, lane 1). The calculated molecular weight of anti-CD3-J-chain is 45.1 kDa. It is believed that the differences in western blot-observed and calculated molecular weight are likely due to glycosylation.

The anti-Flag western blot of the D046C1-IgM/PD-L1-ECD-J-chain reduced sample shows a band at approximately 60 kDa corresponding to the PD-L1-ECD-J-chain (FIG. 9A, right panel, lane 2). The calculated molecular weight of PD-L1-ECD-J-chain is 45.1 kDa. The differences in western blot-observed and calculated molecular weight are likely due to glycosylation.

Analytical size exclusion chromatography was performed to assess homogeneity and size using an AdvanceBio SEC 300 column, 4.6×50 mm, 2.7 μm (Agilent). FIG. 9B shows the analytical SEC for D046C1-IgM/anti-CD3-J-chain (upper panel) and D046C1-IgM/PD-L1-ECD-J-chain (lower panel). Each chromatogram shows a single peak indicative of a homogeneous sample. This demonstrates that D046C1-IgM/anti-CD3-J-chain and D046C1-IgM/PD-L1-ECD-J-chain can assemble into multimers of the expected size.

### Example 4—Inhibition of Effector T Cells In Vitro by TCR-Ig Multimers Coupled to an Immunosuppressive Agent

This example describes the inhibition of effector T cells in vitro by TCR-Ig multimers coupled to an immunosuppressive agent.

TCR-Ig multimers coupled to an immunosuppressive agent like TGF-β, IL-10, HLA-G, an LILRB1 or LILRB2 agonist, or an exhaustion-inducing agent like PD-L1, or a PD1, CTLA-4, Lag3 or TIM3 agonist, can be assessed for their ability to reduce TCR signaling, proliferation, cytokine secretion, or cell killing by auto-reactive T cells, as shown in FIG. 6.

To assess TCR signaling inhibition, J76-CD8-NFAT-GFP cell lines expressing recombinant TCRs specific to an auto-antigen (or T cells expanded from PBMCs with an auto-antigen peptide) are incubated with an allele-matched APC presenting autoantigen-MHC complexes, with or without the TCR-Ig-immunosuppressor multimers. It is not required that the T cells and the TCR-Ig-immunosuppressor multimers recognize the same autoantigen peptide-MHC, as long as the APC cell line presents peptides recognized by each in the context of the relevant MHC allele. Targeting of the immunosuppressor to the APCs results in immunosuppression of autoreactive T cells. Analysis of NFAT-GFP expression over time at 37° C. will show a reduction in NFAT signaling in the presence of the TCR-Ig multimers. Antigen-specific T cells co-cultured with allele-matched APCs loaded with the cognate peptide can also be stained with anti-CD69-APC (Clone FN50) antibodies and analyzed by flow cytometry to measure a reduction in the percentage of CD69+ cells in the presence of the TCR-Ig multimers. A third possible measure of effects on TCR signaling is to monitor inhibition of phosphorylation of SLP-76, PLCγ, and ZAP-70 by western blot when TCR-Ig multimers are incubated with antigen-specific T cells co-cultured with APCs loaded with cognate peptide.

To assess inhibition of T cell proliferation by TCR-Ig-immunosuppressor multimers, autoreactive T cells are loaded with a dye like CFSE, then co-cultured with allele-matched APCs loaded with autoantigen peptides plus and minus the multimers. Every generation of cells appears as a new lower mean fluorescent intensity (MFI) peak on a flow cytometry histogram. Reduced proliferation in the presence of the TCR-Ig multimers is evidenced by a shift to a higher percentage of T cells with high MFI.

To assess inhibition of cytokine secretion by TCR-Ig-immunosuppressor multimers, multimers are added to co-cultures of auto-reactive T cells and allele-matched APCs loaded with cognate auto-antigen peptides, and the culture is monitored for IL-2, IFN-γ or TNFα by ELISA, ELISPOT, or Mesoscale, or a combination thereof. A reduction in secretion indicates immunosuppression by the TCR-Ig multimer.

Inhibition of T cell cytotoxicity by TCR-Ig-immunosuppressor multimers is measured by coculturing auto-reactive T cells with target allele-matched APCs loaded with the cognate auto-antigen peptides. By differential labeling of the T cells versus the target APCs, the loss of viability (e.g., assessed by live/dead stain) and overall reduction of APC cell counts can be monitored by flow cytometry. Co-incubation of TCR-Ig-immunosuppressor multimers inhibits targeted killing of APCs, resulting in higher numbers of live cells.

### Example 5—Specific Deletion of APC or Tumor Cells In Vitro by T Cell Redirection with TCR-Ig Multimers Coupled to Anti-CD3

Allele-matched APCs loaded with cognate peptide (or tumor cell lines presenting the cognate peptide in the relevant MHC allele) are co-cultured with CD8 T cells and TCR-Ig multimers coupled to anti-CD3 (FIG. 5). TCR multimers are targeted to the APC/tumor cell lines, and the anti-CD3 domain recruits effector T cells, which kill the antigen-specific T cell via secretion of perforin and granzyme. Differential labeling of the different cells (e.g., by CFSE or, for instance, a discriminating staining antibody) allows determination of the relative number of each cell type by flow cytometry, and the loss of the antigen-presenting cells caused by the TCR-Ig-anti-CD3 multimers can be assessed.

### Example 6—Recombinant Expression of TCR Multimers Targeting Gp100Pep-HLA-A*02:01 Complex

Expression and purification was additionally demonstrated with TCR-IgM's targeting gp100pep-HLA-A*02:01 complex, where gp100pep is the peptide YLEPGPVTA (SEQ ID NO: 9798) derived from human glycoprotein 100, which is significantly overexpressed in melanoma cancer cells. To generate TCR-IgMCu3-4 variants, the TCR alpha chain was fused to the IgG1-hinge followed by IgMCu3-4 to generate the corresponding plasmid. Plasmids encoding polypeptides according to TABLE 3 and TABLE 4 were transiently transfected into Expi293F cells (ThermoFisher) using Expifectamine to express the corresponding TCR-IgM/Immunomodulator-J-chain.

TABLE 3
Construct Table for TCR-IgMs

| | | | TCR-alpha | Immunomodulator- |
|---|---|---|---|---|
| | Protein | TCR-beta | chain-IgM | J-chain polypeptide |
| Protein Name | ID | polypeptide ID | polypeptide ID | ID |
| Par-gp-IgM₂₋₄*/Jch- | PL436 | PS1503 | PS1465 | PS848 |
| OKT3 | | (SEQ ID NO: 89) | (SEQ ID NO: 88) | (SEQ ID NO: 17) |
| AM-gp-IgM₂₋₄/OKT3- | PL433 | PS1421 | PS1416 | PS1083 |
| Jch | | (SEQ ID NO: 86) | (SEQ ID NO: 87) | (SEQ ID NO: 73) |
| Par-gp-IgM₂₋₄/OKT3- | PL435 | PS1503 | PS1465 | PS1083 |
| Jch | | (SEQ ID NO: 89) | (SEQ ID NO: 88) | (SEQ ID NO: 73) |
| AM-gp-IgM₂₋₄/Jch- | PL434 | PS1421 | PS1416 | PS848 |
| OKT3 | | (SEQ ID NO: 86) | (SEQ ID NO: 87) | (SEQ ID NO: 17) |
| Par-gp-IgM₂₋₄/U1v9- | PL421 | PS1503 | PS1465 | PS1429 |
| J | | (SEQ ID NO: 89) | (SEQ ID NO: 88) | (SEQ ID NO: 96) |
| AM-gp-IgM₂₋₄/U1v9- | PL420 | PS1421 | PS1416 | PS1429 |
| J | | (SEQ ID NO: 86) | (SEQ ID NO: 87) | (SEQ ID NO: 96) |
| Par-gp-IgM₃₋₄/Jch- | PL456 | PS1503 | PS1638 | PS848 |
| OKT3 | | (SEQ ID NO: 89) | (SEQ ID NO: 90) | (SEQ ID NO: 17) |
| Par-gp-IgM₃₋₄/OKT3- | PL465 | PS1503 | PS1638 | PS1083 |
| Jch | | (SEQ ID NO: 89) | (SEQ ID NO: 90) | (SEQ ID NO: 73) |
| AM-gp-IgM₃₋₄/Jch- | PL466 | PS1421 | PS1632 | PS848 |
| OKT3 | | (SEQ ID NO: 86) | (SEQ ID NO: 91) | (SEQ ID NO: 17) |

*IgM₂₋₄ is used interchangeably herein with IgMCu₂₋₄ (SEQ ID NOs: 78-83).
**IgM₃₋₄ variants herein used g1Hn-IgMCu3-4 (SEQ ID NO: 95)

TABLE 4
Construct Table for Non-IgM proteins

| Protein Name | Protein ID | TCR-beta chain ID | TCR-alpha chain ID |
|---|---|---|---|
| U1V9-AM-gp | PL373 | PS1364 | PS1365 |
| | | (SEQ ID NO: 92) | (SEQ ID NO: 93) |
| Par-gp-U1v9 | PL427 | PS1503 | PS1463 |
| | | (SEQ ID NO: 89) | (SEQ ID NO: 94) |

After 5 days supernatant was recovered, then purification and analysis was performed according to Example 1.

Proteins were analyzed by SDS-PAGE, Western Blotting and SE-HPLC according to Example 1 and Example 3. The reduced SDS-PAGE gel shows: a higher molecular weight band corresponding to the TCR-alpha chain fused to IgM-Cu2-4 (approx. 90 kDa) or TCR-alpha chain fused to IgMCu3-4 (approx. 70 kDa), a band at 55 kDa corresponding to the Immunomodulator-J-chain or J-chain-Immunomodulator fusion, and a band migrating at approx. 35 kDa corresponding to the TCR-beta chain. The Western blot shows a diffuse band stained with the anti-IgM antibody, corresponding to the TCR-alpha chain fused to IgM-Cu2-4 (approx. 90 kDa) or TCR-alpha chain fused to IgMCu3-4 (approx. 70 kDa), and a band detected with the anti-Flag antibody at approx. 55 kDa corresponding to the Immunomodulator-J-chain or J-chain-Immunomodulator fusion. SDS-PAGE and Western blot results indicate that polypeptide chains of the expected size and containing IgM or Flag-tag are present in the purified TCR-IgM/Immunomodulator-J-chain samples. Differences from the theoretical and observed molecular weights is likely due to glycosylation.

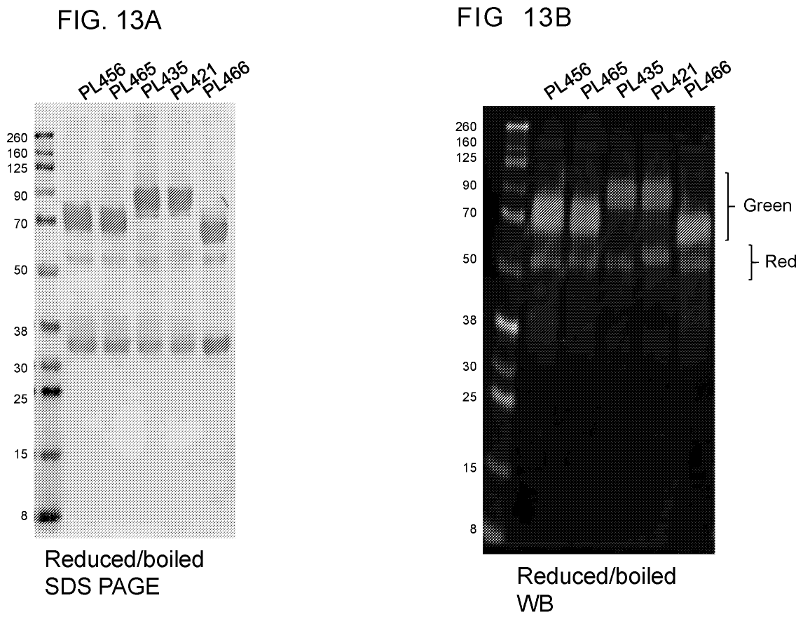

FIGS. 13A and 13B show that TCR-IgM3-4 used for PL456 and PL465 can be expressed and purified stably.

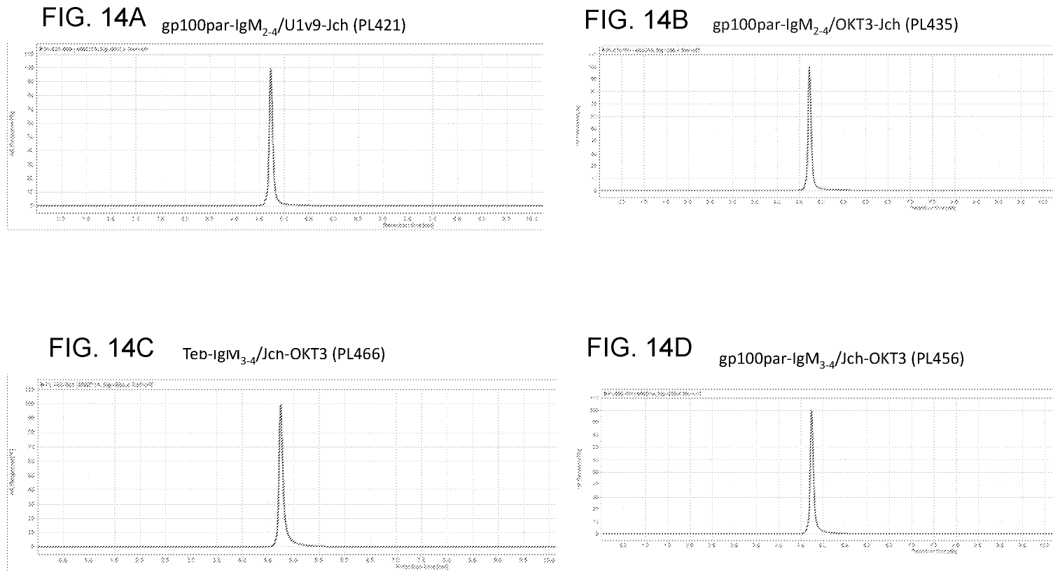

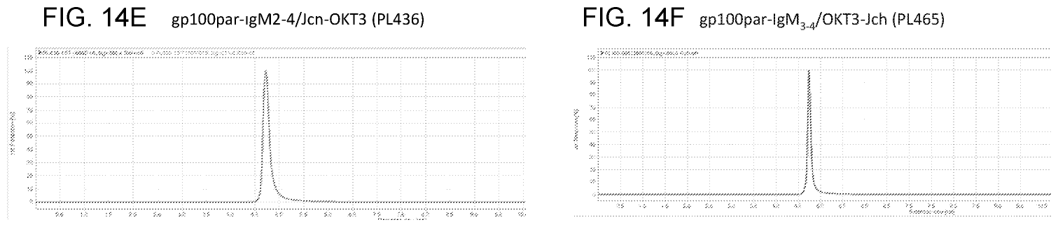

Analytical size exclusion chromatography was performed as described in Example 1. FIGS. 14A to 14F show the analytical SEC for purified PL421, PL466, PL436, PL435, PL456, PL465 proteins, respectively. The chromatograms show a single peak indicative of a homogeneous sample with an apparent size of greater than 670 kDa (greater than the molecular weight of the largest protein in the gel filtration standard). This demonstrates that TCR-IgM/J-chain complexes can assemble into multimers.

### Example 7—Comparison of Binding of TCR-IgM to Monovalent TCR

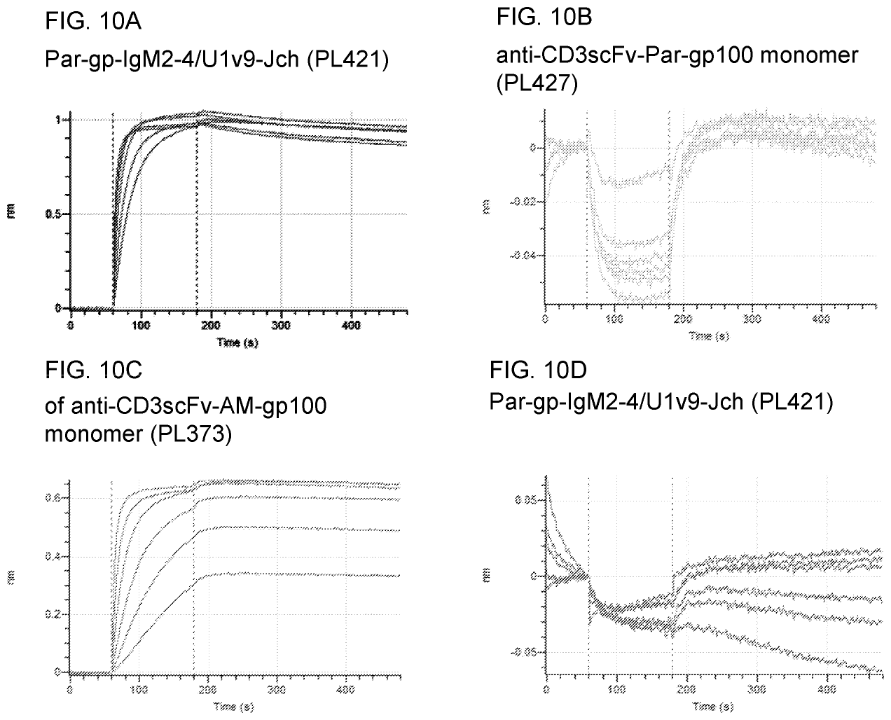

TCR-IgM proteins were expressed and purified according to Example 6. TCR-IgM proteins were tested for binding to pMHC by Biolayer Interferometry using Octet. Biotinylated YLEPGPVTA-HLA-A*02:01 complex (MPL141) or an irrelevant-peptide-HLA-A*02:01 complex (MPL138) was loaded onto Streptavidin tips at 1.25 μg/mL. pMHC-loaded tips were then combined with: Par-gp-IgM-Cu2-4/U1v9-J (PL421), anti-CD3scFv-AM-gp100 monomer (PL373) or anti-CD3scFv-Par-gp100 monomer (PL427) at different concentrations (800-25 nM with 2-fold dilutions). PL421 and PL427 are both derived from a non-affinity matured TCR, and contain the same TCR variable region sequences. The TCR region sequence of PL373 is an affinity matured version of the TCR for PL427. Association was monitored for 120 s and dissociation was monitored for 300 s. Results were analyzed by Octet Data Analysis HT 10.0.3.12 software (Sartorius) using a 1:1 global fit model. Data from the Octet binding assays are shown in FIGS. 10A-10D and are summarized in TABLE 5.

TABLE 5
Binding to gp100/HLA-A*02:01 (MPL141)

| Protein ID | KD (M) | ka (1/Ms) | kdis (1/s) |
|---|---|---|---|
| PL421 | 1.02E−10 | 1.22E+06 | 1.25E−04 |
| PL427 | Not calculated | | |
| PL373 | 3.12E−12 | 1.82E+05 | 5.68E−07 |
| PL421: irr peptide-MHC | Not calculated | | |
| (MPL138) | | | |

Anti-CD3scFv-Par-gp100 monomer (PL427) shows no apparent association in the concentration range tested, whereas anti-CD3scFv-AM-gp100 monomer (PL373) shows strong association and slow dissociation with low picomolar affinity. Par-gp-IgM-Cu2-4/U1v9-J (PL421) shows strong association and slow dissociation and binds with approximately 100 pM apparent affinity (avidity). No binding to irrel-pep-HLA-A*02:01 complex was observed for Par-gp-IgM-Cu2-4/U1v9-J (PL421). These results demonstrate that fusion of a TCR-Fc enables high apparent affinity (avidity) to cognate pMHC.

### Example 8—Binding of Gp100-TCR-IgM Variants to pMHC

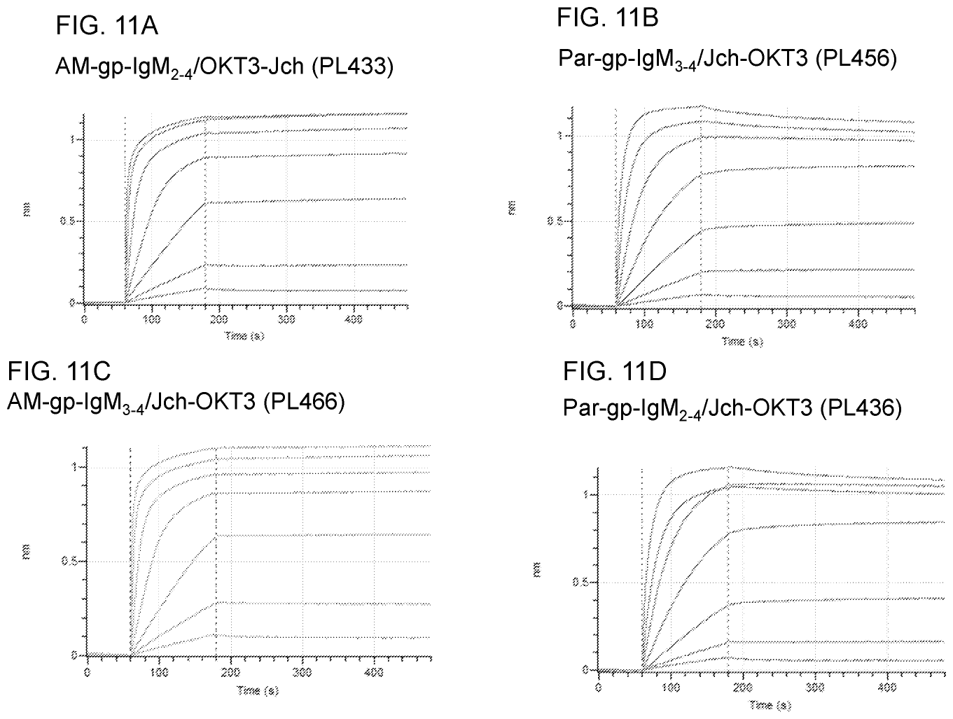

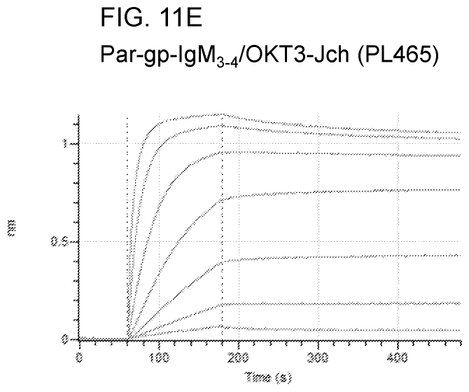

TCR-IgM proteins were expressed and purified according to Example 6. TCR-IgM proteins containing different IgM-Fc lengths (Cu2-4 and Cu3-4) and different immunomodulator identity and orientations were tested for binding to pMHC by Biolayer Interferometry using Octet. Biotinylated YLEPGPVTA-HLA-A*02:01 complex (MPL141) was loaded onto Streptavidin tips at 1.25 μg/mL. pMHC-loaded Streptavidin tips were then combined with: PL433, PL436, PL456, PL465 or PL466 at different concentrations (200-0.27 nM with 3-fold dilutions). Association was monitored for 120 s and dissociation was monitored for 300 s. Data from the Octet binding assays are shown in FIGS. 11A-11E and are summarized in TABLE 6.

TABLE 6
Binding to gp100/HLA-A*02:01 (MPL141)

| Protein ID | KD (M) | ka (1/Ms) | kdis (1/s) |
|---|---|---|---|
| PL433 | <1.0E−12 | 2.23E+06 | 5.10E−07 |
| PL436 | <1.0E−12 | 1.10E+06 | 6.47E−07 |
| PL456 | <1.0E−12 | 1.28E+06 | 4.44E−07 |
| PL465 | <1.0E−12 | 1.26E+06 | 6.98E−07 |
| PL466 | <1.0E−12 | 3.17E+06 | 6.26E−07 |

While Parent TCR-IgM2-4/Jch-OKT3 (PL436) had slightly faster dissociation at the higher concentrations compared to affinity matured TCR-IgMCu2-4/OKT3-Jch (PL433), the affinity matured TCR-IgMCu2-4/OKT3-Jch (PL433) and parent TCR-IgMCu2-4/Jch-OKT3 (PL436) had comparable association and dissociation profiles with apparent affinity (avidity) less than 1 pM. This demonstrates that non-affinity matured-gp-IgMCu2-4/Jch-OKT3 is able to bind with high avidity to its target pMHC.

Parent TCR IgMCu3-4 variants with different orientations of OKT3 scFv (PL456, PL465) showed pMHC-binding profiles that were comparable to affinity-matured TCR-IgMCu3-4 (PL466) with apparent affinities less than 1 pM. PL456 and PL465 showed slightly faster dissociation at the highest concentrations compared to Affinity matured-IgMCu3-4 (PL466).

This demonstrates that non-affinity matured TCR-IgMCu3-4 can bind with high avidity, comparable to affinity-matured TCR-IgMCu3-4. This additionally demonstrates that TCR-IgMCu2-4 and TCR-IgMCu3-4 bind with similar apparent affinity (avidity).

### Example 9—Binding of TCR-IgM/Anti-CD3 to CD3 Protein

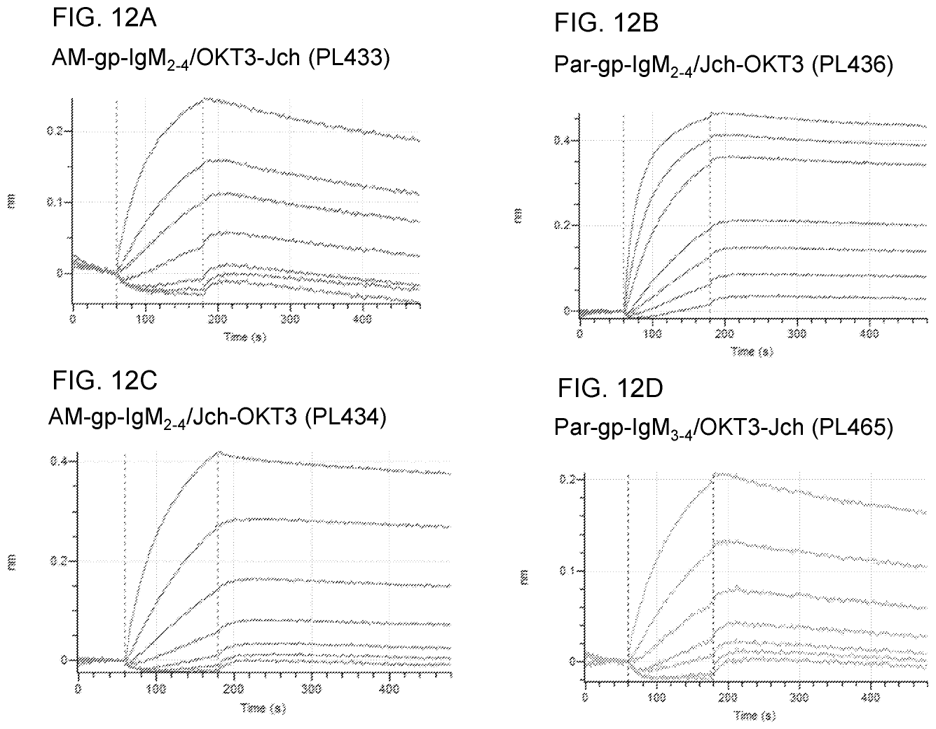

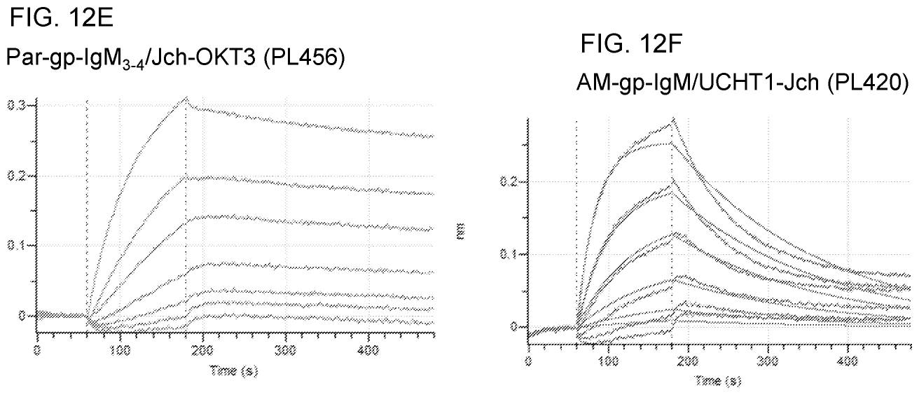

TCR-IgM containing different immunomodulator identity and orientations were tested for binding to CD3 by Biolayer Interferometry using Octet (Sartorius). Biotinylated human CD3 epsilon/gamma (ThermoFisher Scientific) was loaded onto Streptavidin tips at 1.25 μg/mL. CD3-loaded Streptavidin tips were then combined with: PL433, PL434, PL436, PL456 or PL465 at different concentrations (400-6.25 nM with 2-fold dilutions). PL420 was tested at (800-25 nM with 2-fold dilutions). Association was monitored for 120 s and dissociation was monitored for 300 s. Data from the Octet Binding Assays are shown in FIGS. 12A-12F and are summarized in TABLE 7.

TABLE 7
Binding to CD3

| Protein ID | KD (M) | ka (1/Ms) | kdis (1/s) |
|---|---|---|---|
| PL433 | 2.08E−08 | 4.85E+04 | 1.01E−03 |
| PL434 | 4.01E−09 | 4.74E+04 | 1.90E−04 |
| PL436 | 1.27E−09 | 1.08E+05 | 1.38E−04 |
| PL456 | 1.16E−08 | 4.21E+04 | 4.88E−04 |
| PL465 | 2.01E−08 | 3.38E+04 | 6.81E−04 |
| PL420 | 1.14E−07 | 4.57E+04 | 5.52E−03 |

PL436 and PL434, which have the same immunomodulator-J-chain fusion and which both contain IgMCu2-4, have similar affinities for CD3 in the low nanomolar range (1.3-4.0 nM). PL433 and PL465, which contain OKT3-Jch-chain fusion, have similar affinities for CD3 at approximately 20 nM. PL456, which has the same J-chain-OKT3 fusion as PL436 and PL434, has a lower binding affinity in the 12 nM range.

PL420, which contains UCHT1-v9 scFv at the N-terminus of the J-chain in the context of the affinity matured gp100-TCR-IgMCu2-4 has weaker binding than OKT3-containing TCR-IgM.

These results demonstrate that different anti-CD3 moieties at the N- or C-terminus of the J-chain are able to bind CD3 in the context of a TCR-IgM.

### Example 10—TCR IgM Multimer Induced Activation of CD8 T Cells in Response to Cognate Peptide Presentation

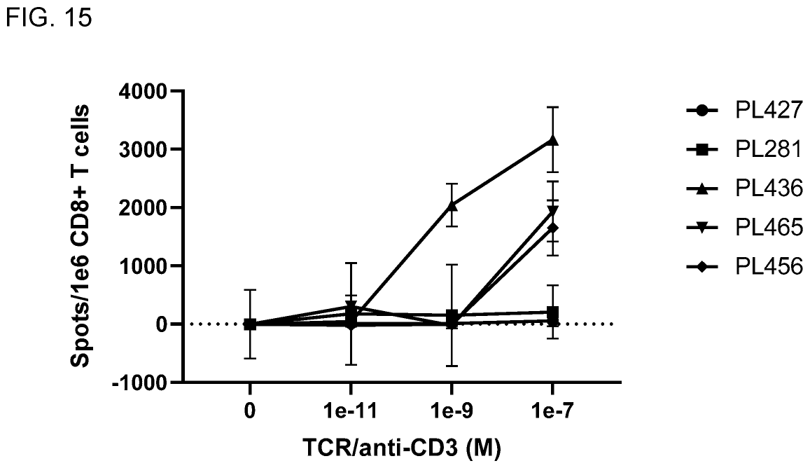

Allele-matched APCs (T2s) loaded with heteroclitic gp100 peptide YLEPGPVTV (SEQ ID NO: 106) were co-cultured with purified peripheral CD8+ T cells and TCR-IgM/anti-CD3 multimers (or TCR-bispecific). T cells and APCs were mixed in equal parts (30000 cells: 30000 cells), with TCR/anti-CD3 molecule, in 200 uL of AIM-V media (ThermoFisher) supplemented with 5% hAB serum and 1× Glutamax (ThermoFisher), in an ELISpot plate (Mabtech). The mixtures were incubated overnight for 18 hours at 37° C. with 5% CO₂. Each condition was in triplicate. T cell activation was measured using IFNγ ELISpot, which quantifies IFNγ secretion by cells. Plates were developed according to the manufacturer's instructions (Mabtech) and analyzed using an ELISpot plate reader (Mabtech) (FIG. 15). gp100par-IgM2-4/Jch-OKT3 (PL436), gp100par-IgM3-4/Jch-OKT3 (PL456), and gp100par-IgM3-4/OKT3-Jch (PL465) show a dose-dependent activation of T cells. As expected, monovalent Par-gp-U1v9 (PL427) shows no activation due to low monovalent affinity. Additionally, negative control D046C1-IgM/anti-CD3 (PL281), containing the TCR specific for a different peptide, NLVPMVATV-HLA-A*02-01 (D046C1-IgM/anti-CD3-J-chain, Example 3) did not show T cell activation. This demonstrates that TCR-IgM/anti-CD3 multimers can activate T cells towards antigen presenting cells in a peptide-specific manner.

### INCORPORATION BY REFERENCE

The entire disclosure of each of the patent and scientific documents referred to herein is incorporated by reference for all purposes.

### EQUIVALENTS

An invention may be embodied in other specific forms without departing from the spirit or essential characteristics thereof. The foregoing embodiments are therefore to be considered in all respects illustrative rather than limiting on any invention disclosed herein. Scope of an invention is thus indicated by the appended claims rather than by the foregoing description, and all changes that come within the meaning and range of equivalency of the claims are intended to be embraced therein.

SEQUENCE LISTING SUMMARY

| SEQ ID | |
|---|---|
| NO: | DESCRIPTION |
| 1 | KNEVEQSPQNLTAQEGEFITINCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKTVLDMRSMDFKSNSAVAWSNKSDFACANAFNNSIIPEDTFFPSP |
| | (TCR D046C1 alpha chain full-length wild-type sequence) |
| 2 | KNEVEQSPQNLTAQEGEFITINCSYSVGISALHWLQQHPGGGIVSLFMLSSGKKKHGRLIATINIQEK |
| | HSSLHITASHPRDSAVYICAADPSDSWGKLQFGAGTQVVVTP |
| | (TCR D046C1 alpha chain variable region wild-type sequence) |
| 3 | DIQNPDPAVYQLRDSKSSDKSVCLFTDFDSQTNVSQSKDSDVYITDKTVLDMRSMDFKSNSAVAWSNK |
| | SDFACANAFNNSIIPEDTFFPSP |
| | (TCR D046C1 alpha chain constant region wild-type sequence) |
| 4 | EPEVTQTPSHQVTQMGQEVILRCVPISNHLYFYWYRQILGQKVEFLVSFYNNEISEKSEIFDDQFSVE |
| | RPDGSNFTLKIRSTKLEDSAMYFCASSESRILTYNEQFFGPGTRLTVLEDLNKVFPPEVAVFEPSEAE |
| | ISHTQKATLVCLATGFFPDHVELSWWVNGKEVHSGVSTDPQPLKEQPALNDSRYCLSSRLRVSATFWQ |
| | NPRNHFRCQVQFYGLSENDEWTQDRAKPVTQIVSAEAWGRADC |
| | (TCR D046C1 beta chain full-length wild-type sequence) |
| 5 | EPEVTQTPSHQVTQMGQEVILRCVPISNHLYFYWYRQILGQKVEFLVSFYNNEISEKSEIFDDQFSVE |
| | RPDGSNFTLKIRSTKLEDSAMYFCASSESRILTYNEQFFGPGTRLTVL |
| | (TCR D046C1 beta chain variable region wild-type sequence) |
| 6 | EDLNKVFPPEVAVFEPSEAEISHTQKATLVCLATGFFPDHVELSWWVNGKEVHSGVSTDPQPLKEQPA |
| | LNDSRYCLSSRLRVSATFWQNPRNHFRCQVQFYGLSENDEWTQDRAKPVTQIVSAEAWGRADC |
| | (TCR D046C1 beta chain constant region wild-type sequence) |
| 7 | NLVPMVATV |
| | (HLA-A*02:01 restricted CMV pp65 epitope) |
| 8 | MAVMAPRTLVLLLSGALALTQTWAGSHSMRYFFTSVSRPGRGEPRFIAVGYVDDTQFVRFDSDAASQR |
| | MEPRAPWIEQEGPEYWDGETRKVKAHSQTHRVDLGTLRGYYNQSEAGSHTVQRMYGCDVGSDWRFLRG |
| | YHQYAYDGKDYIALKEDLRSWTAADMAAQTTKHKWEAAHVAEQLRAYLEGTCVEWLRRYLENGKETLQ |
| | RTDAPKTHMTHHAVSDHEATLRCWALSFYPAEITLTWQRDGEDQTQDTELVETRPAGDGTFQKWAAVV |
| | VPSGQEQRYTCHVQHEGLPKPLTLRWEPSSQPTISIVGIIAGLVLFGAVITGAVVAAVMWRRKSSDRK |
| | GGSYSQAASSDSAQGSDVSLTACKV |
| | (HLA-A*02:01 full-length sequence) |
| 9 | GSHSMRYFFTSVSRPGRGEPRFIAVGYVDDTQFVRFDSDAASQRMEPRAPWIEQEGPEYWDGETRKVK |
| | AHSQTHRVDLGTLRGYYNQSEAGSHTVQRMYGCDVGSDWRFLRGYHQYAYDGKDYIALKEDLRSWTAA |
| | DMAAQTTKHKWEAAHVAEQLRAYLEGTCVEWLRRYLENGKETLQRTDAPKTHMTHHAVSDHEATLRCW |
| | ALSFYPAEITLTWQRDGEDQTQDTELVETRPAGDGTFQKWAAVVVPSGQEQRYTCHVQHEGLPKPLTL |
| | RWEPSS |
| | (HLA-A*02:01; soluble domain residues 25-302) |
| 10 | MSRSVALAVLALLSLSGLEAIQRTPKIQVYSRHPAENGKSNFLNCYVSGFHPSDIEVDLL |
| | KNGERIEKVEHSDLSFSKDWSFYLLYYTEFTPTEKDEYACRVNHVTLSQPKIVKWDRDM |
| | (full length human beta-2-microglobulin) |
| 11 | KNEVEQSPQNLTAQEGEFITIQCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTQVSQSKDSDVYITDKCVLDMRSMDFKSNSAVAWSQKSDFACANAFNNSIIPEDTFFPSP |
| | (TCR D046C1 alpha chain Mutant 1) |
| 12 | KNEVEQSPQNLTAQEGEFITIQCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKCVLDMRSMDFKSNSAVAWSQKSDFACANAFQNSIIPEDTFFPSP |
| | (TCR D046C1 alpha chain Mutant 2) |
| 13 | EPEVTQTPSHQVTQMGQEVILRCVPISNHLYFYWYRQILGQKVEFLVSFYNNEISEKSEIFDDQFSVE |
| | RPDGSQFTLKIRSTKLEDSAMYFCASSESRILTYNEQFFGPGTRLTVLEDLNKVFPPEVAVFEPSEAE |
| | ISHTQKATLVCLATGFFPDHVELSWWVNGKEVHSGVCTDPQPLKEQPALQDSRYCLSSRLRVSATFWQ |
| | NPRNHFRCQVQFYGLSENDEWTQDRAKPVTQIVSAEAWGRADC |
| | (TCR D046C1 beta chain Mutant 1) |
| 14 | EPEVTQTPSHQVTQMGQEVILRCVPISNHLYFYWYRQILGQKVEFLVSFYNNEISEKSEIFDDQFSVE |
| | RPDGSQFTLKIRSTKLEDSAMYFCASSESRILTYNEQFFGPGTRLTVLEDLNKVFPPEVAVFEPSEAE |
| | ISHTQKATLVCLATGFFPDHVELSWWVNGKEVHSGVCTDPQPLKEQPALQDSRYALSSRLRVSATFWQ |
| | NPRNHFRCQVQFYGLSENDEWTQDRAKPVTQIVSAEAWGRADC |
| | (TCR D046C1 beta chain Mutant 2) |
| 15 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | D |
| | (Human Immunoglobulin J chain) |
| 16 | QVQLQQSGAELARPGASVKMSCKASGYTFTRYTMHWVKQRPGQGLEWIGYINPSRGYTNYNQKFKDKA |
| | TLTTDKSSSTAYMQLSSLTSEDSAVYYCARYYDDHYSLDYWGQGTTLTVSSGGGGSGGGGSGGGGSQI |
| | VLTQSPAIMSASPGEKVTMTCSASSSVSYMNWYQQKSGTSPKRWIYDTSKLASGVPAHFRGSGSGTSY |
| | SLTISGMEAEDAATYYCQQWSSNPFTFGSGTKLEIK |
| | (Anti-CD3 scFv sequence) |
| 17 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSQVQLQQSGAELARPGASVKMSCKASGYTFTRYTMHWVKQRPGQGLEWIGYIN |
| | PSRGYTNYNQKFKDKATLTTDKSSSTAYMQLSSLTSEDSAVYYCARYYDDHYSLDYWGQGTTLTVSSG |
| | GGGSGGGGSGGGGSQIVLTQSPAIMSASPGEKVTMTCSASSSVSYMNWYQQKSGTSPKRWIYDTSKLA |
| | SGVPAHFRGSGSGTSYSLTISGMEAEDAATYYCQQWSSNPFTFGSGTKLEIKGGGGSDYKDDDDKHHH |
| | HHH |
| | (Human Ig J chain with anti-CD3 at C-terminus + tags) |
| 18 | QVQLQQSGAELARPGASVKMSCKASGYTFTRYTMHWVKQRPGQGLEWIGYINPSRGYTNYNQKFKDKA |
| | TLTTDKSSSTAYMQLSSLTSEDSAVYYCARYYDDHYSLDYWGQGTTLTVSSGGGGSGGGGSGGGGSQI |
| | VLTQSPAIMSASPGEKVTMTCSASSSVSYMNWYQQKSGTSPKRWIYDTSKLASGVPAHFRGSGSGTSY |
| | SLTISGMEAEDAATYYCQQWSSNPFTFGSGTKLEIKGGGGSGGGGSGGGGSQEDERIVLVDNKCKCAR |
| | ITSRIIRSSEDPNEDIVERNIRIIVPLNNRENISDPTSPLRTRFVYHLSDLCKKCDPTEVELDNQIVT |
| | ATQSNICDEDSATETCYTYDRNKCYTAVVPLVYGGETKMVETALTPDACYPDGGGGSDYKDDDDKHHH |
| | HHH |
| | (Human Ig J chain with anti-CD3 at N-terminus + tags) |
| 19 | GGGGSGGGGSGGGGS |
| | (Linker) |
| 20 | GSAPKPAPKPAPAPKPAPKPAP |
| | (linker) |
| 21 | GGGGSGGGGSGGGGSGGGGSAPKPAPKPAPAPKPAPKPAP |
| | (linker) |
| 22 | (GGGGS)n, wherein n = 1-6 |
| | (linker) |
| 23 | SSSSGSSSSGSAA |
| | (linker) |
| 24 | GGGGG |
| | (linker) |
| 25 | S(GGGGS)n, wherein n = 1-10 |
| | (linker) |
| 26 | (GGSG)n, wherein n = 1-5 |
| | (linker) |
| 27 | GSAT |
| | (linker) |
| 28 | (GGSGGS)n, wherein n = 1-5 |
| | (linker) |
| 29 | GGGGSGGGGSGGGGSGGGGS |
| | ((G₄S)₄ linker) |
| 30 | GSGSAGGSGSGGGS |
| | ((GS)₂AG₂SGSG₃S linker) |
| 31 | DYKDDDDK |
| | (FLAG Tag) |
| 32 | HHHHHH |
| | (His6 Tag) |
| 33 | GKPIPNPLLGLDST |
| | (V5 Tag) |
| 34 | WSHPQFEK |
| | (strep-tag) |
| 35 | EDQVDPRLIDGK |
| | (protein C tag) |
| 36 | EQKLISEEDL |
| | (Myc tag) |
| 37 | GLNDIFEAQKIEWHEGSGEQKLISEEDL |
| | (avitag-Myc (biotin-mediated)) |
| 38 | GLNDIFEAQKIEWHEGSGEQKLISEEDLHHHHHH |
| | (avitag-Myc-His (biotin-mediated)) |
| 39 | GSGSAGGGLNDIFEAQKIEWHEGSTGHHHHHHDYKDDDDK |
| | (Avitag sequence with His6 tag and Flag tag) |
| 40 | MKNHLLFWGVLAVFIKAVHVKAQEDERIVLVDNKCKCARITSRIIRSSEDPNEDIVERNIRIIVPLNN |
| | RENISDPTSPLRTRFVYHLSDLCKKCDPTEVELDNQIVTATQSNICDEDSATETCYTYDRNKCYTAVV |
| | PLVYGGETKMVETALTPDACYPD |
| | (human full-length J chain) |
| 41 | PTLYNVSLVMSDTAGTCY |
| | (human IgM tailpiece) |
| 42 | PTHVNVSVVMAEVDGTCY |
| | (human IgA tailpiece) |
| 43 | DXEXNPGP, where X = any amino acid |
| | (2A core sequence motif) |
| 44 | EGRGSLLTCGDVEENPGP |
| | (T2A sequence) |
| 45 | ATNFSLLKQAGDVEENPGP |
| | (P2A sequence) |
| 46 | QCTNYALLKLAGDVESNPGP |
| | (E2A sequence) |
| 47 | VKQTLNFDLLKLAGDVESNPGP |
| | (F2A sequence) |
| 48 | MEAPAQLLFLLLLWLPDTTG |
| | (Ig Kappa chain V-III region CLL signal peptide) |
| 49 | MNRGVPFRHLLLVLQLALLPAAT |
| | (signal peptide of human CD4 signal peptide) |
| 50 | METDTLLLWVLLLWVPGSTG |
| | (mouse Ig kappa chain V-III region signal peptide) |
| 51 | MVPCTLLLLLAAALAPTQTRA |
| | (mouse H-2Kb signal peptide) |
| 52 | MKWVTFISLLFLFSSAYS |
| | (human serum albumin signal peptide) |
| 53 | MYRMQLLSCIALSLALVTNS |
| | (human IL-2 signal peptide) |
| 54 | MAVMAPRTLLLLLSGALALTQTWA |
| | (human HLA-A*02:01 signal peptide) |
| 55 | MSRSVALAVLALLSLSGLEA |
| | (Human b2m signal peptide) |
| 56 | LPETGG |
| | (sortase recognition site) |
| 57 | KNEVEQSPQNLTAQEGEFITINCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKTVLDMRSMDFKSNSAVAWSNKSDFACANAFNNSIIPEDTFFPSPESS |
| | CDVKLVEKSIAELPPKVSVFVPPRDGFFGNPRKSKLICQATGFSPRQIQVSWLREGKQVGSGVTTDQV |
| | QAEAKESGPTTYKVTSTLTIKESDWLGQSMFTCRVDHRGLTFQQNASSMCVPDQDTAIRVFAIPPSFA |
| | SIFLTKSTKLTCLVTDLTTYDSVTISWTRQNGEAVKTHTNISESHPNATFSAVGEASICEDDWNSGER |
| | FTCTVTHTDLPSPLKQTISRPKGVALHRPDVYLLPPAREQLNLRESATITCLVTGFSPADVFVQWMQR |
| | GQPLSPEKYVTSAPMPEPQAPGRYFAHSILTVSEEEWNTGETYTCVVAHEALPNRVTERTVDKSTGKP |
| | TLYNVSLVMSDTAGTCY |
| 58 | (TCR D046C1a wt, connecting peptide linker, Cu2-4/amino acid sequence) |
| | aagaatgaagtggagcaatcacctcaaaacctaaccgctcaggagggtgaattcatcaccatcaactg |
| | tagctactccgtcggcatctccgcactgcattggctccagcagcatcctggaggtggcatcgtcagcc |
| | tcttcatgctgtcaagcggtaagaaaaagcatgggaggctgatcgccaccatcaatattcaggaaaaa |
| | cacagcagtttgcacattacagcttcccacccccgtgactctgccgtgtacatctgcgctgctgaccc |
| | ttctgacagttggggcaaattgcagttcggcgctggcacccaggtcgttgtgaccccggacattcaaa |
| | accccgatccggctgtctaccaactacgagactctaagagctctgacaaatccgtgtgcctttttacc |
| | gacttcgatagccaaacaaacgtgtctcagagcaaggacagcgacgtttatatcacagacaagactgt |
| | attggacatgcgctccatggacttcaagtccaatagcgctgttgcttggtccaataaaagtgatttcg |
| | cttgtgccaacgcttttaacaactctatcattcctgaggacacctttttcccttctcctgaaagttct |
| | tgtgatgttaagctggttgaaaagtccatcgcagaactgccacctaaagtcagcgtctttgtgcctcc |
| | ccgtgatggcttttttggcaacccccggaagagtaaactaatatgccaggccacaggatttagtccgc |
| | ggcagattcaggtgtcttggctccgggagggaaaacaggtcggaagtggagtgaccacagaccaagtg |
| | caggccgaggctaaggagagtggtcctacaacttacaaggttacaagtaccttgacaattaaagagtc |
| | cgattggctgggacagagtatgtttacatgtagagtggaccaccgtggtctcactttccagcagaatg |
| | ctagttcaatgtgcgttccggatcaggataccgcaattagagtgttcgccatccctccctcttttgct |
| | tccatcttcctgaccaagagcaccaagcttacatgcttggttaccgatcttacaacctatgattctgt |
| | cactatctcctggacacgccagaacggggaggccgtgaagacacatactaatatcagcgagagccatc |
| | ccaacgctacattctcagcagtgggcgaggcctctatctgcgaagacgactggaattcaggggaacgg |
| | ttcacgtgtactgtcacccatacagatctgccttcccctcttaagcagaccatttcacgaccgaaagg |
| | ggtggctcttcaccggcccgacgtgtacctactcccacctgcccgtgaacaactgaatctgcgcgaga |
| | gcgctaccattacctgcctggtaaccggatttagtcctgcagacgtctttgttcagtggatgcaacgc |
| | ggccagcccctctcccctgagaagtatgtgactagtgcccctatgcccgaaccccaggctcctggcag |
| | atattttgcccatagcatcctgacagtatcagaagaggagtggaacactggtgaaacctatacttgcg |
| | tggtggcacacgaggctttgccgaaccgcgtcacagaacgaaccgttgacaaaagcacaggtaagccc |
| | accctgtacaatgtcagcctcgtaatgtctgatacagcaggtacctgttat |
| | (TCR D046C1a wt, connecting peptide linker, Cu2-4/nucleotide sequence) |
| 59 | KNEVEQSPQNLTAQEGEFITINCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKTVLDMRSMDFKSNSAVAWSNKSDFACANAFNNSIIPEDTFFPSPESS |
| | CGGGGSGGGGSGGGGSIAELPPKVSVFVPPRDGFFGNPRKSKLICQATGFSPRQIQVSWLREGKQVGS |
| | GVTTDQVQAEAKESGPTTYKVTSTLTIKESDWLGQSMFTCRVDHRGLTFQQNASSMCVPDQDTAIRVF |
| | AIPPSFASIFLTKSTKLTCLVTDLTTYDSVTISWTRQNGEAVKTHTNISESHPNATFSAVGEASICED |
| | DWNSGERFTCTVTHTDLPSPLKQTISRPKGVALHRPDVYLLPPAREQLNLRESATITCLVTGFSPADV |
| | FVQWMQRGQPLSPEKYVTSAPMPEPQAPGRYFAHSILTVSEEEWNTGETYTCVVAHEALPNRVTERTV |
| | DKSTGKPTLYNVSLVMSDTAGTCY |
| | (TCR D046C1alpha wt, GS linker, Cu2-4/amino acid sequence) |
| 60 | aagaatgaagtggagcaatcacctcaaaacctaaccgctcaggagggtgaattcatcaccatcaactg |
| | tagctactccgtcggcatctccgcactgcattggctccagcagcatcctggaggtggcatcgtcagcc |
| | tcttcatgctgtcaagcggtaagaaaaagcatgggaggctgatcgccaccatcaatattcaggaaaaa |
| | cacagcagtttgcacattacagcttcccacccccgtgactctgccgtgtacatctgcgctgctgaccc |
| | ttctgacagttggggcaaattgcagttcggcgctggcacccaggtcgttgtgaccccggacattcaaa |
| | accccgatccggctgtctaccaactacgagactctaagagctctgacaaatccgtgtgcctttttacc |
| | gacttcgatagccaaacaaacgtgtctcagagcaaggacagcgacgtttatatcacagacaagactgt |
| | attggacatgcgctccatggacttcaagtccaatagcgctgttgcttggtccaataaaagtgatttcg |
| | cttgtgccaacgcttttaacaactctatcattcctgaggacacctttttcccttctcctgaaagttct |
| | tgtggcggaggtggttctgggggcgggggaagcggaggaggggggagtatcgcagaactgccacctaa |
| | agtcagcgtctttgtgcctccccgtgatggcttttttggcaacccccggaagagtaaactaatatgcc |
| | aggccacaggatttagtccgcggcagattcaggtgtcttggctccgggagggaaaacaggtcggaagt |
| | ggagtgaccacagaccaagtgcaggccgaggctaaggagagtggtcctacaacttacaaggttacaag |
| | taccttgacaattaaagagtccgattggctgggacagagtatgtttacatgtagagtggaccaccgtg |
| | gtctcactttccagcagaatgctagttcaatgtgcgttccggatcaggataccgcaattagagtgttc |
| | gccatccctccctcttttgcttccatcttcctgaccaagagcaccaagcttacatgcttggttaccga |
| | tcttacaacctatgattctgtcactatctcctggacacgccagaacggggaggccgtgaagacacata |
| | ctaatatcagcgagagccatcccaacgctacattctcagcagtgggcgaggcctctatctgcgaagac |
| | gactggaattcaggggaacggttcacgtgtactgtcacccatacagatctgccttcccctcttaagca |
| | gaccatttcacgaccgaaaggggtggctcttcaccggcccgacgtgtacctactcccacctgcccgtg |
| | aacaactgaatctgcgcgagagcgctaccattacctgcctggtaaccggatttagtcctgcagacgtc |
| | tttgttcagtggatgcaacgcggccagcccctctcccctgagaagtatgtgactagtgcccctatgcc |
| | cgaaccccaggctcctggcagatattttgcccatagcatcctgacagtatcagaagaggagtggaaca |
| | ctggtgaaacctatacttgcgtggtggcacacgaggctttgccgaaccgcgtcacagaacgaaccgtt |
| | gacaaaagcacaggtaagcccaccctgtacaatgtcagcctcgtaatgtctgatacagcaggtacctg |
| | ttat |
| 61 | (TCR D046C1alpha wt, GS linker, Cu2-4/nucleotide sequence) |
| | KNEVEQSPQNLTAQEGEFITINCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKTVLDMRSMDFKSNSAVAWSNKSDFACANAFNNSIIPEDTFFPSPESS |
| | CDVKLVEKSIRVFAIPPSFASIFLTKSTKLTCLVTDLTTYDSVTISWTRQNGEAVKTHTNISESHPNA |
| | TFSAVGEASICEDDWNSGERFTCTVTHTDLPSPLKQTISRPKGVALHRPDVYLLPPAREQLNLRESAT |
| | ITCLVTGFSPADVFVQWMQRGQPLSPEKYVTSAPMPEPQAPGRYFAHSILTVSEEEWNTGETYTCVVA |
| | HEALPNRVTERTVDKSTGKPTLYNVSLVMSDTAGTCY |
| 62 | (TCR D046C1a wt, connecting peptide linker, Cu3-4/amino acid sequence) |
| | aagaatgaagtggagcaatcacctcaaaacctaaccgctcaggagggtgaattcatcaccatcaactg |
| | tagctactccgtcggcatctccgcactgcattggctccagcagcatcctggaggtggcatcgtcagcc |
| | tcttcatgctgtcaagcggtaagaaaaagcatgggaggctgatcgccaccatcaatattcaggaaaaa |
| | cacagcagtttgcacattacagcttcccacccccgtgactctgccgtgtacatctgcgctgctgaccc |
| | ttctgacagttggggcaaattgcagttcggcgctggcacccaggtcgttgtgaccccggacattcaaa |
| | accccgatccggctgtctaccaactacgagactctaagagctctgacaaatccgtgtgcctttttacc |
| | gacttcgatagccaaacaaacgtgtctcagagcaaggacagcgacgtttatatcacagacaagactgt |
| | attggacatgcgctccatggacttcaagtccaatagcgctgttgcttggtccaataaaagtgatttcg |
| | cttgtgccaacgcttttaacaactctatcattcctgaggacacctttttcccttctcctgaaagttct |
| | tgtgatgttaagctggttgaaaagtccatccgagtattcgctatcccacccagtttcgcctctatctt |
| | ccttaccaaatctactaaactgacatgcttggtcactgatctaaccacttacgattctgtgacaattt |
| | cctggacaaggcagaacggagaggcagtaaagactcatacgaatatttcagaatcccatcccaacgcc |
| | acgttttccgccgttggcgaggcctccatttgtgaggacgattggaatagcggagagcgctttacctg |
| | cacagtgacgcatacagatctgcctagcccactcaagcagactatctctcgcccaaagggggtggctc |
| | tgcataggcctgacgtatatctccttccacccgctcgagagcagctgaacttgagagaatccgcaact |
| | attacttgcttggtgacgggcttttctcccgccgatgtcttcgtgcagtggatgcagcgtggacagcc |
| | tctctctcctgagaaatacgtaacatctgcccccatgcccgagcctcaggctcctggtcgttattttg |
| | ctcacagtatcctgaccgtcagcgaagaggaatggaacactggagagacctacacctgtgtcgtggct |
| | catgaagctctgcctaacagggtcaccgagcgcaccgtggacaaaagcactggcaaaccgaccctgta |
| | taacgtgtcactcgtgatgagcgacacagccggcacttgctat |
| | (TCR D046C1a wt, connecting peptide linker, Cu3-4/nucleotide sequence) |
| 63 | KNEVEQSPQNLTAQEGEFITINCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKTVLDMRSMDFKSNSAVAWSNKSDFACANAFNNSIIPEDTFFPSPESS |
| | CGGGGSGGGGSGGGGSIRVFAIPPSFASIFLTKSTKLTCLVTDLTTYDSVTISWTRQNGEAVKTHTNI |
| | SESHPNATFSAVGEASICEDDWNSGERFTCTVTHTDLPSPLKQTISRPKGVALHRPDVYLLPPAREQL |
| | NLRESATITCLVTGFSPADVFVQWMQRGQPLSPEKYVTSAPMPEPQAPGRYFAHSILTVSEEEWNTGE |
| | TYTCVVAHEALPNRVTERTVDKSTGKPTLYNVSLVMSDTAGTCY |
| | (TCR D046C1a wt, G-S linker, Cu3-4/amino acid sequence) |
| 64 | aagaatgaagtggagcaatcacctcaaaacctaaccgctcaggagggtgaattcatcaccatcaactg |
| | tagctactccgtcggcatctccgcactgcattggctccagcagcatcctggaggtggcatcgtcagcc |
| | tcttcatgctgtcaagcggtaagaaaaagcatgggaggctgatcgccaccatcaatattcaggaaaaa |
| | cacagcagtttgcacattacagcttcccacccccgtgactctgccgtgtacatctgcgctgctgaccc |
| | ttctgacagttggggcaaattgcagttcggcgctggcacccaggtcgttgtgaccccggacattcaaa |
| | accccgatccggctgtctaccaactacgagactctaagagctctgacaaatccgtgtgcctttttacc |
| | gacttcgatagccaaacaaacgtgtctcagagcaaggacagcgacgtttatatcacagacaagactgt |
| | attggacatgcgctccatggacttcaagtccaatagcgctgttgcttggtccaataaaagtgatttcg |
| | cttgtgccaacgcttttaacaactctatcattcctgaggacacctttttcccttctcctgaaagttct |
| | tgtggcggaggtggttctgggggcgggggaagcggaggaggggggagtatccgagtattcgctatccc |
| | acccagtttcgcctctatcttccttaccaaatctactaaactgacatgcttggtcactgatctaacca |
| | cttacgattctgtgacaatttcctggacaaggcagaacggagaggcagtaaagactcatacgaatatt |
| | tcagaatcccatcccaacgccacgttttccgccgttggcgaggcctccatttgtgaggacgattggaa |
| | tagcggagagcgctttacctgcacagtgacgcatacagatctgcctagcccactcaagcagactatct |
| | ctcgcccaaagggggtggctctgcataggcctgacgtatatctccttccacccgctcgagagcagctg |
| | aacttgagagaatccgcaactattacttgcttggtgacgggcttttctcccgccgatgtcttcgtgca |
| | gtggatgcagcgtggacagcctctctctcctgagaaatacgtaacatctgcccccatgcccgagcctc |
| | aggctcctggtcgttattttgctcacagtatcctgaccgtcagcgaagaggaatggaacactggagag |
| | acctacacctgtgtcgtggctcatgaagctctgcctaacagggtcaccgagcgcaccgtggacaaaag |
| | cactggcaaaccgaccctgtataacgtgtcactcgtgatgagcgacacagccggcacttgctat |
| | (TCR D046C1a wt, G-S linker, Cu3-4/nucleotide sequence) |
| 65 | FTVTVPKDLYVVEYGSNMTIECKFPVEKQLDLAALIVYWEMEDKNIIQFVHGEEDLKVQHSSYRQRAR |
| | LLKDQLSLGNAALQITDVKLQDAGVYRCMISYGGADYKRITVKVNAPYNKINQRILVVDPVTSEHELT |
| | CQAEGYPKAEVIWTSSDHQVLSGKTTTTNSKREEKLFNVTSTLRINTTTNEIFYCTFRRLDPEENHTA |
| | ELVIPELPLAHPPNER |
| | (PDL1 ECD) |
| 66 | FTVTVPKDLYVVEYGSNMTIECKFPVEKQLDLAALIVYWEMEDKNIIQFVHGEEDLKVQHSSYRQRAR |
| | LLKDQLSLGNAALQITDVKLQDAGVYRCMISYGGADYKRITVKVNA |
| | (PDL1 D1) |
| 67 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSFTVTVPKDLYVVEYGSNMTIECKFPVEKQLDLAALIVYWEMEDKNIIQFVHG |
| | EEDLKVQHSSYRQRARLLKDQLSLGNAALQITDVKLQDAGVYRCMISYGGADYKRITVKVNAPYNKIN |
| | QRILVVDPVTSEHELTCQAEGYPKAEVIWTSSDHQVLSGKTTTTNSKREEKLFNVTSTLRINTTTNEI |
| | FYCTFRRLDPEENHTAELVIPELPLAHPPNER |
| | (Jch-(G4S)₃-PDL1ECD amino acid sequence) |
| 68 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSFTVTVPKDLYVVEYGSNMTIECKFPVEKQLDLAALIVYWEMEDKNIIQFVHG |
| | EEDLKVQHSSYRQRARLLKDQLSLGNAALQITDVKLQDAGVYRCMISYGGADYKRITVKVNA |
| | (Jch-(G4S)₃-PDL1D1 amino acid sequence) |
| 69 | KNEVEQSPQNLTAQEGEFITIQCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKCVLDMRSMDFKSNSAVAWSQKSDFACANAFQNSIIPEDTFFPSPESS |
| | CIAELPPKVSVFVPPRDGFFGNPRKSKLICQATGFSPRQIQVSWLREGKQVGSGVTTDQVQAEAKESG |
| | PTTYKVTSTLTIKESDWLGQSMFTCRVDHRGLTFQQNASSMCVPDQDTAIRVFAIPPSFASIFLTKST |
| | KLTCLVTDLTTYDSVTISWTRQNGEAVKTHTNISESHPNATFSAVGEASICEDDWNSGERFTCTVTHT |
| | DLPSPLKQTISRPKGVALHRPDVYLLPPAREQLNLRESATITCLVTGFSPADVFVQWMQRGQPLSPEK |
| | YVTSAPMPEPQAPGRYFAHSILTVSEEEWNTGETYTCVVAHEALPNRVTERTVDKSTGKPTLYNVSLV |
| | MSDTAGTCY |
| | (TCR-IgM (removal of three N-linked glycosylation sites by mutation |
| | of NàQ, introduction of cysteine to form DS with alpha . . . |
| | corresponds to DNA1944/PS832 D046Va-Ca (mt), L2 = IAELP, Cu2-4)) |
| 70 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSDYKDDDDKHHHHHHHH |
| | (Jch-Flag-His8) |
| 71 | KNEVEQSPQNLTAQEGEFITIQCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKCVLDMRSMDFKSNSAVAWSQKSDFACANAFQNSIIPEDTFFPSPESS |
| | CDVKLVEKSDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTVDKS |
| | RWQQGNVFSCSVMHEALHNHYTQKSLSLSPGK |
| | (TCR D046C1 alpha chain-IgG) |
| 72 | ELAGIGILTV |
| | (ELA peptide) |
| 73 | QVQLQQSGAELARPGASVKMSCKASGYTFTRYTMHWVKQRPGQGLEWIGYINPSRGYTNYNQKFKDKA |
| | TLTTDKSSSTAYMQLSSLTSEDSAVYYCARYYDDHYSLDYWGQGTTLTVSSGGGGSGGGGSGGGGSQI |
| | VLTQSPAIMSASPGEKVTMTCSASSSVSYMNWYQQKSGTSPKRWIYDTSKLASGVPAHFRGSGSGTSY |
| | SLTISGMEAEDAATYYCQQWSSNPFTFGSGTKLEIKGGGGSGGGGSGGGGSQEDERIVLVDNKCKCAR |
| | ITSRIIRSSEDPNEDIVERNIRIIVPLNNRENISDPTSPLRTRFVYHLSDLCKKCDPTEVELDNQIVT |
| | ATQSNICDEDSATETCYTYDRNKCYTAVVPLVYGGETKMVETALTPDACYPDGGGGSGGGGSDYKDDD |
| | DKHHHHHHHH |
| | (Anti-CD3scFv-Linker-Jch-Flag-His) |
| 74 | FTVTVPKDLYVVEYGSNMTIECKFPVEKQLDLAALIVYWEMEDKNIIQFVHGEEDLKVQHSSYRQRAR |
| | LLKDQLSLGNAALQITDVKLQDAGVYRCMISYGGADYKRITVKVNAPYNKINQRILVVDPVTSEHELT |
| | CQAEGYPKAEVIWTSSDHQVLSGKTTTTNSKREEKLFNVTSTLRINTTTNEIFYCTFRRLDPEENHTA |
| | ELVIPELPLAHPPNERGGGGSGGGGSGGGGSGGGGSGGGGSQEDERIVLVDNKCKCARITSRIIRSSE |
| | DPNEDIVERNIRIIVPLNNRENISDPTSPLRTRFVYHLSDLCKKCDPTEVELDNQIVTATQSNICDED |
| | SATETCYTYDRNKCYTAVVPLVYGGETKMVETALTPDACYPDGGGGSGGGGSDYKDDDDKHHHHHHHH |
| | (PD-L1-ECD-Jch-Flag-His) |
| 75 | HHHHHHHH |
| | (His8 tag) |
| 76 | MIQRTPKIQVYSRHPAENGKSNFLNCYVSGFHPSDIEVDLLKNGERIEKVEHSDLSFSKDWSFYLLYY |
| | TEFTPTEKDEYACRVNHVTLSQPKIVKWDRDM |
| | (B2m seq) |
| 77 | GSHSMRYFFTSVSRPGRGEPRFIAVGYVDDTQFVRFDSDAASQRMEPRAPWIEQEGPEYWDGETRKVK |
| | AHSQTHRVDLGTLRGYYNQSEAGSHTVQRMYGCDVGSDWRFLRGYHQYAYDGKDYIALKEDLRSWTAA |
| | DMAAQTTKHKWEAAHVAEQLRAYLEGTCVEWLRRYLENGKETLQRTDAPKTHMTHHAVSDHEATLRCW |
| | ALSFYPAEITLTWQRDGEDQTQDTELVETRPAGDGTFQKWAAVVVPSGQEQRYTCHVQHEGLPKPLTL |
| | RWEPSSGSGSAGGGLNDIFEAQKIEWHEGSTGHHHHHHDYKDDDDK |
| | (HLA-A*02 soluble-Avi tag-His6-Flag) |
| 78 | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | DPSPLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (IgM Cm2-Cm3-Cm4-TP L310D) |
| 79 | IRVFAIPPSFASIFLTKSTKLTCLVTDLTTYDSVTISWTRQNGEAVKTHTNISESHPNATFSAVGEAS |
| | ICEDDWNSGERFTCTVTHTDDPSPLKQTISRPKGVALHRPDVYLLPPAREQLNLRESATITCLVTGFS |
| | PADVFVQWMQRGQPLSPEKYVTSAPMPEPQAPGRYFAHSILTVSEEEWNTGETYTCVVAHEALPNRVT |
| | ERTVDKSTGKPTLYNVSLVMSDTAGTCY |
| | (IgM Cm3-Cm4-TP L310D) |
| 80 | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | LASSLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (IgM Cm2-Cm3-Cm4-TP P311A, P313S) |
| 81 | IRVFAIPPSFASIFLTKSTKLTCLVTDLTTYDSVTISWTRQNGEAVKTHTNISESHPNATFSAVGEAS |
| | ICEDDWNSGERFTCTVTHTDLASSLKQTISRPKGVALHRPDVYLLPPAREQLNLRESATITCLVTGFS |
| | PADVFVQWMQRGQPLSPEKYVTSAPMPEPQAPGRYFAHSILTVSEEEWNTGETYTCVVAHEALPNRVT |
| | ERTVDKSTGKPTLYNVSLVMSDTAGTCY |
| | (IgM Cm3-Cm4-TP P311A, P313S) |
| 82 | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | LGSPLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (IgM Cm2-Cm3-Cm4-TP P311G) |
| 83 | IRVFAIPPSFASIFLTKSTKLTCLVTDLTTYDSVTISWTRQNGEAVKTHTNISESHPNATFSAVGEAS |
| | ICEDDWNSGERFTCTVTHTDLGSPLKQTISRPKGVALHRPDVYLLPPAREQLNLRESATITCLVTGFS |
| | PADVFVQWMQRGQPLSPEKYVTSAPMPEPQAPGRYFAHSILTVSEEEWNTGETYTCVVAHEALPNRVT |
| | ERTVDKSTGKPTLYNVSLVMSDTAGTCY |
| | (IgM Cm3-Cm4-TP P311G) |
| 84 | KNEVEQSPQNLTAQEGEFITIQCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKCVLDMRSMDFKSNSAVAWSQKSDFACANAFQNSIIPEDTFFPSPESS |
| | CIAELPPKVSVFVPPRDGFFGNPRKSKLICQATGFSPRQIQVSWLREGKQVGSGVTTDQVQAEAKESG |
| | PTTYKVTSTLTIKESDWLGQSMFTCRVDHRGLTFQQNASSMCVPDQDTAIRVFAIPPSFASIFLTKST |
| | KLTCLVTDLTTYDSVTISWTRQNGEAVKTHTNISESHPNATFSAVGEASICEDDWNSGERFTCTVTHT |
| | DLASSLKQTISRPKGVALHRPDVYLLPPAREQLNLRESATITCLVTGFSPADVFVQWMQRGQPLSPEK |
| | YVTSAPMPEPQAPGRYFAHSILTVSEEEWNTGETYTCVVAHEALPNRVTERTVDKSTGKPTLYNVSLV |
| | MSDTAGTCY |
| | (TCR D046C1 α chain-IgM2-4 comp mt (TRAV: N22Q; TRAC: T84C, N90Q, N109Q) |
| | (Ig MP311A, P313S)) |
| 85 | KNEVEQSPQNLTAQEGEFITIQCSYSVGISALHWLQQHPGGGIVSLFMLSSGKKKHGRLIATINIQEK |
| | HSSLHITASHPRDSAVYICAADPSDSWGKLQFGAGTQVVVTPDIQNPDPAVYQLRDSKSSDKSVCLFT |
| | DFDSQTNVSQSKDSDVYITDKCVLDMRSMDFKSNSAVAWSQKSDFACANAFQNSIIPEDTFFPSPESS |
| | CIRVFAIPPSFASIFLTKSTKLTCLVTDLTTYDSVTISWTRQNGEAVKTHTNISESHPNATFSAVGEA |
| | SICEDDWNSGERFTCTVTHTDLASSLKQTISRPKGVALHRPDVYLLPPAREQLNLRESATITCLVTGF |
| | SPADVFVQWMQRGQPLSPEKYVTSAPMPEPQAPGRYFAHSILTVSEEEWNTGETYTCVVAHEALPNRV |
| | TERTVDKSTGKPTLYNVSLVMSDTAGTCY |
| | (TCR D046C1 α chain-IgM3-4 comp mt (TRAV: N22Q; TRAC: T84C, N90Q, N109Q) |
| | (IgM P311A, P313S)) |
| 86 | DGGITQSPKYLFRKEGQNVTLSCEQNLNHDAMYWYRQDPGQGLRLIYYSWAQGDFQKGDIAEGYSVSR |
| | EKKESFPLTVTSAQKNPTAFYLCASSWGAPYEQYFGPGTRLTVTEDLKNVFPPEVAVFEPSKAEISRT |
| | QKATLVCLATGFYPPHVELSWWVNGKEVHDGVCTDPQPLKEQPALQDSRYALSSRLRVSATFWQDPRN |
| | HFRCQVQFYGLSENDEWTQDRAKPVTQIVSAEAWGRADC |
| | (AM-gp-beta (affinity matured gp100 TCR beta with deglycosylation and |
| | stabilizing mutations) (TRBC2: E11K, H15.1R, D35P, S45.5D, N85.6Q, |
| | S79C, C85.1A) (PS1421)) |
| 87 | AQQGEEDPQALSIQEGENATMQCSYKTSINNLQWYRQNSGRGLVHLILIRSNEREKHSGRLRVTLDTS |
| | KKSSSLLITASRAADTASYFCATDGSTPMQFGKGTRLSVIANIQKPDPAVYQLRDSKSSDKFVCLFTD |
| | FDSQINVSQSKDSDVYITDKCVLDMRSMDFKSNSAVAWSQKSDFTCANAFQNSIIPEDTFFPSPESSC |
| | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | LPSPLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (AM-gp-alpha-IgMCu2-4 (affinity matured gp100 TCR alpha with |
| | deglycosylation and stabilizing mutations fused to IgM Cu2-4) (TRAC: |
| | S21F, T37I, A103T, N22Q, TRAC: N90Q, N109Q, T84C) (PS1416)) |
| 88 | SQQGEEDPQALSIQEGENATMNCSYKTSINNLQWYRQNSGRGLVHLILIRSNEREKHSGRLRVTLDTS |
| | KKSSSLLITASRAADTASYFCATDGDTPLVFGKGTRLSVIANIQKPDPAVYQLRDSKSSDKFVCLFTD |
| | FDSQINVSQSKDSDVYITDKCVLDMRSMDFKSNSAVAWSNKSDFTCANAFNNSIIPEDTFFPSPESSC |
| | IAELPPKVSVFVPPRDGFFGNPRKSKLICQATGFSPRQIQVSWLREGKQVGSGVTTDQVQAEAKESGP |
| | TTYKVTSTLTIKESDWLGQSMFTCRVDHRGLTFQQNASSMCVPDQDTAIRVFAIPPSFASIFLTKSTK |
| | LTCLVTDLTTYDSVTISWTRQNGEAVKTHTNISESHPNATFSAVGEASICEDDWNSGERFTCTVTHTD |
| | LPSPLKQTISRPKGVALHRPDVYLLPPAREQLNLRESATITCLVTGFSPADVFVQWMQRGQPLSPEKY |
| | VTSAPMPEPQAPGRYFAHSILTVSEEEWNTGETYTCVVAHEALPNRVTERTVDKSTGKPTLYNVSLVM |
| | SDTAGTCY |
| | (Par-gp-alpha-IgMCu2-4 (Parent gp100 TCR alpha with stabilizing |
| | mutations fused to IgM Cu2-4) (TRAC: S21F, T37I, A103T, T84C) (PS1465)) |
| 89 | DGGITQSPKYLFRKEGQNVTLSCEQNLNHDAMYWYRQDPGQGLRLIYYSQIVNDFQKGDIAEGYSVSR |
| | EKKESFPLTVTSAQKNPTAFYLCASSIGGPYEQYFGPGTRLTVTEDLKNVFPPEVAVFEPSKAEISRT |
| | QKATLVCLATGFYPPHVELSWWVNGKEVHDGVCTDPQPLKEQPALNDSRYALSSRLRVSATFWQDPRN |
| | HFRCQVQFYGLSENDEWTQDRAKPVTQIVSAEAWGRADC |
| | (Par-gp-beta (Parent gp100 TCR beta with stabilizing mutations) |
| | (TRBC2: E11K, H15.1R, D35P, S45.5D, S79C, C85.1A) (PS1503)) |
| 90 | SQQGEEDPQALSIQEGENATMNCSYKTSINNLQWYRQNSGRGLVHLILIRSNEREKHSGRLRVTLDTS |
| | KKSSSLLITASRAADTASYFCATDGDTPLVFGKGTRLSVIANIQKPDPAVYQLRDSKSSDKFVCLFTD |
| | FDSQINVSQSKDSDVYITDKCVLDMRSMDFKSNSAVAWSNKSDFTCANAFNNSIIPEDTFFPSPESSC |
| | DKTHTCPPCPAPDQDTAIRVFAIPPSFASIFLTKSTKLTCLVTDLTTYDSVTISWTRQNGEAVKTHTN |
| | ISESHPNATFSAVGEASICEDDWNSGERFTCTVTHTDLPSPLKQTISRPKGVALHRPDVYLLPPAREQ |
| | LNLRESATITCLVTGFSPADVFVQWMQRGQPLSPEKYVTSAPMPEPQAPGRYFAHSILTVSEEEWNTG |
| | ETYTCVVAHEALPNRVTERTVDKSTGKPTLYNVSLVMSDTAGTCY |
| | (Par-gp-alpha-g1Hn-IgMCu3-4 (Parent gp100 TCR alpha with stabilizing |
| | mutations fused to IgG1Hinge-IgMCu3-4) (TRAC: S21F, T37I, A103T, T84C) |
| | (PS1638)) |
| 91 | AQQGEEDPQALSIQEGENATMQCSYKTSINNLQWYRQNSGRGLVHLILIRSNEREKHSGRLRVTLDTS |
| | KKSSSLLITASRAADTASYFCATDGSTPMQFGKGTRLSVIANIQKPDPAVYQLRDSKSSDKFVCLFTD |
| | FDSQINVSQSKDSDVYITDKCVLDMRSMDFKSNSAVAWSQKSDFTCANAFQNSIIPEDTFFPSPESSC |
| | DKTHTCPPCPAPDQDTAIRVFAIPPSFASIFLTKSTKLTCLVTDLTTYDSVTISWTRQNGEAVKTHTN |
| | ISESHPNATFSAVGEASICEDDWNSGERFTCTVTHTDLPSPLKQTISRPKGVALHRPDVYLLPPAREQ |
| | LNLRESATITCLVTGFSPADVFVQWMQRGQPLSPEKYVTSAPMPEPQAPGRYFAHSILTVSEEEWNTG |
| | ETYTCVVAHEALPNRVTERTVDKSTGKPTLYNVSLVMSDTAGTCY |
| | (AM-gp-alpha-g1Hn-IgMCu3-4 (affinity matured gp100 TCR alpha with |
| | deglycosylation and stabilizing mutations fused to IgG1Hinge-IgMCu3- |
| | 4) (TRAC: S21F, T37I, A103T) (TRAV: N22Q, TRAC: N90Q, N109Q) (Disulfile |
| | stabilized TRAC: T84C) (PS1632)) |
| 92 | AIQMTQSPSSLSASVGDRVTITCRASQDIRNYLNWYQQKPGKAPKLLIYYTSRLESGVPSRFSGSGSG |
| | TDYTLTISSLQPEDFATYYCQQGNTLPWTFGQGTKVEIKGGGGSGGGGSGGGGSGGGGSGGGSEVQLV |
| | ESGGGLVQPGGSLRLSCAASGYSFTGYTMNWVRQAPGKGLEWVALINPYKGVSTYNQKFKDRFTISVD |
| | KSKNTAYLQMNSLRAEDTAVYYCARSGYYGDSDWYFDVWGQGTLVTVSSGGGGSDGGITQSPKYLFRK |
| | EGQNVTLSCEQNLNHDAMYWYRQDPGQGLRLIYYSWAQGDFQKGDIAEGYSVSREKKESFPLTVTSAQ |
| | KNPTAFYLCASSWGAPYEQYFGPGTRLTVTEDLKNVFPPEVAVFEPSKAEISRTQKATLVCLATGFYP |
| | PHVELSWWVNGKEVHDGVCTDPQPLKEQPALQDSRYALSSRLRVSATFWQDPRNHFRCQVQFYGLSEN |
| | DEWTQDRAKPVTQIVSAEAWGRADCGSDYKDDDDKLEHHHHHHHH |
| | (U1v9-AM-gp-beta (scFv-UCHT1v9 (DtoA)-)-affinity matured-TCR-beta with |
| | deglycosylation and stabilizing mutations + tags) (TRBC2: E11K, |
| | H15.1R, D35P, S45.5D), (TRBC2: N85.6Q), (TRBC2: S79C, C85.1A) |
| | (PL373_PS1364)) |
| 93 | AQQGEEDPQALSI QEGENATMQCSYKTSINNLQWYRQNSGRGLVHLILIRSNEREKHSGRLRVTLDTS |
| | KKSSSLLITASRAADTASYFCATDGSTPMQFGKGTRLSVIANIQKPDPAVYQLRDSKSSDKFVCLFTD |
| | FDSQINVSQSKDSDVYITDKCVLDMRSMDFKSNSAVAWSQKSDFTCANAFQNSIIPEDTFFPSPESSC |
| | (AM-gp-alpha (affinity matured gp100 TCR alpha with deglycosylation |
| | and stabilizing mutations) (TRAC: S21F, T37I, A103T), (TRAV: N22Q, |
| | TRAC: N90Q, N109Q), (Disulfide stabilized TRAC: T84C) (PL373_PS1365)) |
| 94 | SQQGEEDPQALSIQEGENATMNCSYKTSINNLQWYRQNSGRGLVHLILIRSNEREKHSGRLRVTLDTS |
| | KKSSSLLITASRAADTASYFCATDGDTPLVFGKGTRLSVIANIQKPDPAVYQLRDSKSSDKFVCLFTD |
| | FDSQINVSQSKDSDVYITDKCVLDMRSMDFKSNSAVAWSNKSDFTCANAFNNSIIPEDTFFPSPESSC |
| | GGGGSGGGGSGGGGSAIQMTQSPSSLSASVGDRVTITCRASQDIRNYLNWYQQKPGKAPKLLIYYTSR |
| | LESGVPSRFSGSGSGTDYTLTISSLQPEDFATYYCQQGNTLPWTFGQGTKVEIKGGGGSGGGGSGGGG |
| | SGGGGSGGGSEVQLVESGGGLVQPGGSLRLSCAASGYSFTGYTMNWVRQAPGKGLEWVALINPYKGVS |
| | TYNQKFKDRFTISVDKSKNTAYLQMNSLRAEDTAVYYCARSGYYGDSDWYFDVWGQGTLVTVSSGSDY |
| | KDDDDKLEHHHHHHHH |
| | (Par-gp-alpha-U1v9 (Parent gp100 TCR alpha with stabilizing mutations |
| | fused to scFv U1v9 (DtoA) + tags) (TRAC: S21F, T37I, A103T), (Disulfide |
| | stabilized with TRAC: T84C) (PL427 PS1463)) |
| 95 | DKTHTCPPCPAPDQDTAIRVFAIPPSFASIFLTKSTKLTCLVTDLTTYDSVTISWTRQNGEAVKTHTN |
| | ISESHPNATFSAVGEASICEDDWNSGERFTCTVTHTDLPSPLKQTISRPKGVALHRPDVYLLPPAREQ |
| | LNLRESATITCLVTGFSPADVFVQWMQRGQPLSPEKYVTSAPMPEPQAPGRYFAHSILTVSEEEWNTG |
| | ETYTCVVAHEALPNRVTERTVDKSTGKPTLYNVSLVMSDTAGTCY |
| | (hingeIgG-IgMCu3-4) |
| 96 | AIQMTQSPSSLSASVGDRVTITCRASQDIRNYLNWYQQKPGKAPKLLIYYTSRLESGVPSRFSGSGSG |
| | TDYTLTISSLQPEDFATYYCQQGNTLPWTFGQGTKVEIKGGGGSGGGGSGGGGSGGGGSGGGSEVQLV |
| | ESGGGLVQPGGSLRLSCAASGYSFTGYTMNWVRQAPGKGLEWVALINPYKGVSTYNQKFKDRFTISVD |
| | KSKNTAYLQMNSLRAEDTAVYYCARSGYYGDSDWYFDVWGQGTLVTVSSGGGGSGGGGSGGGGSGGGG |
| | SGGGGSQEDERIVLVDNKCKCARITSRIIRSSEDPNEDIVERNIRIIVPLNNRENISDPTSPLRTRFV |
| | YHLSDLCKKCDPTEVELDNQIVTATQSNICDEDSATETCYTYDRNKCYTAVVPLVYGGETKMVETALT |
| | PDACYPDGGGGSGGGGSDYKDDDDKHHHHHHHH |
| | (Human Ig Jch with anti-CD3 (UCHT1v9 (DIA)) at N-terminus + tags |
| | (PS1429)) |
| 97 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSGGGGSGGGGSAIQMTQSPSSLSASVGDRVTITCRASQDIRNYLNWYQQKPGK |
| | APKLLIYYTSRLESGVPSRFSGSGSGTDYTLTISSLQPEDFATYYCQQGNTLPWTFGQGTKVEIKGGG |
| | GSGGGGSGGGGSGGGGSGGGSEVQLVESGGGLVQPGGSLRLSCAASGYSFTGYTMNWVRQAPGKGLEW |
| | VALINPYKGVSTYNQKFKDRFTISVDKSKNTAYLQMNSLRAEDTAVYYCARSGYYGDSDWYFDVWGQG |
| | TLVTVSSGGGGSGGGGSDYKDDDDKHHHHHHHH |
| | (Human Ig Jch with anti-CD3 (UCHT1v9 (DIA)) at C-terminus + tags) |
| 98 | YLEPGPVTA |
| | (gp100pep Peptide) |
| 99 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSQVQLQQSGAELARPGASVKMSCKASGYTFTRYTMHWVKQRPGQGLEWIGYIN |
| | PSRGYTNYNQKFKDKATLTTDKSSSTAYMQLSSLTSEDSAVYYCARYYDDHYSLDYWGQGTTLTVSSG |
| | GGGSGGGGSGGGGSQIVLTQSPAIMSASPGEKVTMTCSASSSVSYMNWYQQKSGTSPKRWIYDTSKLA |
| | SGVPAHFRGSGSGTSYSLTISGMEAEDAATYYCQQWSSNPFTFGSGTKLEIK |
| | (Jch-OKT3) |
| 100 | QVQLQQSGAELARPGASVKMSCKASGYTFTRYTMHWVKQRPGQGLEWIGYINPSRGYTNYNQKFKDKA |
| | TLTTDKSSSTAYMQLSSLTSEDSAVYYCARYYDDHYSLDYWGQGTTLTVSSGGGGSGGGGSGGGGSQI |
| | VLTQSPAIMSASPGEKVTMTCSASSSVSYMNWYQQKSGTSPKRWIYDTSKLASGVPAHFRGSGSGTSY |
| | SLTISGMEAEDAATYYCQQWSSNPFTFGSGTKLEIKGGGGSGGGGSGGGGSQEDERIVLVDNKCKCAR |
| | ITSRIIRSSEDPNEDIVERNIRIIVPLNNRENISDPTSPLRTRFVYHLSDLCKKCDPTEVELDNQIVT |
| | ATQSNICDEDSATETCYTYDRNKCYTAVVPLVYGGETKMVETALTPDACYPD |
| | (OKT3-Jch) |
| 101 | AIQMTQSPSSLSASVGDRVTITCRASQDIRNYLNWYQQKPGKAPKLLIYYTSRLESGVPSRFSGSGSG |
| | TDYTLTISSLQPEDFATYYCQQGNTLPWTFGQGTKVEIKGGGGSGGGGSGGGGSGGGGSGGGSEVQLV |
| | ESGGGLVQPGGSLRLSCAASGYSFTGYTMNWVRQAPGKGLEWVALINPYKGVSTYNQKFKDRFTISVD |
| | KSKNTAYLQMNSLRAEDTAVYYCARSGYYGDSDWYFDVWGQGTLVTVSS |
| | (U1v9(D1A) scFv) |
| 102 | AIQMTQSPSSLSASVGDRVTITCRASQDIRNYLNWYQQKPGKAPKLLIYYTSRLESGVPSRFSGSGSG |
| | TDYTLTISSLQPEDFATYYCQQGNTLPWTFGQGTKVEIKGGGGSGGGGSGGGGSGGGGSGGGSEVQLV |
| | ESGGGLVQPGGSLRLSCAASGYSFTGYTMNWVRQAPGKGLEWVALINPYKGVSTYNQKFKDRFTISVD |
| | KSKNTAYLQMNSLRAEDTAVYYCARSGYYGDSDWYFDVWGQGTLVTVSSGGGGSDGGITQSPKYLFRK |
| | EGQNVTLSCEQNLNHDAMYWYRQDPGQGLRLIYYSWAQGDFQKGDIAEGYSVSREKKESFPLTVTSAQ |
| | KNPTAFYLCASSWGAPYEQYFGPGTRLTVTEDLKNVFPPEVAVFEPSKAEISRTQKATLVCLATGFYP |
| | PHVELSWWVNGKEVHDGVCTDPQPLKEQPALQDSRYALSSRLRVSATFWQDPRNHFRCQVQFYGLSEN |
| | DEWTQDRAKPVTQIVSAEAWGRADC |
| | (U1v9-AM-gp-beta (scFv-UCHT1v9 (DtoA)-affinity matured-TCR-beta with |
| | deglycosylation and stabilizing mutations) (TRBC2: E11K, H15.1R, D35P, |
| | S45.5D), (TRBC2: N85.6Q), (TRBC2: S79C, C85.1A) (PL373_PS1364)) |
| 103 | SQQGEEDPQALSIQEGENATMNCSYKTSINNLQWYRQNSGRGLVHLILIRSNEREKHSGRLRVTLDTS |
| | KKSSSLLITASRAADTASYFCATDGDTPLVFGKGTRLSVIANIQKPDPAVYQLRDSKSSDKFVCLFTD |
| | FDSQINVSQSKDSDVYITDKCVLDMRSMDFKSNSAVAWSNKSDFTCANAFNNSIIPEDTFFPSPESSC |
| | GGGGSGGGGSGGGGSAIQMTQSPSSLSASVGDRVTITCRASQDIRNYLNWYQQKPGKAPKLLIYYTSR |
| | LESGVPSRFSGSGSGTDYTLTISSLQPEDFATYYCQQGNTLPWTFGQGTKVEIKGGGGSGGGGSGGGG |
| | SGGGGSGGGSEVQLVESGGGLVQPGGSLRLSCAASGYSFTGYTMNWVRQAPGKGLEWVALINPYKGVS |
| | TYNQKFKDRFTISVDKSKNTAYLQMNSLRAEDTAVYYCARSGYYGDSDWYFDVWGQGTLVTVSS |
| | (Par-gp-alpha-U1v9 (Parent gp100 TCR alpha with stabilizing mutations |
| | fused to scFv U1v9 (DtoA)) (TRAC: S21F, T37I, A103T), (Disulfide |
| | stabilized with TRAC: T84C) (PL427_PS1463)) |
| 104 | AIQMTQSPSSLSASVGDRVTITCRASQDIRNYLNWYQQKPGKAPKLLIYYTSRLESGVPSRFSGSGSG |
| | TDYTLTISSLQPEDFATYYCQQGNTLPWTFGQGTKVEIKGGGGSGGGGSGGGGSGGGGSGGGSEVQLV |
| | ESGGGLVQPGGSLRLSCAASGYSFTGYTMNWVRQAPGKGLEWVALINPYKGVSTYNQKFKDRFTISVD |
| | KSKNTAYLQMNSLRAEDTAVYYCARSGYYGDSDWYFDVWGQGTLVTVSSGGGGSGGGGSGGGGSGGGG |
| | SGGGGSQEDERIVLVDNKCKCARITSRIIRSSEDPNEDIVERNIRIIVPLNNRENISDPTSPLRTRFV |
| | YHLSDLCKKCDPTEVELDNQIVTATQSNICDEDSATETCYTYDRNKCYTAVVPLVYGGETKMVETALT |
| | PDACYPD |
| | (Human Ig Jch with anti-CD3 (UCHT1v9(DIA)) at N-terminus) |
| 105 | QEDERIVLVDNKCKCARITSRIIRSSEDPNEDIVERNIRIIVPLNNRENISDPTSPLRTRFVYHLSDL |
| | CKKCDPTEVELDNQIVTATQSNICDEDSATETCYTYDRNKCYTAVVPLVYGGETKMVETALTPDACYP |
| | DGGGGSGGGGSGGGGSGGGGSGGGGSAIQMTQSPSSLSASVGDRVTITCRASQDIRNYLNWYQQKPGK |
| | APKLLIYYTSRLESGVPSRFSGSGSGTDYTLTISSLQPEDFATYYCQQGNTLPWTFGQGTKVEIKGGG |
| | GSGGGGSGGGGSGGGGSGGGSEVQLVESGGGLVQPGGSLRLSCAASGYSFTGYTMNWVRQAPGKGLEW |
| | VALINPYKGVSTYNQKFKDRFTISVDKSKNTAYLQMNSLRAEDTAVYYCARSGYYGDSDWYFDVWGQG |
| | TLVTVSS |
| | (Human Ig J chain with anti-CD3 (UCHT1v9 (DIA)) at C-terminus) |
| 106 | YLEPGPVTV |
| | (heteroclitic gp100 peptide) |

1. A T cell receptor (TCR) multimer comprising a TCR moiety operatively linked to a multimerization moiety, wherein:
(a) the TCR moiety comprises at least one variable (V) region, wherein the V region comprises an α chain V region, a β chain V region, a γ chain V region or a δ chain V region; and
(b) the multimerization moiety comprises (i) an IgM Cμ4 constant region and a tailpiece region; or (ii) an IgA Cα3 constant region and a tailpiece region, wherein the tailpiece region in (b)(i) or (b)(ii) is an IgM tailpiece region or an IgA tailpiece region,
wherein the multimerization moiety comprising the IgM Cμ4 constant region further comprises an IgM Cμ3 constant region, an IgM Cμ2 constant region, an IgM Cμ1 constant region, or any combination thereof,
and wherein the multimerization moiety comprising the IgA Cα3 constant region further comprises an IgA Cα2 constant region, an IgA Cα1 constant region, or any combination thereof,
and wherein the TCR moiety is operatively linked to the multimerization moiety through the TCR α chain, the TCR γ chain, TCR β chain or the TCR δ chain by a linker, wherein the linker is a flexible linker, a rigid linker or a semi-rigid linker.

2-20. (canceled)

21. The TCR multimer of claim 1, which further comprises an immunoglobulin J chain.

22. The TCR multimer of claim 1, which is soluble.

23. The TCR multimer of claim 1, wherein the TCR moiety is a single chain TCR, a dimer, a trimer, a tetramer, or a pentamer.

24-28. (canceled)

29. The TCR multimer of claim 1, wherein the TCR moiety is operatively linked to the multimerization moiety through the TCR α chain, the TCR γ chain, TCR β chain or the TCR δ chain.

30-32. (canceled)

33. The TCR multimer of claim 21, which further comprises an effector moiety operatively linked to the immunoglobulin J chain or to the multimerization domain.

34. (canceled)

35. The TCR multimer of claim 33, wherein the effector moiety comprises a checkpoint protein agonist, an immune cell engaging agent, or a cell-death inducing agent.

36. (canceled)

37. The TCR multimer of claim 35, wherein the effector moiety comprises an immune cell engaging agent, wherein the immune cell engaging agent comprises an anti-CD3 antibody, an anti-NKp46 antibody, an anti-CD16a antibody, an anti-CD56 antibody, an anti-NKG2D antibody, an anti-NKp30 antibody, an anti-CD64 antibody, or any portion or antigen-binding fragments thereof.

38-44. (canceled)

45. The TCR multimer of claim 1, which further comprises one or more heterologous functional domains.

46. A composition comprising a mixture, wherein the mixture comprises (i) at least two multimers of claim 1; and (ii) wherein the at least two multimers are selected from the group consisting of dimers, trimers, tetramers, pentamers, and hexamers.

47. One or more isolated nucleic acid molecules encoding the TCR multimer of claim 1.

48. An expression vector comprising the one or more isolated nucleic acid molecules of claim 47.

49-74. (canceled)

75. A host cell comprising the one or more nucleic acid molecules of claim 47.

76-88. (canceled)

89. A method of preparing a T cell receptor (TCR) multimer comprising culturing the host cell of claim 75, further comprising isolating the TCR multimer from the host cells or the host cell culture supernatant.

90. (canceled)

91. A pharmaceutical composition comprising the TCR multimer of claim 1 and a pharmaceutically acceptable carrier.

92. A kit comprising the TCR multimer of claim 1 packaged in a container with instructions for use in detecting peptide-MHC and/or modulating an immune response.

93. A method of detecting a peptide-MHC complex, the method comprising contacting a peptide-MHC complex with the TCR multimer of claim 1 and detecting binding of the TCR multimer to the peptide-MHC complex to thereby detect the peptide-MHC complex, wherein the peptide-MHC complex is on the surface of an antigen presenting cell (APC) and the TCR multimer is contacted with the APC.

94. (canceled)

95. A method of modulating an immune response in a subject, the method comprising administering to the subject the TCR multimer of claim 1 such that an immune response is modulated in the subject.

96-104. (canceled)

105. A method of treating a cancer, an infectious disease, or an autoimmune disease using an immunotherapy, the method comprising: administering to a subject in need thereof an effective amount of the TCR multimer of claim 1.

106-112. (canceled)

113. The TCR multimer of claim 1, comprising:
(a) a TCR moiety comprising TCR variable and constant regions, IgM Cμ3 and an Cμ4 constant region, and a IgM tailpiece region in the multimerization moiety, and an immunoglobulin J chain;
(b) a TCR moiety comprising TCR variable and constant regions, IgM Cμ2, Cμ3 and Cμ4 constant regions, and a IgM tailpiece region in the multimerization moiety, and an immunoglobulin J chain;
(c) a TCR moiety comprising TCR variable and constant regions, IgA Cα2 and Cα3 constant regions and IgA tailpiece region in the multimerization moiety, and an immunoglobulin J chain;
(d) a TCR moiety comprising TCR variable and constant regions, IgM Cμ3 and an Cμ4 constant region, and a IgM tailpiece region in the multimerization moiety, and an immunoglobulin J chain linked to an effector moiety;
(e) a TCR moiety comprising TCR variable and constant regions, IgM Cμ2, Cμ3 and Cμ4 constant regions, and a IgM tailpiece region in the multimerization moiety, and an immunoglobulin J chain linked to an effector moiety; or
(f) a TCR moiety comprising TCR variable and constant regions, IgA Cα2 and Cα3 constant regions and IgA tailpiece region in the multimerization moiety, and an immunoglobulin J chain linked to an effector moiety.
